(19) United States
(12) Patent Application Publication
(10) Pub. No.: US 2021/0052660 A1
(43) Pub. Date: Feb. 25, 2021

(54) NEURAL STEM CELL COMPOSITIONS INCLUDING CHIMERIC POXVIRUSES FOR CANCER TREATMENT

(71) Applicant: City of Hope, Duarte, CA (US)

(72) Inventors: Alexandra J. ANNALA, Duarte, CA (US); Karen S. ABOODY, Duarte, CA (US); Yuman FONG, Duarte, CA (US); Nanhai G. CHEN, Duarte, CA (US); Mohamed HAMMAD, Duarte, CA (US); Rachael MOONEY, Duarte, CA (US); Jennifer COVELLO, Duarte, CA (US)

(21) Appl. No.: 17/051,353

(22) PCT Filed: Apr. 30, 2019
(86) PCT No.: PCT/US19/30067

§ 371 Date: Oct. 28, 2020

Related U.S. Application Data
(60) Provisional application No. 62/664,269, filed on Apr. 30, 2018.
(60) Provisional application No. 62/672,511, filed on May 16, 2018.

(51) Int. Cl. A61K 35/30; A61P 35/00; A61K 35/768; A61K 9/00; C12N 15/11

(52) U.S. Cl. CPC A61K 35/30; A61P 35/00; C12N 15/11; A61K 9/0019; A61K 35/768

(57) ABSTRACT

Provided are methods and compositions for treating cancer with a combination of neural stem cells (NSCs) and a replication-competent oncolytic virus such as conditionally replication-competent chimeric orthopoxvirus (CF33). The cancer includes but is not limited to primary, recurrent, and metastatic brain cancer, breast cancer, head and neck cancer, bladder cancer, ovarian cancer, uterine cancer, prostate cancer, skin cancer, lung cancer, and colorectal cancer.

### CROSS-REFERENCES TO RELATED APPLICATIONS

This application claims the benefit of U.S. Provisional Application No. 62/664,269, filed Apr. 30, 2018, and U.S. Provisional Application No. 62/672,511, filed May 16, 2018 which are incorporated herein by reference in entirety and for all purposes

### STATEMENT AS TO RIGHTS TO INVENTIONS MADE UNDER FEDERALLY SPONSORED RESEARCH AND DEVELOPMENT

This invention was made with government support under grant no. 1R01CA197359-01 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048440-701001WO-SEQUENCE_LISTING_ST25, created on Apr. 30, 2019, 688,211 bytes, machine format IBM-PC, MS Windows operating system, is hereby incorporated by reference.

### BACKGROUND

Ovarian cancer is the most lethal gynecologic malignancy, afflicting approximately 22,000 women per year in the U.S. (Jemal et al., 2008; and Li et al., 2012). Once ovarian cancer has metastasized to the abdominal cavity (stage III), patients have only a 34% 5-year survival rate following standard treatment with surgical debulking and combination chemotherapy (e.g., cisplatin and paclitaxel) (Cannistra et al., 2004). Use of intraperitoneally (IP) delivered combination chemotherapy regimens has improved outcomes (Kim et al., 2015); however, these regimens frequently have complications and serious toxic side effects such that most patients are unable to complete the treatment cycles due to severe abdominal pain, nausea, and vomiting (Ding 2014). Furthermore, regardless of treatment regimen, most ovarian cancer patients eventually develop chemo-resistance, leading to cancer progression and death. Therefore, new, more targeted and effective therapeutic approaches for treating recurrent and/or drug-resistant ovarian cancer are needed. The methods and compositions provided herein address these and other needs in the art.

### BRIEF SUMMARY OF THE INVENTION

In one aspect, provided herein is a method of treating cancer with neural stem cells mediated delivery of a replication-competent oncolytic virus such as conditionally replication-competent chimeric orthopoxvirus CF33. The method entails administrating to a subject a therapeutically effective amount of neural stem cells and a replication-competent oncolytic virus. In some embodiments, the neural stem cells and the replication-competent oncolytic virus are administered simultaneously. In some embodiments, the neural stem cells and the replication-competent oncolytic virus are administered sequentially. In some embodiments, the neural stem cells and the oncolytic adenovirus are administered by injection, e.g., intraperitoneal injection. In some embodiments, the method further includes administering a therapeutically effective amount of anti-PD-L1, CTLA4 or OX40 to the subject, before, during, or after administration of the neural stem cells and the replication-competent oncolytic virus. In some embodiments, the neural stem cell is from a neural stem cell line HB1.F3.CD. In some embodiments, the neural stem cell is clonal human neural stem cell line HB1.F3.CD21. In some embodiments, the cancer is an intraperitoneal cancer including but not limited to, peritoneal cancer, ovarian cancer, bladder cancer, pancreatic cancer, colorectal cancer, gastric cancer, and liver cancer. In some embodiments, the cancer is metastatic ovarian cancer.

In a related aspect, disclosed herein is a pharmaceutical composition comprising a therapeutically effective amount of neural stem cells and a replication-competent oncolytic virus. In some embodiments, the virus is conditionally replication-competent chimeric orthopoxvirus CF33. In some embodiments, the neural stem cell is from a neural stem cell line HB1.F3.CD. In some embodiments, the neural stem cell is clonal human neural stem cell line HB1.F3.CD21. In some embodiments, the pharmaceutical composition further including a therapeutically effective amount of anti-PD-L1, CTLA4 or OX40. In some embodiments, the pharmaceutical composition further including one or more pharmaceutically acceptable carriers or excipients.

In another aspect is provided a method of treating cancer. The method includes administering to a subject in need thereof an effective amount of a chimeric poxvirus and a neural stem cell (NSC).

In another aspect is provided a composition including a chimeric poxvirus and a neural stem cell (NSC).

In another aspect is provided a neural stem cell (NSC) including a chimeric poxvirus.

In another aspect is provided a method of treating cancer. The method includes administering to a subject in need thereof an effective amount of a neural stem cell as described herein, including embodiments thereof.

### BRIEF DESCRIPTION OF THE DRAWINGS

FIG. 1A-1B. The figures show generation and evaluation of a novel chimeric poxvirus. Construction of CF33, validation of its cancer selectivity, and optimization of CF33 loading dose in NSCs. FIG. 1A: nu/nu mice bearing human OVCAR8 tumors were treated with chimeric orthopoxvirus CF33. Tumor volume was measured at days 0, 7, 14, 26, and 34 after treatment. FIG. 1B: Cell viability over time for MOIs ranging from 0.001 to 10 of CH33.

FIG. 2A-2I. The figures show IP administered NSCs are selectively tropic to peritoneal ovarian cancer metastases. Ex vivo images of (FIG. 2A) human OVCAR8.ffluc nu/nu immunodeficient model. (FIG. 2B) mouse ID8.tdTomato syngeneic C57Bl/6 immunocompetent model. Insets for FIG. 2A and FIG. 2B show harvested tumors ex vivo with bioluminescent imaging for ffluc. (FIG. 2C) Note predominance of metastases to omentum, diaphragm and peritoneal surface (FIGS. 2D-2H) Representative fluorescent images from harvested peritoneal tumors after NSC administration. (FIGS. 2D-2F) NSCs labeled with CellTracker CM-DiI (red) demonstrate distribution in tumor but not in adjacent normal kidney tissue (2×10⁶ NSC.DiI in 200 μL of PBS injected IP on day 38 and harvested 4 days post-NSC injection) (tumor labeled with eGFP, green), DAPI (nuclei blue). (FIGS. 2G-2H) NSCs with surface-conjugated nanoparticles (orange) demonstrate good distribution in tumor but not adjacent normal liver tissue (4×10⁶ NSC.NP in 1 mL of PBS injected IP on Day 13 and harvested 4 days post-NSC injection. Scale bar for panels FIG. 2D, 2G: 1000 μm; FIG. 2E, 2H: 500 μm, FIG. 2F, 2I: 200 μm.

FIG. 3A-3B. The figures show CF33 infects and kills ID8 mouse ovarian cancer cell line in vitro. Ovarian cancer cell lines are populated by CF33 NSCs in vitro. (FIG. 3A) CF33-eGFP NSCs co-cultured with ID8 cells (1:1000) infected tumor cells, as indicated by increased intensity of GFP until cells died. Intensity then decreased. NSCs cell not infected with CF33 do not exhibit any green fluorescence signal. (FIG. 3B) Graphical analysis of confluency mask for the cells in panel A showing that CF33 NSCs kill ID8 cell when co-cultured with them at a low NSC:tumor cell ratio (1:1000).

FIG. 4A-4B. The figures show CF33-NSC delivery of virus selectively to ovarian metastases. NSC-delivered chimeric orthopoxvirus CF33 preferentially infects IP ovarian tumors. OVCAR8 tumor-bearing nu/nu mice (n=5; 21 days post tumor implantation) were treated with IP CF33-NSCs (2×10⁶/200 μl/mouse; 5 i.u./cell). Tumors with adjacent tissue were harvested 2 days later. (FIG. 4A) Representative micrograph of a tumor mass (black dotted line) stained with ffluc antibody-DAB (brown). (FIG. 4B) Sister serial section of the section shown in A stained with an anti-pox antibody-DAB (brown). This staining pattern suggests that CF33 delivered by NSCs spreads through tumors from the periphery inwards (black arrows). Scale bars: 200 μm

FIG. 5A-5D. The figure shows NSCs improved distribution of CF33 in vivo. NSCs selectively seed CF33 in ovarian tumors with improved distribution over free CF33 in immunocompetent model. C57Bl/6 mice with established IP ID8 tumors with CF33-NSCs (2×10⁶) or free CF33 with matched viral load 3×10⁶ plaque forming units (PFU), or PBS control. Immunofluorescence staining with antivaccinia Ab (red) confirmed transfer of CF33 to tumor (green) in the CF33-NSC group (FIG. 5A) and free CF33 group (FIG. 5B) with matched viral loads (3×10⁶ pfu/mouse), compared with the untreated group (FIG. 5C), 4 days after 1st treatment. Scale bar=100 μm, (FIG. 5D) NSCs localize to IP ovarian tumor foci within 1 hour after administration. % AuNR was quantified by ICP mass spectroscopy. No significant difference was detected between 1 and 72 hours post AuNRNSC administration.

FIG. 6. The figure shows NSCs shielded CF33 from immune neutralization. NSCs appear to shield CF33 from immune neutralization. In vivo BLI of C57Bl/6 mice with established ID8 peritoneal metastases 1 hour after treatment with free CF33.ffluc (left) or CF33.ffluc-NSCs (right). Bottom panels show BLI 1 hour after a 2nd treatment round given 1 week later. Graph shows quantification of CF33.ffluc BLI signal. Note CF33 delivered by NSCs shows significant increase in viral load at tumor sites 1 h after both 1st (p=0.0193) and 2^{nd}.

FIG. 7. The figure shows NSCs improved CF33 distribution even for 24 hours after 2^{nd} treatment round. NSC-mediated viral distribution to IP ovarian metastases is enhanced 1 day after 2nd treatment. C57BL/6 mice received IP ID8 ovarian cancer cells (5×10⁶/200 μl/mouse). Treatments were given at 3 weeks and 6 weeks after tumor establishment, with matched viral load for CF33-NSCs and CF33 (1×10⁷ CF33 PFU in 2×10⁶ NSCs). Virus retention at individual ovarian tumor foci 1 day post treatment was greater after a 2^{nd} round of treatment.

FIG. 8. The figure shows free novel chimeric orthpoxviruses show superior tumoricidal activity in NCI-60 cell lines.

FIG. 9. The figure shows free novel chimeric orthopoxvirus shrinks triple-negative breast cancer. The viral dose used was 2-5 orders of magnitude lower than the oncolytic viruses under clinical testing.

FIG. 10. The figure shows free novel chimeric poxvirus (HOV) inhibits the growth of distant, untreated tumors from NCI-60 cancer cell lines.

FIG. 11. The figure shows free novel chimeric poxvirus (HOV) infects fresh human triple-negative breast cancer ex vivo. GFP version of the novel chimeric poxvirus.

FIG. 12. The figure shows in vitro cytotoxicity and viral replication of free chimeric orthopoxvirus CF33-Fluc in pancreatic cancer cell lines.

FIG. 13. The figure shows free CF33 causes regression of both injected and non-injected distant tumors at a low dose in pancreatic xenograft models.

FIG. 14A-14C. The figure shows virus-encoded luciferase activity correlates with tumor regression over time. Virus is distributed to distant non-injected tumors as measured by virus-encoded luciferase activity (FIG. 14A). Decrease in tumor size correlates with virus-encoded luciferase activity (FIG. 14B). Increasing viral titers are observed in non-injected tumors, showing good distribution of CF33-Fluc (FIG. 14C).

FIG. 15. The figure shows histology and immunochemistry of tumor sections confirm killing of free CF33-Fluc injected and non-injected, distant tumors.

FIG. 16. The figure shows free CF33 shrinks ovarian cancer as demonstrated by decrease in tumor volume.

FIG. 17. The figure shows a schematic map of available viruses used for the methods and compositions provided herein.

FIG. 18. The figure shows in vitro comparative tumor killing study. Compared CF33 EGFP (black circle), CF33 Hs-Luc2 (gray asterisk), CF33 11K-Luc2 (black square), CF17 (black triangle), CF189 (gray triangle), and a control (gray diamond) for tumor killing of ID8 (mouse ovarian cancer) and ovcar8 (human ovarian cancer) at various MOIs (1:10, 1:100 and 1:1000).

FIG. 19A-19B. The figure shows in vitro NSC-CF33.11k-Luc Viability Post Incubation (FIG. 19A). NSC-CF33.11K-Luc Viability Post Thaw (FIG. 19B).

FIG. 20. The figure shows the experimental design to determine the efficacy of checkpoint inhibitors anti-PD-1, anti-CTLA-4, or a combination thereof. Tumor inoculation (ID8 ovarian CBR LUC) occurred at day 0, with 4×10⁶ in 100 μl PBS/IP injected intraperitoneally (i.p.). The checkpoint inhibitors are administered at indicated times (day 0 (DO), day 4 (D4), day 5 (D5), day 7 (D7), day 9 (D9) or day 14 (D14), respectively) after tumor inoculation and treatment with free CF33 or NSC-CF33. On day 4, either CF33/11K-Luc2 NSCs 2×10⁶ or free CF33/11k-Luc2 were injected i.p. On day 5, either PD-1 Ab, CTLA-4 Ab or a combination thereof was administered. On day 7, either PD-1 Ab or a combination of PD-1Ab and CTLA-4 Ab was administered. On day 9, either PD-1 Ab or a combination of PD-1Ab and CTLA-4 Ab was administered. On day 14, either CF33/11K-Luc2 NSCs 2×10⁶ or free CF33/11k-Luc2 were injected i.p.

FIG. 21. The figure shows CF33 mCherry NSCs remarkably seeded more CF33 in a glioblastoma syngeneic immunocompetent mouse model after 1 round of IV injection compared with free CF33 mCherry. Brains were harvested 2 days after IV injection, n=2 mice/group.

FIG. 22. The figure shows CF33 upregulates PD-L1 expression in ID8 tumor cells in vitro. Flow Cytometry for PD-L1 expression in ID8 tumor cells. ID8 cells in vitro were treated with 0.5 M cisplatin and CF33-NSCs at a ratio of 1:1000 and 1:100. Treated cells were harvested after 2 days of culture and stained with a PE PD-L1 monoclonal antibody for FACS analysis. Significant upregulation of PD-L1 was noted in ID8 cells exposed to CF33-NSCs at MOIs of 0.001 and 0.01. PD-L1 monoclonal antibody clone 10F.9G2 was purchased from BioLegend (San Diego, Calif.) Cells were analyzed by a Guava easyCyte HT flow cytometer using InCyte and FCS Express 6 software.

FIG. 23. The figure shows OV-VAC 17-04 Tumor Challenge Schema. Tumor Challenge includes implanting ID8 ovarian LV TDTOM 1.0×10⁶ by i.p. injection of 14 week old female C57BL6 mice on day 0 (DO). Treatment rounds 1, 2 and 3 are administered at indicated times (day 28 (D28), day 35 (D35), and day 42 (D42), respectively) after tumor inoculation. Treatment rounds include i.p. injection of 500 uL of either 3.0×10⁶ pfu virus or 2.0×10⁶ NSCs-Virus in 2 groups of 4 mice per group. Whole body bioluminescent imaging (BLI) is completed at indicated intervals following treatment, days 28, 29, 30, 31, and 32 (D28, 29, 30, 31, and 32), days 35, 36, 37, 38, and 39 (D35, 36, 37, 38, and 39), and days 42, 43, 44, 45, and 46 (D42, 43, 44, 45, and 46). Mice are euthanized on day 65 (D65). Harvesting is conducted post-euthanization by collecting the pluck, diaphragm and peritoneal fluid.

FIG. 24. The figure shows mouse plasma carbohydrate antigen 125 (CA125) levels pre and post tumor implantation, and post treatment with either free FFluc-CF33 or FFluc-CF33 carried by NSCs.

FIG. 25. The figure shows combination of CF33-NSC and PD-1 blockade remarkably prolonged survival in a syngeneic mouse model of ovarian cancer.

FIG. 26. The figure shows combined oncolytic virus and checkpoint blockade therapy is highly effective against cancer.

FIG. 27. The figure shows bioluminescent measurements post tumor implantation following treatment with free CF33 virus, CF33-NSC, immune checkpoint inhibitor PD-1 Ab, and sequential treatment of CF33-NSC and PD-1 Ab.

### DETAILED DESCRIPTION

Methods for treating cancer using tropic cells (e.g., stem cells or neural stem cells (NSCs)) that carry a replication-competent oncolytic virus are provided herein. In some embodiments, the virus is conditionally replication-competent chimeric orthopoxvirus CF33. In some embodiments, the virus is conditionally replication-competent chimeric orthopoxvirus CF17. In some embodiments, the NSCs are HB1.F3.CD21. Such methods may be used to treat an intraperitoneal cancer capable of being treated via intraperitoneal (IP) injection including but not limited to, peritoneal cancer, ovarian cancer, bladder cancer, pancreatic cancer, colorectal cancer, gastric cancer, and liver cancer. In some embodiments, the cancer is metastatic ovarian cancer.

Replication-competent oncolytic virotherapy offers a new, highly promising approach for treating ovarian cancer. Once seeded into the tumor, the oncolytic virus can selectively replicate in tumor cells (but not in normal tissue) to destroy tumor cells in situ via direct lysis. Importantly, oncolytic viruses can induce cancer cell death irrespective of radio- or chemoresistance and can also stimulate immune system recognition of cancer cells by exposing tumor antigens upon lysis. Clinical efficacy of this approach has been limited by rapid viral inactivation by the immune system, poor viral penetration of tumors, and an inability of the virus to effectively reach invasive metastatic foci separated by normal tissue.

Disclosed herein is a novel conditionally replication-competent chimeric orthopoxviruses (CF33 and CF17) selected specifically for ovarian cancer cell infection, following screening of over 100 chimeric poxviruses. The CF33 virus was engineered to be tumor specific by deleting the virus's thymidine kinase (TK) gene. Tumor tissues have abnormally high TK expression that complements this gene deletion, permitting viral replication, whereas normal tissue does not. The chimeric poxviruses viruses (e.g., CF33 and CF17) described in PCT Application Publication No. WO 2018/031694, entitled “Chimeric Poxvirus Compositions and Uses Thereof,” which is hereby incorporated by reference in its entirety and for all purposes, may be used for the methods and compositions provided herein. Some examples of the CF33 viruses used herein include CF33/SE-HNIS ANTI-PDL1, CF33/H5-FFLUC, CF33/H5-MCHERRY, and CF33/H5-EMERALD GFP.

As disclosed herein a modified tumor-tropic, HLA II-negative neural stem cell line (HB1.F3.CD21 NSCs; demonstrating clinical safety in high-grade glioma patients)^{[20,95]} efficiently delivers CF33 to ovarian cancer foci in preclinical models. NSCs provide protection from immune-mediated clearance and neutralization to achieve effective viral distribution to peritoneal ovarian tumors. Co-culture in vitro experiments of NSC-delivered CF33 in human OVCAR8 and murine ID8 ovarian cancer cells showed robust infection of >95% of tumor cells in 6 days, even at a low ratio of 1 NSC:1000 tumor cells. In vivo data show selective distribution of NSC-delivered CF33 to human OVCAR8 and ID8 peritoneal metastases in immunodeficient and immunocompetent mice, respectively. Furthermore, IP-delivered CF33-NSCs showed increased viral distribution to tumor sites (assessed by BLI, IHC, and qPCR) compared to a matched viral load of free CF33 when multiple treatment rounds were given in the immunocompetent model. Long-term efficacy studies can establish the clearance kinetics, immune response, viral distribution, and efficacy of NSC-delivered vs. free CF33. CF33-NSCs can be used for selective tumor killing in patients suffering from various cancers, including stage III ovarian cancer.

### I. Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

An amino acid residue in a protein “corresponds” to a given residue when it occupies the same essential structural position within the protein as the given residue.

The terms “isolate” or “isolated”, when applied to a nucleic acid, virus, or protein, denotes that the nucleic acid, virus, or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

The term “peptidyl” and “peptidyl moiety” means a monovalent peptide.

A “ligand” refers to an agent, e.g., a polypeptide or other molecule, capable of binding to a receptor.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine.; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A “labeled nucleic acid or oligonucleotide” is one that is bound, either covalently, through a linker or a chemical bond, or noncovalently, through ionic, van der Waals, electrostatic, or hydrogen bonds to a label such that the presence of the nucleic acid may be detected by detecting the presence of the detectable label bound to the nucleic acid. Alternatively, a method using high affinity interactions may achieve the same results where one of a pair of binding partners binds to the other, e.g., biotin, streptavidin. In embodiments, the phosphorothioate nucleic acid or phosphorothioate polymer backbone includes a detectable label, as disclosed herein and generally known in the art.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

The term “bispecific T-cell engager (BiTE)”, “BiTe” or “bispecific antibody” as provided herein is used according to its conventional meaning well known in the art and refers to a bispecific recombinant protein capable to simultaneously bind to two different antigens. In contrast to traditional monoclonal antibodies, BiTE antibodies consist of two independently different antibody regions (e.g., two single-chain variable fragments (scFv)), each of which binds a different antigen. One antibody region engages effector cells (e.g., T cells) by binding an effector cell-specific antigen (e.g., CD3 molecule) and the second antibody region binds a target cell (e.g., cancer cell or autoimmune-reactive cell) through a cell surface antigen (e.g., BAFF-R) expressed by said target cell. Binding of the BiTE to the two antigens will link the effector cell (e.g., T cell) to the target cell (e.g., tumor cell) and activate the effector cell (e.g., T cell) via effector cell-specific antigen signaling (e.g., CD3 signaling). The activated effector cell (e.g., T cell) will then exert cytotoxic activity against the target cell (e.g., tumor cells).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

An “antisense nucleic acid” as referred to herein is a nucleic acid (e.g., DNA or RNA molecule) that is complementary to at least a portion of a specific target nucleic acid and is capable of reducing transcription of the target nucleic acid (e.g. mRNA from DNA), reducing the translation of the target nucleic acid (e.g. mRNA), altering transcript splicing (e.g. single stranded morpholino oligo), or interfering with the endogenous activity of the target nucleic acid. See, e.g., Weintraub, Scientific American, 262:40 (1990). Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid. In embodiments, the antisense nucleic acid hybridizes to the target nucleic acid in vitro. In embodiments, the antisense nucleic acid hybridizes to the target nucleic acid in a cell. In embodiments, the antisense nucleic acid hybridizes to the target nucleic acid in an organism. In embodiments, the antisense nucleic acid hybridizes to the target nucleic acid under physiological conditions. Antisense nucleic acids may comprise naturally occurring nucleotides or modified nucleotides such as, e.g., phosphorothioate, methylphosphonate, and -anomeric sugar-phosphate, backbonemodified nucleotides.

In the cell, the antisense nucleic acids hybridize to the corresponding RNA forming a double-stranded molecule. The antisense nucleic acids interfere with the endogenous behavior of the RNA and inhibit its function relative to the absence of the antisense nucleic acid. Furthermore, the double-stranded molecule may be degraded via the RNAi pathway. The use of antisense methods to inhibit the in vitro translation of genes is well known in the art (Marcus-Sakura, Anal. Biochem., 172:289, (1988)). Further, antisense molecules which bind directly to the DNA may be used. Antisense nucleic acids may be single or double stranded nucleic acids. Non-limiting examples of antisense nucleic acids include siRNAs (including their derivatives or pre-cursors, such as nucleotide analogs), short hairpin RNAs (shRNA), micro RNAs (miRNA), saRNAs (small activating RNAs) and small nucleolar RNAs (snoRNA) or certain of their derivatives or pre-cursors.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

A “siRNA,” “small interfering RNA,” “small RNA,” or “RNAi” as provided herein refers to a nucleic acid that forms a double stranded RNA, which double stranded RNA has the ability to reduce or inhibit expression of a gene or target gene when expressed in the same cell as the gene or target gene. The complementary portions of the nucleic acid that hybridize to form the double stranded molecule typically have substantial or complete identity. In one embodiment, a siRNA or RNAi refers to a nucleic acid that has substantial or complete identity to a target gene and forms a double stranded siRNA. In embodiments, the siRNA inhibits gene expression by interacting with a complementary cellular mRNA thereby interfering with the expression of the complementary mRNA. Typically, the nucleic acid is at least about 15-50 nucleotides in length (e.g., each complementary sequence of the double stranded siRNA is 15-50 nucleotides in length, and the double stranded siRNA is about 15-50 base pairs in length). In other embodiments, the length is 20-30 base nucleotides, preferably about 20-25 or about 24-29 nucleotides in length, e.g., 20, 21, 22, 23, 24, 25, 26, 27, 28, 29, or 30 nucleotides in length. Non-limiting examples of siRNAs include ribozymes, RNA decoys, short hairpin RNAs (shRNA), micro RNAs (miRNA) and small nucleolar RNAs (snoRNA).

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “heterologous” when used with reference to portions of a nucleic acid indicates that the nucleic acid including two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein including two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “VII,” or “VH” refer to the variable region of an immunoglobulin heavy chain, including an Fv, scFv, dsFv or Fab; while the terms “variable light chain,” “VL” or “VL” refer to the variable region of an immunoglobulin light chain, including of an Fv, scFv, dsFv or Fab.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

Antibodies exist, for example, as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially the antigen binding portion with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of an antibody is the region of its antigen to which the antibody binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

The term “antigen” as provided herein refers to molecules capable of binding to the antibody binding domain provided herein. An “antigen binding domain” as provided herein is a region of an antibody that binds to an antigen (epitope). As described above, the antigen binding domain is generally composed of one constant and one variable domain of each of the heavy and the light chain (VL, VH, CL and CH1, respectively). The paratope or antigen-binding site is formed on the N-terminus of the antigen binding domain. The two variable domains of an antigen binding domain typically bind the epitope on an antigen.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

A “CTLA-4” or “CTLA-4 protein” as referred to herein includes any of the recombinant or naturally-occurring forms of cytotoxic T-lymphocyte-associated protein 4 (CTLA-4) or variants or homologs thereof that maintain CTLA-4 activity (e.g. within at least 50%, 80%, 90%, 95%, 96%, 97%, 98%, 99% or 100% activity compared to CTLA-4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CTLA-4 polypeptide. In embodiments, the CTLA-4 protein is substantially identical to the protein identified by the NCBI reference number GI: 83700231, or a variant or homolog having substantial identity thereto. In embodiments, the CTLA-4 protein is substantially identical to the protein identified by the NCBI reference number GI: 49902519, or a variant or homolog having substantial identity thereto. In embodiments, the CTLA-4 protein is substantially identical to the protein identified by the NCBI reference number GI: 49904741, or a variant or homolog having substantial identity thereto. In embodiments, the CTLA-4 protein is substantially identical to the protein identified by the NCBI reference number GI: 291929, or a variant or homolog having substantial identity thereto.

The term “CTLA-4 inhibitor” as provided herein refers to a substance (e.g., compound, protein, peptide, small molecule, antibody) capable of detectably lowering expression or activity level of a cytotoxic T-lymphocyte-associated protein 4 (CTLA-4) compared to a control. The inhibited expression or activity of the cytotoxic T-lymphocyte-associated protein 4 (CTLA-4) can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control. An “antagonist” is a compound or small molecule that inhibits an cytotoxic T-lymphocyte-associated protein 4 (CTLA-4) e.g., by binding, partially or totally blocking stimulation, decrease, prevent, or delay activation, or inactivate, desensitize, or down-regulate signal transduction, gene expression or enzymatic activity necessary for CTLA-4 activity. In embodiments, the CTLA-4 antagonist is a compound or a small molecule. In embodiments, the CTLA-4 antagonist is an antibody.

The terms “D4R gene”, “uracil DNA glycosylase gene”, or the like, as used herein refer to the any of the recombinant or naturally-occurring forms of the uracil DNA glycosylase gene or variants or homologs thereof that code for a uracil DNA glycosylase polypeptide capable of maintaining the activity of the uracil DNA glycosylase polypeptide (e.g., within at least 50%, 80%, 90%, 95%, 96%, 97%, 98%, 99% or 100% activity compared to uracil DNA glycosylase polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring uracil DNA glycosylase gene. In embodiments, the uracil DNA glycosylase gene is substantially identical to the nucleic acid sequence corresponding to position 102720-103376 of the nucleic acid sequence identified by Accession No. DQ439815 or a variant or homolog having substantial identity thereto. In embodiments, the uracil DNA glycosylase gene includes the nucleic acid sequence of SEQ ID NO:8. In embodiments, the uracil DNA glycosylase gene is the nucleic acid sequence of SEQ ID NO:8.

The terms “E9L gene”, “DNA polymerase gene”, or the like, as used herein refer to the any of the recombinant or naturally-occurring forms of the DNA polymerase gene or variants or homologs thereof that code for a DNA polymerase polypeptide capable of maintaining the activity of the DNA polymerase polypeptide (e.g., within at least 50%, 80%, 90%, 95%, 96%, 97%, 98%, 99% or 100% activity compared to DNA polymerase polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring DNA polymerase gene. In embodiments, the DNA polymerase gene is substantially identical to the nucleic acid sequence corresponding to position 56656-53636 of the nucleic acid sequence identified by Accession No. AY243312 or a variant or homolog having substantial identity thereto. In embodiments, the DNA polymerase gene includes the nucleic acid sequence of SEQ ID NO:12. In embodiments, the DNA polymerase gene is the nucleic acid sequence of SEQ ID NO:12.

The terms “human sodium and iodide symporter gene”, “hNIS gene”, “NIS gene” or the like, as used herein refer to the any of the recombinant or naturally-occurring forms of the human sodium and iodide symporter gene or variants or homologs thereof that code for a human sodium and iodide symporter polypeptide capable of maintaining the activity of the human sodium and iodide symporter polypeptide (e.g., within at least 50%, 80%, 90%, 95%, 96%, 97%, 98%, 99% or 100% activity compared to human sodium and iodide symporter polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring human sodium and iodide symporter gene. In embodiments, the human sodium and iodide symporter gene is substantially identical to the nucleic acid sequence identified by Accession No. NM_000453 or a variant or homolog having substantial identity thereto. In embodiments, the human sodium and iodide symporter gene includes the nucleic acid sequence of SEQ ID NO:13. In embodiments, the human sodium and iodide symporter gene is the nucleic acid sequence of SEQ ID NO:13.

The terms “Emerald gene” or “Emerald sequence” as used herein refer to the genetically engineered gene or variants thereof that code for an Emerald polypeptide capable of maintaining the activity of the Emerald polypeptide (e.g., within at least 50%, 80%, 90%, 95%, 96%, 97%, 98%, 99% or 100% activity compared to the Emerald polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to the Emerald sequence. In embodiments, Emerald is substantially identical to the nucleic acid sequence corresponding to position 3215-3931 of the nucleic acid sequence identified by Accession No. KF293661 or a variant or homolog having substantial identity thereto. In embodiments, the Emerald gene includes the nucleic acid sequence of SEQ ID NO:14. In embodiments, the Emerald gene is the nucleic acid sequence of SEQ ID NO:14.

The term “EGFR protein” or “EGFR” as used herein includes any of the recombinant or naturally-occurring forms of epidermal growth factor receptor (EGFR) also known as ErbB-1 or HER1 in humans, or variants or homologs thereof that maintain EGFR activity (e.g. within at least 50%, 80%, 90%, 95%, 96%, 97%, 98%, 99% or 100% activity compared to EGFR). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR protein. In embodiments, the EGFR protein is substantially identical to the protein identified by the UniProt reference number P00533 or a variant or homolog having substantial identity thereto.

The terms “firefly luciferase gene” or “firefly luciferase sequence”, as used herein refer to the any of the recombinant or naturally-occurring forms of the firefly luciferase gene or variants or homologs thereof that code for a firefly luciferase polypeptide capable of maintaining the activity of the firefly luciferase polypeptide (e.g., within at least 50%, 80%, 90%, 95%, 96%, 97%, 98%, 99% or 100% activity compared to firefly luciferase polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring firefly luciferase gene. In embodiments, the firefly luciferase gene is substantially identical to the nucleic acid sequence corresponding to position 3129-4781 of the nucleic acid sequence identified by Accession No. KF990214 or a variant or homolog having substantial identity thereto. In embodiments, the firefly luciferase gene includes the nucleic acid sequence of SEQ ID NO:15. In embodiments, the firefly luciferase gene is the nucleic acid sequence of SEQ ID NO:15.

A “firefly luciferase” or “firefly luciferase protein” as referred to herein includes any of the recombinant or naturally-occurring forms of Firefly luciferase (FFLuc) or variants or homologs thereof that maintain firefly luciferase activity (e.g. within at least 50%, 80%, 90%, 95%, 96%, 97%, 98%, 99% or 100% activity compared to firefly luciferase). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring firefly luciferase protein. In embodiments, the firefly luciferase protein is substantially identical to the protein identified by the NCBI reference number GI: 115342896, or a variant or homolog having substantial identity thereto. In embodiments, the firefly luciferase protein is substantially identical to the protein identified by the NCBI reference number GI: 1176860391, or a variant or homolog having substantial identity thereto. In embodiments, the firefly luciferase protein is substantially identical to the protein identified by the NCBI reference number GI: 7981033, or a variant or homolog having substantial identity thereto. In embodiments, the firefly luciferase protein is substantially identical to the protein identified by the NCBI reference number GI: 115342900, or a variant or homolog having substantial identity thereto.

An “enhanced green fluorescent protein” as referred to herein includes any of the recombinant or naturally-occurring forms of Enhanced green fluorescent protein (eGFP) or variants or homologs thereof that maintain enhanced green fluorescent protein activity (e.g. within at least 50%, 80%, 90%, 95%, 96%, 97%, 98%, 99% or 100% activity compared to enhanced green fluorescent protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring enhanced green fluorescent protein. In embodiments, the enhanced green fluorescent protein is substantially identical to the protein identified by the NCBI reference number GI: 13194618, or a variant or homolog having substantial identity thereto. In embodiments, the enhanced green fluorescent protein is substantially identical to the protein identified by the NCBI reference number GI: 1373316, or a variant or homolog having substantial identity thereto. In embodiments, the enhanced green fluorescent protein is substantially identical to the protein identified by the NCBI reference number GI: 1377909, or a variant or homolog having substantial identity thereto.

A “Hepatitis A virus cellular receptor 2” or “Hepatitis A virus cellular receptor 2 protein” as referred to herein includes any of the recombinant or naturally-occurring forms of Hepatitis A virus cellular receptor 2 (HAVCR2) also known as cluster of T-cell immunoglobulin and mucin-domain containing3 (TIM-3) or variants or homologs thereof that maintain TIM-3 activity (e.g. within at least 50%, 80%, 90%, 95%, 96%, 97%, 98%, 99% or 100% activity compared to TIM-3). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TIM-3 protein. In embodiments, the TIM-3 protein is substantially identical to the protein identified by the NCBI reference number GI: 397787781, or a variant or homolog having substantial identity thereto. In embodiments, the TIM-3 protein is substantially identical to the protein identified by the NCBI reference number GI: 18088589, or a variant or homolog having substantial identity thereto. In embodiments, the TIM-3 protein is substantially identical to the protein identified by the NCBI reference number GI: 49574534, or a variant or homolog having substantial identity thereto.

A “Lymphocyte-activation gene 3” or “Lymphocyte-activation gene 3 protein” as referred to herein includes any of the recombinant or naturally-occurring forms of Lymphocyte-activation gene 3 (LAG-3) also known as cluster of differentiation 223 (CD 223) or variants or homologs thereof that maintain LAG-3 activity (e.g. within at least 50%, 80%, 90%, 95%, 96%, 97%, 98%, 99% or 100% activity compared to LAG-3). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring LAG-3 protein. In embodiments, the LAG-3 protein is substantially identical to the protein identified by the NCBI reference number GI: 649136313, or a variant or homolog having substantial identity thereto. In embodiments, the LAG-3 protein is substantially identical to the protein identified by the NCBI reference number GI: 30851187, or a variant or homolog having substantial identity thereto. In embodiments, the LAG-3 protein is substantially identical to the protein identified by the NCBI reference number GI: 6678654, or a variant or homolog having substantial identity thereto.

A “Indoleamine-pyrrole 2,3-dioxygenase” or “Indoleamine-pyrrole 2,3-dioxygenase protein” as referred to herein includes any of the recombinant or naturally-occurring forms of Indoleamine-pyrrole 2,3-dioxygenase (IDO) also known as INDO or variants or homologs thereof that maintain IDO activity (e.g. within at least 50%, 80%, 90%, 95%, 96%, 97%, 98%, 99% or 100% activity compared to IDO). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IDO protein. In embodiments, the IDO protein is substantially identical to the protein identified by the NCBI reference number GI: 4504577, or a variant or homolog having substantial identity thereto. In embodiments, the IDO protein is substantially identical to the protein identified by the NCBI reference number GI: 1464315781, or a variant or homolog having substantial identity thereto. In embodiments, the IDO protein is substantially identical to the protein identified by the NCBI reference number GI: 333384361, or a variant or homolog having substantial identity thereto.

A “GITR” or “GITR” protein as referred to herein includes any of the recombinant or naturally-occurring forms of the Tumor necrosis factor receptor superfamily member 18 (TNFRSF18) also known as activation-inducible TNFR family receptor (AITR) or glucocorticoid-induced TNFR-related protein (GITR) or variants or homologs thereof that maintain GITR protein activity (e.g. within at least 50%, 80%, 90%, 95%, 96%, 97%, 98%, 99% or 100% activity compared to GITR). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring GITR. In embodiments, the GITR protein is substantially identical to the protein identified by the NCBI reference number GI: 158931986, or a variant or homolog having substantial identity thereto. In embodiments, the GITR protein is substantially identical to the protein identified by the NCBI reference number GI: 4759246, or a variant or homolog having substantial identity thereto. In embodiments, the GITR protein is substantially identical to the protein identified by the NCBI reference number GI: 4378800, or a variant or homolog having substantial identity thereto.

The term “F14.5L gene”, “F14.5L sequence”, “F14.5L”, or the like, as used herein refers to the any of the recombinant or naturally-occurring forms of the F14.5L gene or variants or homologs thereof that code for a F14.5L polypeptide capable of maintaining the activity of the F14.5L polypeptide (e.g., within at least 50%, 80%, 90%, 95%, 96%, 97%, 98%, 99% or 100% activity compared to F14.5L polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring F14.5L gene. In embodiments, the F14.5L gene is substantially identical to the nucleic acid sequence corresponding to position 44428-44279 of the nucleic acid sequence identified by Accession No. KX781953 or a variant or homolog having substantial identity thereto. In embodiments, the F14.5L gene includes the nucleic acid sequence of SEQ ID NO:6. In embodiments, the F14.5L gene is the nucleic acid sequence of SEQ ID NO:6. In embodiments, the F14.5L gene is mutated. In embodiments, the F14.5L gene is partially deleted. In embodiments, the F14.5L gene includes the nucleic acid sequence of SEQ ID NO:7. In embodiments, the F14.5L gene includes the nucleic acid sequence of SEQ ID NO:7.

The terms “mCherry gene” or “mCherry sequence” as used herein refer to the any of the recombinant or naturally-occurring forms of the gene or variants or homologs thereof that code for a mCherry polypeptide capable of maintaining the activity of the mCherry polypeptide (e.g., within at least 50%, 80%, 90%, 95%, 96%, 97%, 98%, 99% or 100% activity compared to mCherry polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring mCherry gene. In embodiments, the mCherry gene is substantially identical to the nucleic acid sequence corresponding to position 1073-1783 of the nucleic acid sequence identified by Accession No. KX446949 or a variant or homolog having substantial identity thereto. In embodiments, the mCherry gene includes the nucleic acid sequence of SEQ ID NO:16. In embodiments, the mCherry gene is the nucleic acid sequence of SEQ ID NO:16.

An “mCherry” or “mCherry protein” as referred to herein includes any of the recombinant or naturally-occurring forms of the mFruits family of monomeric red fluorescent proteins (RFPs) member mCherry, or variants or homologs thereof that maintain mCherry activity (e.g. within at least 50%, 80%, 90%, 95%, 96%, 97%, 98%, 99% or 100% activity compared to mCherry). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring mCherry protein. In embodiments, the mCherry protein is substantially identical to the protein identified by the NCBI reference number GI: 1229406991, or a variant or homolog having substantial identity thereto. In embodiments, the mCherry protein is substantially identical to the protein identified by the NCBI reference number GI: 1041520567, or a variant or homolog having substantial identity thereto. In embodiments, the mCherry protein is substantially identical to the protein identified by the NCBI reference number GI: 1003312471, or a variant or homolog having substantial identity thereto.

A “OX40” or “OX40 protein” as referred to herein includes any of the recombinant or naturally-occurring forms of tumor necrosis factor receptor superfamily, member 4 (OX40) also known as cluster of differentiation 134 (CD 134) or variants or homologs thereof that maintain OX40 activity (e.g. within at least 50%, 80%, 90%, 95%, 96%, 97%, 98%, 99% or 100% activity compared to OX40). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring OX40 protein. In embodiments, the OX40 protein is substantially identical to the protein identified by the NCBI reference number GI: 32698383, or a variant or homolog having substantial identity thereto. In embodiments, the OX40 protein is substantially identical to the protein identified by the NCBI reference number GI: 732819, or a variant or homolog having substantial identity thereto. In embodiments, the OX40 protein is substantially identical to the protein identified by the NCBI reference number GI: 8926702, or a variant or homolog having substantial identity thereto. In embodiments, the OX40 protein is substantially identical to the protein identified by the NCBI reference number GI: 913406, or a variant or homolog having substantial identity thereto.

The term “OX40 inhibitor” as provided herein refers to a substance (e.g., small molecule, peptide, protein, antibody, antibody fragment, single chain variable fragment [scFv]) capable of detectably lowering expression of or activity level of OX40 compared to a control. The inhibited expression or activity of OX40 can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control. A OX40 inhibitor inhibits OX40 e.g., by at least in part, partially or totally blocking stimulation, decreasing, preventing, or delaying activation, or inactivating, desensitizing, or down-regulating signal transduction, activity or amount of OX40 relative to the absence of the OX40 inhibitor.

A “PD-1” or “PD-1 protein” as referred to herein includes any of the recombinant or naturally-occurring forms of Programmed cell death protein 1 (PD-1) also known as cluster of differentiation 279 (CD 279) or variants or homologs thereof that maintain PD-1 activity (e.g. within at least 50%, 80%, 90%, 95%, 96%, 97%, 98%, 99% or 100% activity compared to PD-1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PD-1 protein. In embodiments, the PD-1 protein is substantially identical to the protein identified by the NCBI reference number GI: 765526769, or a variant or homolog having substantial identity thereto. In embodiments, the PD-1 protein is substantially identical to the protein identified by the NCBI reference number GI: 765526771, or a variant or homolog having substantial identity thereto. In embodiments, the PD-1 protein is substantially identical to the protein identified by the NCBI reference number GI: 765526773, or a variant or homolog having substantial identity thereto. In embodiments, the PD-1 protein is substantially identical to the protein identified by the NCBI reference number GI: 167857792, or a variant or homolog having substantial identity thereto.

A “PD-L1” or “PD-L1 protein” as referred to herein includes any of the recombinant or naturally-occurring forms of programmed death ligand 1 (PD-L1) also known as cluster of differentiation 274 (CD 274) or variants or homologs thereof that maintain PD-L1 activity (e.g. within at least 50%, 80%, 90%, 95%, 96%, 97%, 98%, 99% or 100% activity compared to PD-L1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PD-L1 protein. In embodiments, the PD-L1 protein is substantially identical to the protein identified by the NCBI reference number GI: 119579167, or a variant or homolog having substantial identity thereto. In embodiments, the PD-L1 protein is substantially identical to the protein identified by the NCBI reference number GI: 109731181, or a variant or homolog having substantial identity thereto. In embodiments, the PD-L1 protein is substantially identical to the protein identified by the NCBI reference number GI: 50960360, or a variant or homolog having substantial identity thereto. In embodiments, the PD-L1 protein is substantially identical to the protein identified by the NCBI reference number GI: 46854604, or a variant or homolog having substantial identity thereto.

The term “PD-L1 inhibitor” as provided herein refers to a substance (e.g., small molecule, peptide, protein, antibody, antibody fragment, single chain variable fragment [scFv]) capable of detectably lowering expression of or activity level of PD-L1 compared to a control. The inhibited expression or activity of PD-L1 can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control. A PD-L1 inhibitor inhibits PD-L1 e.g., by at least in part, partially or totally blocking stimulation, decreasing, preventing, or delaying activation, or inactivating, desensitizing, or down-regulating signal transduction, activity or amount of PD-L1 relative to the absence of the PD-L1 inhibitor.

A “PD-1 pathway inhibitor,” “Programmed Death 1 (PD-1) pathway inhibitor,” “PD-1 signaling pathway inhibitor,” or “Programmed Death 1 (PD-1) signaling pathway inhibitor” as provided herein refers to a substance capable of detectably lowering expression of or activity level of the PD-1 signaling pathway compared to a control. The inhibited expression or activity of the PD-1 signaling pathway can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control. An “inhibitor” is a compound or small molecule that inhibits the PD-1 signaling pathway e.g., by binding, partially or totally blocking stimulation of the PD-1 signaling pathway, decrease, prevent, or delay activation of the PD-1 signaling pathway, or inactivate, desensitize, or down-regulate signal transduction, gene expression or enzymatic activity of the PD-1 signaling pathway. The PD-1 pathway inhibitor provided herein may be a PD-1 antagonist or a PD-L1 antagonist. In embodiments, the PD-1 pathway inhibitor is a PD-1 antagonist. Thus, in embodiments, the PD-1 pathway inhibitor inhibits PD-1 activity or expression. In embodiments, the PD-1 pathway inhibitor is a PD-L1 antagonist. Thus, in embodiments, the PD-1 pathway inhibitor inhibits PD-L1 activity or expression. In embodiments, the PD-1 pathway inhibitor is a compound or a small molecule. In embodiments, the PD-1 pathway inhibitor is an antibody.

The term “PD-L1 antagonist” as provided herein refers to a substance capable of detectably lowering expression of or activity level of PD-L1 compared to a control. The inhibited expression or activity of PD-L1 can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control. A PD-L1 antagonist inhibits PD-L1 e.g., by at least in part, partially or totally blocking stimulation, decreasing, preventing, or delaying activation, or inactivating, desensitizing, or down-regulating signal transduction, activity or amount of PD-L1 relative to the absence of the PD-L1 antagonist.

The term “PD-1 antagonist” as provided herein refers to a substance capable of detectably lowering expression of or activity level of PD-1 compared to a control. The inhibited expression or activity of PD-1 can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control. A PD-1 antagonist inhibits PD-1 e.g., by at least in part, partially or totally blocking stimulation, decreasing, preventing, or delaying activation, or inactivating, desensitizing, or down-regulating signal transduction, activity or amount of PD-1 relative to the absence of the PD-1 antagonist.

In embodiments, the PD-1 pathway inhibitor binds to a PD-1 receptor. Thus, the PD-1 pathway inhibitor may be a PD-1 antagonist. In some embodiments, the PD-1 pathway inhibitor is a small molecule inhibitor. In other embodiments, the PD-1 pathway inhibitor is a PD-1 antibody, for example a polyclonal or monoclonal antibody. In one preferred embodiment, the antibody is a monoclonal antibody. Non-limiting examples of suitable PD-1 pathway inhibitors include atezolizumab, nivolumab, pembrolizumab, pidilizumab, avelumab, BMS-936559, AMP-224, durvalumab, avelumab, a biosimilar of any of the foregoing, or any combination of two or more of the foregoing.

In embodiments, the PD-1 pathway inhibitor binds to PD-L1. In embodiments, the PD-1 pathway inhibitor is a PD-L1 antibody. In embodiments, the PD-L1 antibody is avelumab, atezolizumab, durvalumab, or BMS-936559. In embodiments, the PD-1 pathway inhibitor is a compound or a small molecule.

The terms “thymidine kinase gene”, “TK gene”, “TK”, “J2R gene”, or “J2R” as used herein refer to the any of the recombinant or naturally-occurring forms of the thymidine kinase gene or variants or homologs thereof that code for a thymidine kinase polypeptide capable of maintaining the activity of the thymidine kinase polypeptide (e.g., within at least 50%, 80%, 90%, 95%, 96%, 97%, 98%, 99% or 100% activity compared to thymidine kinase polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring thymidine kinase gene. In embodiments, the thymidine kinase gene is substantially identical to the nucleic acid sequence corresponding to position 83422-83955 of the nucleic acid sequence identified by Accession No. DQ121394 or a variant or homolog having substantial identity thereto. In embodiments, the thymidine kinase gene is mutated. In embodiments, the thymidine kinase gene is partially deleted. In embodiments, the thymidine kinase gene includes the nucleic acid sequence of SEQ ID NO:4. In embodiments, the thymidine kinase gene is the nucleic acid sequence of SEQ ID NO:4. In embodiments, the thymidine kinase gene is mutated. In embodiments, the thymidine kinase gene is partially deleted. In embodiments, the thymidine kinase gene includes the nucleic acid sequence of SEQ ID NO:5. In embodiments, the thymidine kinase gene includes the nucleic acid sequence of SEQ ID NO:5.

The term “thymidine kinase” or “thymidine kinase protein” as used herein refers to any of the recombinant or naturally-occurring forms of Thymidine kinase (TK), or variants or homologs thereof that maintain thymidine kinase activity (e.g., within at least 50%, 80%, 90%, 95%, 96%, 97%, 98%, 99% or 100% activity compared to thymidine kinase). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring thymidine kinase protein. In embodiments, the thymidine kinase protein is substantially identical to the protein identified by the NCBI reference number GI: 339719, or a variant or homolog having substantial identity thereto. In embodiments, the thymidine kinase protein is substantially identical to the protein identified by the NCBI reference number GI: 202079, or a variant or homolog having substantial identity thereto. In embodiments, the thymidine kinase protein is substantially identical to the protein identified by the NCBI reference number GI: 25167087, or a variant or homolog having substantial identity thereto.

The term “Human Leukocyte Antigen II” or “Human Leukocyte Antigen II protein” as used herein refers to any of the recombinant or naturally-occurring forms of Human Leukocyte Antigen II (HLA II) also known as MHC class II Human Leukocyte Antigen or Human Leukocyte Antigen class II, or variants or homologs thereof that maintain Human Leukocyte Antigen II activity (e.g., within at least 50%, 80%, 90%, 95%, 96%, 97%, 98%, 99% or 100% activity compared to Human Leukocyte Antigen II). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring Human Leukocyte Antigen II. In embodiments, the Human Leukocyte Antigen II protein is substantially identical to the protein identified by the NCBI reference number GI: 122206, or a variant or homolog having substantial identity thereto. In embodiments, the Human Leukocyte Antigen II protein is substantially identical to the protein identified by the NCBI reference number GI: 451344622, or a variant or homolog having substantial identity thereto. In embodiments, the Human Leukocyte Antigen II protein is substantially identical to the protein identified by the NCBI reference number GI: 290457643, or a variant or homolog having substantial identity thereto. In embodiments, the Human Leukocyte Antigen II protein is substantially identical to the protein identified by the NCBI reference number GI: 545422, or a variant or homolog having substantial identity thereto.

A “CD137” or “CD137 protein” as referred to herein includes any of the recombinant or naturally-occurring forms of tumor necrosis factor receptor superfamily member 9 (TNFRSF9) also known as cluster of differentiation 137 (CD 137), 4-1BB, and induced by lymphocyte activation (ILA) or variants or homologs thereof that maintain CD 137 activity (e.g. within at least 50%, 80%, 90%, 95%, 96%, 97%, 98%, 99% or 100% activity compared to CD 137). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD 137 protein. In embodiments, the CD 137 protein is substantially identical to the protein identified by the NCBI reference number GI: 5730095, or a variant or homolog having substantial identity thereto. In embodiments, the CD 137 protein is substantially identical to the protein identified by the NCBI reference number GI: 119591996, or a variant or homolog having substantial identity thereto. In embodiments, the CD 137 protein is substantially identical to the protein identified by the NCBI reference number GI: 37953283, or a variant or homolog having substantial identity thereto.

The terms “H5 promoter”, “H5”, or the like, as used herein refer to the any of the recombinant or naturally-occurring forms of the H5 promoter or variants or homologs thereof that maintain the activity of the H5 promoter (e.g., within at least 50%, 80%, 90%, 95%, 96%, 97%, 98%, 99% or 100% activity compared to the H5 promoter). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring H5 promoter. In embodiments, the H5 promoter is substantially identical to the nucleic acid sequence corresponding to position 7-76 of the nucleic acid sequence identified by Accession No. FJ386852 or a variant or homolog having substantial identity thereto. In embodiments, the H5 promoter includes the nucleic acid sequence of SEQ ID NO:18. In embodiments, the H5 promoter is the nucleic acid sequence of SEQ ID NO:18.

The terms “SE promoter”, “SE”, or the like, as used herein refer to the any of the recombinant or naturally-occurring forms of the SE promoter or variants or homologs thereof that maintain the activity of the SE promoter (e.g., within at least 50%, 80%, 90%, 95%, 96%, 97%, 98%, 99% or 100% activity compared to the SE promoter). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring SE promoter. In embodiments, the SE promoter includes the nucleic acid sequence of SEQ ID NO:19. In embodiments, the SE promoter is the nucleic acid sequence of SEQ ID NO:19.

The terms “11K promoter”, “11K”, or the like, as used herein refer to the any of the recombinant or naturally-occurring forms of the 11K promoter or variants or homologs thereof that maintain the activity of the 11K promoter (e.g., within at least 50%, 80%, 90%, 95%, 96%, 97%, 98%, 99% or 100% activity compared to the 11K promoter). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring 11K promoter. In embodiments, the 11K promoter is substantially identical to the nucleic acid sequence corresponding to position 40734-40771 of the nucleic acid sequence identified by Accession No. KF179385 or a variant or homolog having substantial identity thereto. In embodiments, the 11K promoter includes the nucleic acid sequence of SEQ ID NO:20. In embodiments, the 11K promoter is the nucleic acid sequence of SEQ ID NO:20.

The terms “virus” or “virus particle” are used according to its plain ordinary meaning within Virology and refers to a virion including the viral genome (e.g. DNA, RNA, single strand, double strand), viral capsid and associated proteins, and in the case of enveloped viruses (e.g. herpesvirus), an envelope including lipids and optionally components of host cell membranes, and/or viral proteins.

The term “poxvirus” is used according to its plain ordinary meaning within Virology and refers to a member of Poxviridae family capable of infecting vertebrates and invertebrates which replicate in the cytoplasm of their host. In embodiments, poxvirus virions have a size of about 200 nm in diameter and about 300 nm in length and possess a genome in a single, linear, double-stranded segment of DNA, typically 130-375 kilobase. The term poxvirus includes, without limitation, all genera of poxviridae (e.g., betaentomopoxvirus, yatapoxvirus, cervidpoxvirus, gammaentomopoxvirus, leporipoxvirus, suipoxvirus, molluscipoxvirus, crocodylidpoxvirus, alphaentomopoxvirus, capripoxvirus, orthopoxvirus, avipoxvirus, and parapoxvirus). In embodiments, the poxvirus is an orthopoxvirus (e.g., smallpox virus, vaccinia virus, cowpox virus, monkeypox virus), parapoxvirus (e.g., orf virus, pseudocowpox virus, bovine popular stomatitis virus), yatapoxvirus (e.g., tanapox virus, yaba monkey tumor virus) or molluscipoxvirus (e.g., molluscum contagiosum virus). In embodiments, the poxvirus is an orthopoxvirus (e.g., cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, or vaccinia virus strain AS). In embodiments, the poxvirus is a parapoxvirus (e.g., orf virus strain NZ2 or pseudocowpox virus strain TJS).

The term “chimeric” used within the context of a chimeric poxvirus, is used according to its plain ordinary meaning within Virology and refers to a hybrid microorganism (e.g., chimeric poxvirus) created by joining nucleic acid fragments from two or more different microorganisms (e.g., two viruses from the same subfamily, two viruses from different subfamilies). In embodiments, the nucleic acid fragments from at least two poxvirus strains combined contain the essential genes necessary for replication. In embodiments, the nucleic acid fragments from one of the at least two poxvirus strains contain the essential genes necessary for replication. The chimeric poxvirus provided herein including embodiments thereof may include one or more transgenes (i.e., nucleic acid sequences not native to the viral genome). For example, the chimeric poxvirus provided herein including embodiments thereof may include an anti-cancer nucleic acid sequence, a nucleic acid binding sequence, a detectable moiety-encoding nucleic acid sequence or any combination thereof. In embodiments, the chimeric poxvirus includes a nucleic acid sequence including an anti-cancer nucleic acid sequence, a nucleic acid binding sequence and a detectable moiety-encoding nucleic acid sequence. In embodiments, the chimeric poxvirus includes a nucleic acid sequence including an anti-cancer nucleic acid sequence and a detectable moiety-encoding nucleic acid sequence. In embodiments, the chimeric poxvirus includes a nucleic acid sequence including a nucleic acid binding sequence and a detectable moiety-encoding nucleic acid sequence. In embodiments, the chimeric poxvirus includes a nucleic acid sequence including an anti-cancer nucleic acid sequence and a nucleic acid binding sequence.

The term “plaque forming units” is used according to its plain ordinary meaning in Virology and refers to a unit of measurement based on the number of plaques per unit volume of a sample. In some embodiments the units are based on the number of plaques that could form when infecting a monolayer of susceptible cells. Plaque forming unit equivalents are units of measure of inactivated virus. In some embodiments, plaque forming unit equivalents are derived from plaque forming units for a sample prior to inactivation. In embodiments, plaque forming units are abbreviated “Pfu”.

The terms “multiplicity of infection” or “MOI” are used according to its plain ordinary meaning in Virology and refers to the ratio of components (e.g., poxvirus) to the target (e.g., cell) in a given area. In embodiments, the area is assumed to be homogenous.

The term “replicate” is used in accordance with its plain ordinary meaning and refers to the ability of a cell or virus to produce progeny. A person of ordinary skill in the art will immediately understand that the term replicate when used in connection with DNA, refers to the biological process of producing two identical replicas of DNA from one original DNA molecule. In the context of a virus, the term “replicate” includes the ability of a virus to replicate (duplicate the viral genome and packaging said genome into viral particles) in a host cell and subsequently release progeny viruses from the host cell, which results in the lysis of the host cell. A “replication-competent” virus as provided herein refers to a virus (chimeric poxvirus) that is capable of replicating in a cell (e.g., a cancer cell). Similarly, an “oncolytic virus” as referred to herein, is a virus that is capable of infecting and killing cancer cells. As the infected cancer cells are destroyed by oncolysis, they release new infectious virus particles or virions to help destroy the remaining tumor. In embodiments, the chimeric poxvirus is able to replicate in a cancer cell. In embodiments, the chimeric poxvirus does not detectably replicate in a healthy cell relative to a standard control. In embodiments, the chimeric poxvirus provided herein has an increased oncolytic activity compared to its parental virus. In embodiments, the oncolytic activity (ability to induce cell death in an infected cell) is more than 1.5, 2, 3, 4, 5, 6, 7, 8, 9, 10, 100, 10000, 10000 times increased compared to the oncolytic activity of a parental virus (one of the viruses used to form the chimeric virus provided herein).

The term “cowpox virus strain Brighton” is used according to its common, ordinary meaning and refers to virus strains of the same or similar names and functional fragments and homologs thereof. The term includes recombinant or naturally occurring forms of cowpox virus strain Brighton or variants thereof that maintain cowpox virus strain Brighton activity (e.g. within at least 30%, 40%, 50%, 60%, 70%, 80%, 90%, 95%, or 100%). The term includes recombinant or naturally occurring forms of cowpox virus strain Brighton or variants thereof whose genome has sequence identity to the cowpox virus strain Brighton genome (e.g. about 65%, 70%, 75%, 80%, 85%, 90%, 95%, 99% or 100% identity to the cowpox virus strain Brighton genome). Cowpox virus strain Brighton may refer to variants having mutated amino acid residues that modulate (e.g. increase or decrease when compared to cowpox virus strain Brighton) cowpox virus strain Brighton activity, expression, cellular targeting, or infectivity. Cowpox virus strain Brighton may be modified as described herein. In embodiments, the cowpox virus strain Brighton refers to the virus strain identified by ATCC (American Type Culture Collection) reference number ATCC VR-302™, variants or homologs thereof. In embodiments, the cowpox virus strain Brighton refers to the virus strain identified by Taxonomy reference number 265872, variants or homologs thereof.

The term “raccoonpox virus strain Herman” is used according to its common, ordinary meaning and refers to virus strains of the same or similar names and functional fragments and homologs thereof. The term includes recombinant or naturally occurring forms of raccoonpox virus strain Herman or variants thereof that maintain raccoonpox virus strain Herman activity (e.g. within at least 30%, 40%, 50%, 60%, 70%, 80%, 90%, 95%, or 100%). The term includes recombinant or naturally occurring forms of raccoonpox virus strain Herman or variants thereof whose genome has sequence identity to the raccoonpox virus strain Herman genome (e.g. about 65%, 70%, 75%, 80%, 85%, 90%, 95%, 99% or 100% identity to the raccoonpox virus strain Herman genome). Raccoonpox virus strain Herman may refer to variants having mutated amino acid residues that modulate (e.g. increase or decrease when compared to raccoonpox virus strain Herman) raccoonpox virus strain Herman activity, expression, cellular targeting, or infectivity. Raccoonpox virus strain Herman may be modified as described herein. In embodiments, the raccoonpox virus strain Herman refers to the virus strain identified by ATCC reference number ATCC VR-838™, variants or homologs thereof. In embodiments, the raccoonpox virus strain Herman refers to the virus strain encoded by the nucleic acid sequence with the reference number NC_027213.

The term “rabbitpox virus strain Utrecht” is used according to its common, ordinary meaning and refers to virus strains of the same or similar names and functional fragments and homologs thereof. The term includes recombinant or naturally occurring forms of rabbitpox virus strain Utrecht or variants thereof that maintain rabbitpox virus strain Utrecht activity (e.g. within at least 30%, 40%, 50%, 60%, 70%, 80%, 90%, 95%, or 100%). The term includes recombinant or naturally occurring forms of rabbitpox virus strain Utrecht or variants thereof whose genome has sequence identity to the rabbitpox virus strain Utrecht genome (e.g. about 65%, 70%, 75%, 80%, 85%, 90%, 95%, 99% or 100% identity to the rabbitpox virus strain Utrecht genome). Rabbitpox virus strain Utrecht may refer to variants having mutated amino acid residues that modulate (e.g. increase or decrease when compared to rabbitpox virus strain Utrecht) rabbitpox virus strain Utrecht activity, expression, cellular targeting, or infectivity. Rabbitpox virus strain Utrecht may be modified as described herein. In embodiments, the rabbitpox virus strain Utrecht refers to the virus strain identified by ATCC reference number ATCC VR-1591™, variants or homologs thereof. In embodiments, the rabbitpox virus strain Utrecht refers to the virus strain identified by Taxonomy reference number 45417, variants or homologs thereof.

The term “vaccinia virus strain WR” is used according to its common, ordinary meaning and refers to virus strains of the same or similar names and functional fragments and homologs thereof. The term includes recombinant or naturally occurring forms of vaccinia virus strain WR or variants thereof that maintain vaccinia virus strain WR activity (e.g. within at least 30%, 40%, 50%, 60%, 70%, 80%, 90%, 95%, or 100%). The term includes recombinant or naturally occurring forms of vaccinia virus strain WR or variants thereof whose genome has sequence identity to the vaccinia virus strain WR genome (e.g. about 65%, 70%, 75%, 80%, 85%, 90%, 95%, 99% or 100% identity to the vaccinia virus strain WR genome). Vaccinia virus strain WR may refer to variants having mutated amino acid residues that modulate (e.g. increase or decrease when compared to vaccinia virus strain WR) vaccinia virus strain WR activity, expression, cellular targeting, or infectivity. Vaccinia virus strain WR may be modified as described herein. In embodiments, the vaccinia virus strain WR refers to the virus strain identified by ATCC reference number ATCC VR-1354™, variants or homologs thereof. In embodiments, the vaccinia virus strain WR refers to the virus strain identified by Taxonomy reference number 10254, variants or homologs thereof.

The term “vaccinia virus strain IHD” is used according to its common, ordinary meaning and refers to virus strains of the same or similar names and functional fragments and homologs thereof. The term includes recombinant or naturally occurring forms of vaccinia virus strain IHD or variants thereof that maintain vaccinia virus strain IHD activity (e.g. within at least 30%, 40%, 50%, 60%, 70%, 80%, 90%, 95%, or 100%). The term includes recombinant or naturally occurring forms of vaccinia virus strain IHD or variants thereof whose genome has sequence identity to the vaccinia virus strain IHD genome (e.g. about 65%, 70%, 75%, 80%, 85%, 90%, 95%, 99% or 100% identity to the vaccinia virus strain IHD genome). Vaccinia virus strain IHD may refer to variants having mutated amino acid residues that modulate (e.g. increase or decrease when compared to vaccinia virus strain IHD) vaccinia virus strain IHD activity, expression, cellular targeting, or infectivity. Vaccinia virus strain IHD may be modified as described herein. In embodiments, the vaccinia virus strain IHD refers to the virus strain identified by ATCC reference number ATCC VR-156™, variants or homologs thereof. In embodiments, the vaccinia virus strain IHD refers to the virus strain identified by Taxonomy reference number 10251, variants or homologs thereof.

The term “vaccinia virus strain Elstree” is used according to its common, ordinary meaning and refers to virus strains of the same or similar names and functional fragments and homologs thereof. The term includes recombinant or naturally occurring forms of vaccinia virus strain Elstree or variants thereof that maintain vaccinia virus strain Elstree activity (e.g. within at least 30%, 40%, 50%, 60%, 70%, 80%, 90%, 95%, or 100%). The term includes recombinant or naturally occurring forms of vaccinia virus strain Elstree or variants thereof whose genome has sequence identity to the vaccinia virus strain Elstree genome (e.g. about 65%, 70%, 75%, 80%, 85%, 90%, 95%, 99% or 100% identity to the vaccinia virus strain Elstree genome). Vaccinia virus strain Elstree may refer to variants having mutated amino acid residues that modulate (e.g. increase or decrease when compared to vaccinia virus strain Elstre) vaccinia virus strain Elstree activity, expression, cellular targeting, or infectivity. Vaccinia virus strain Elstree may be modified as described herein. In embodiments, the vaccinia virus strain Elstree refers to the virus strain identified by ATCC reference number ATCC VR-1549™, variants or homologs thereof.

The term “vaccinia virus strain CL” is used according to its common, ordinary meaning and refers to virus strains of the same or similar names and functional fragments and homologs thereof. The term includes recombinant or naturally occurring forms of vaccinia virus strain CL or variants thereof that maintain vaccinia virus strain CL activity (e.g. within at least 30%, 40%, 50%, 60%, 70%, 80%, 90%, 95%, or 100%). The term includes recombinant or naturally occurring forms of vaccinia virus strain CL or variants thereof whose genome has sequence identity to the vaccinia virus strain CL genome (e.g. about 65%, 70%, 75%, 80%, 85%, 90%, 95%, 99% or 100% identity to the vaccinia virus strain CL genome). Vaccinia virus strain CL may refer to variants having mutated amino acid residues that modulate (e.g. increase or decrease when compared to vaccinia virus strain CL) vaccinia virus strain CL activity, expression, cellular targeting, or infectivity. Vaccinia virus strain CL may be modified as described herein. In embodiments, the vaccinia virus strain CL refers to the virus strain identified by ATCC reference number ATCC VR-1774™, variants or homologs thereof.

The term “vaccinia virus strain Lederle-Chorioallantoic” is used according to its common, ordinary meaning and refers to virus strains of the same or similar names and functional fragments and homologs thereof. The term includes recombinant or naturally occurring forms of vaccinia virus strain Lederle-Chorioallantoic or variants thereof that maintain vaccinia virus strain Lederle-Chorioallantoic activity (e.g. within at least 30%, 40%, 50%, 60%, 70%, 80%, 90%, 95%, or 100%). The term includes recombinant or naturally occurring forms of vaccinia virus strain Lederle-Chorioallantoic or variants thereof whose genome has sequence identity to the vaccinia virus strain Lederle-Chorioallantoic genome (e.g. about 65%, 70%, 75%, 80%, 85%, 90%, 95%, 99% or 100% identity to the vaccinia virus strain Lederle-Chorioallantoic genome). Vaccinia virus strain Lederle-Chorioallantoic may refer to variants having mutated amino acid residues that modulate (e.g. increase or decrease when compared to vaccinia virus strain Lederle-Chorioallantoic) vaccinia virus strain Lederle-Chorioallantoic activity, expression, cellular targeting, or infectivity. Vaccinia virus strain Lederle-Chorioallantoic may be modified as described herein. In embodiments, the vaccinia virus strain Lederle-Chorioallantoic refers to the virus strain identified by ATCC reference number ATCC VR-118™, variants or homologs thereof.

The term “vaccinia virus strain AS” is used according to its common, ordinary meaning and refers to virus strains of the same or similar names and functional fragments and homologs thereof. The term includes recombinant or naturally occurring forms of vaccinia virus strain AS or variants thereof that maintain vaccinia virus strain AS activity (e.g. within at least 30%, 40%, 50%, 60%, 70%, 80%, 90%, 95%, or 100%). The term includes recombinant or naturally occurring forms of vaccinia virus strain AS or variants thereof whose genome has sequence identity to the vaccinia virus strain AS genome (e.g. about 65%, 70%, 75%, 80%, 85%, 90%, 95%, 99% or 100% identity to the vaccinia virus strain AS genome). Vaccinia virus strain AS may refer to variants having mutated amino acid residues that modulate (e.g. increase or decrease when compared to vaccinia virus strain AS) vaccinia virus strain AS activity, expression, cellular targeting, or infectivity. Vaccinia virus strain AS may be modified as described herein. In embodiments, the vaccinia virus strain AS refers to the virus strain identified by ATCC reference number ATCC VR-2010™, variants or homologs thereof.

The term “orf virus strain NZ2” is used according to its common, ordinary meaning and refers to virus strains of the same or similar names and functional fragments and homologs thereof. The term includes recombinant or naturally occurring forms of orf virus strain NZ2 or variants thereof that maintain orf virus strain NZ2 activity (e.g. within at least 30%, 40%, 50%, 60%, 70%, 80%, 90%, 95%, or 100%). The term includes recombinant or naturally occurring forms of orf virus strain NZ2 or variants thereof whose genome has sequence identity to the orf virus strain NZ2 genome (e.g. about 65%, 70%, 75%, 80%, 85%, 90%, 95%, 99% or 100% identity to the orf virus strain NZ2 genome). Orf virus strain NZ2 may refer to variants having mutated amino acid residues that modulate (e.g. increase or decrease when compared to orf virus strain NZ2) orf virus strain NZ2 activity, expression, cellular targeting, or infectivity. Orf virus strain NZ2 may be modified as described herein. In embodiments, the orf virus strain NZ2 refers to the virus strain identified by ATCC reference number ATCC VR-1548™, variants or homologs thereof. In embodiments, the orf virus strain NZ2 refers to the virus strain identified by Taxonomy reference number 10259, variants or homologs thereof.

The term “pseudocowpox virus strain TJS” is used according to its common, ordinary meaning and refers to virus strains of the same or similar names and functional fragments and homologs thereof. The term includes recombinant or naturally occurring forms of pseudocowpox virus strain TJS or variants thereof that maintain pseudocowpox virus strain TJS activity (e.g. within at least 30%, 40%, 50%, 60%, 70%, 80%, 90%, 95%, or 100%). The term includes recombinant or naturally occurring forms of pseudocowpox virus strain TJS or variants thereof whose genome has sequence identity to the pseudocowpox virus strain TJS genome (e.g. about 65%, 70%, 75%, 80%, 85%, 90%, 95%, 99% or 100% identity to the pseudocowpox virus strain TJS genome). Pseudocowpox virus strain TJS may refer to variants having mutated amino acid residues that modulate (e.g. increase or decrease when compared to pseudocowpox virus strain TJS) pseudocowpox virus strain TJS activity, expression, cellular targeting, or infectivity. Pseudocowpox virus strain TJS may be modified as described herein. In embodiments, the pseudocowpox virus strain TJS refers to the virus strain identified by ATCC reference number ATCC VR-634™, variants or homologs thereof.

In embodiments, cowpox virus strain Brighton is cowpox virus strain Brighton ATCC VR-302™. In embodiments, raccoonpox virus strain Herman is raccoonpox virus strain Herman ATCC VR-838™. In embodiments, rabbitpox virus strain Utrecht is rabbitpox virus strain Utrecht ATCC VR-1591™. In embodiments, vaccinia virus strain WR is vaccinia virus strain WR ATCC VR-1354™. In embodiments, vaccinia virus strain IHD is vaccinia virus strain IHD ATCC VR-156™. In embodiments, vaccinia virus strain Elstree is vaccinia virus strain Elstree ATCC VR-1549™. In embodiments, vaccinia virus strain CL is vaccinia virus strain CL ATCC VR-1774™. In embodiments, vaccinia virus strain Lederle-Chorioallantoic is vaccinia virus strain Lederle-Chorioallantoic ATCC VR-118™. In embodiments, vaccinia virus strain AS is vaccinia virus strain AS ATCC VR-2010™. In embodiments, orf virus strain NZ2 is orf virus strain NZ2 ATCC VR-1548™. In embodiments, pseudocowpox virus strain TJS is pseudocowpox virus strain TJS ATCC VR-634™. In embodiments, the cowpox virus strain Brighton refers to the virus strain identified by Taxonomy reference number 265872, variants or homologs thereof.

In this disclosure, “comprises,” “comprising,” “containing” and “having” and the like can have the meaning ascribed to them in U.S. Patent law and can mean “includes,” “including,” and the like. “Consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

A “synergistic amount” as used herein refers to the sum of a first amount (e.g., an amount of a compound provided herein) and a second amount (e.g., a therapeutic agent) that results in a synergistic effect (i.e. an effect greater than an additive effect). Therefore, the terms “synergy”, “synergism”, “synergistic”, “combined synergistic amount”, and “synergistic therapeutic effect” which are used herein interchangeably, refer to a measured effect of the compound administered in combination where the measured effect is greater than the sum of the individual effects of each of the compounds provided herein administered alone as a single agent.

In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the compound provided herein when used separately from the therapeutic agent. In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the therapeutic agent when used separately from the compound provided herein.

The term “EC50” or “half maximal effective concentration” as used herein refers to the concentration of a molecule (e.g., antibody, chimeric antigen receptor or bispecific antibody) capable of inducing a response which is halfway between the baseline response and the maximum response after a specified exposure time. In embodiments, the EC50 is the concentration of a molecule (e.g., antibody, chimeric antigen receptor or bispecific antibody) that produces 50% of the maximal possible effect of that molecule.

A “therapeutic agent” as used herein refers to an agent (e.g., compound or composition described herein) that when administered to a subject will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms or the intended therapeutic effect, e.g., treatment or amelioration of an injury, disease, pathology or condition, or their symptoms including any objective or subjective parameter of treatment such as abatement; remission; diminishing of symptoms or making the injury, pathology or condition more tolerable to the patient; slowing in the rate of degeneration or decline; making the final point of degeneration less debilitating; or improving a patient's physical or mental well-being.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

A “effective amount” is an amount sufficient for a compound (e.g., a neural stem cell including a chimeric poxvirus) to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound (e.g., a neural stem cell including a chimeric poxvirus) described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent (e.g., a neural stem cell including a chimeric poxvirus) sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound (e.g., a neural stem cell including a chimeric poxvirus) being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

Certain compounds (e.g., a neural stem cell including a chimeric poxvirus) of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

A “stem cell” is a cell characterized by the ability of self-renewal through mitotic cell division and the potential to differentiate into a tissue or an organ. Among mammalian stem cells, embryonic stem cells (ES cells) and somatic stem cells (e.g., HSC) can be distinguished. Embryonic stem cells reside in the blastocyst and give rise to embryonic tissues, whereas somatic stem cells reside in adult tissues for the purpose of tissue regeneration and repair. A “neural stem cell” as provided herein refers to a stem cell capable to self-renew through mitotic cell division and to differentiate into a neural cell (e.g., glia cell, neuron, astrocyte, oligodendrocyte).

The term “culture” or “cell culture” means the maintenance of cells, for example cancer cells, in an artificial, in vitro environment. A “cell culture system” is used herein to refer to culture conditions in which a population of cells may be grown as monolayers or in suspension. “Culture medium” is used herein to refer to a nutrient solution for the culturing, growth, or proliferation of cells.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples). One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

The terms “disease” or “condition” refer to a state of being or health status of a patient or subject capable of being treated with the compounds or methods provided herein. The disease may be a cancer. In some further instances, “cancer” refers to human cancers and carcinomas, sarcomas, adenocarcinomas, lymphomas, leukemias, including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “cancer” refers to all types of cancer, neoplasm or malignant tumors found in mammals (e.g. humans), including leukemia, carcinomas and sarcomas. Exemplary cancers that may be treated with a compound or method provided herein include breast cancer, colon cancer, kidney cancer, leukemia, lung cancer, melanoma, ovarian cancer, prostate cancer, pancreatic cancer, brain cancer, liver cancer, gastric cancer or a sarcoma.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the interal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

“Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. Examplary anti-cancer agents include antibodies, small molecules, large molecules, and combinations thereof. In embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds or platinum containing agents (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or r1L₂), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), Vincristine sulfate, Cryptophycin 52 (i.e. LY-355703), Vitilevuamide, Tubulysin A, Canadensol, Centaureidin (i.e. NSC-106969), Oncocidin A1 (i.e. BTO-956 and DIME), Fijianolide B, Laulimalide, Narcosine (also known as NSC-5366), Nascapine, Hemiasterlin, Vanadocene acetylacetonate, Monsatrol, lnanocine (i.e. NSC-698666), Eleutherobins (such as Desmethyleleutherobin, Desaetyleleutherobin, lsoeleutherobin A, and Z-Eleutherobin), Caribaeoside, Caribaeolin, Halichondrin B, Diazonamide A, Taccalonolide A, Diozostatin, (−)-Phenylahistin (i.e. NSCL-96F037), Myoseverin B, Resverastatin phosphate sodium, steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, hormonal therapies, or the like. Cancer model organism, as used herein, is an organism exhibiting a phenotype indicative of cancer, or the activity of cancer causing elements, within the organism. The term cancer is defined above. A wide variety of organisms may serve as cancer model organisms, and include for example, cancer cells and mammalian organisms such as rodents (e.g. mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g. mice) and from humans.

As defined herein the terms “immune checkpoint”, “immune checkpoint protein” or “checkpoint protein” may be used interchangeably and refer to compositions (molecules) capable of modulating the duration and amplitude of physiological immune responses (e.g., attenuate and/or eliminate sustained immune cell activation, hus regulating normal immune homeostasis). Immune checkpoint proteins may stimulate (increase) an immune response. In embodiments, the checkpoint protein is a cellular receptor. Examples, of stimulatory checkpoint molecules include, but are not limited to, members of the tumor necrosis factor (TNF) receptor superfamily (e.g. CD27, CD40, OX40, glucocorticoid-induced TNFR family related gene (GITR), and CD137), members of the B7-CD28 superfamily (e.g. CD28 itself and Inducible T-cell costimulator (ICOS)). Alternatively, immune checkpoint proteins may inhibit (decrease) an immune response. Examples of inhibitory checkpoint molecules include, but are not limited to, adenosine A2A receptor (A2AR), B7-H3, B7-H4, BTLA, CTLA-4, indoleamine 2,3-dioxygenase (IDO), killer immunoglobulin-like receptors (KIR), LAG3, PD-1, TIM-3, and V-domain immunoglobulin suppressor of T-cell activation (VISTA) protein.

Likewise, an “immune checkpoint inhibitor” or “checkpoint inhibitor” as provided herein refers to a substance (e.g., an antibody or fragment thereof, a small molecule) that is capable of inhibiting, negatively affecting (e.g., decreasing) the activity or function of a checkpoint protein (e.g., decreasing expression or decreasing the activity of a checkpoint protein) relative to the activity or function of the checkpoint protein in the absence of the inhibitor. The checkpoint inhibitor may at least in part, partially or totally block stimulation, decrease, prevent, or delay activation, or inactivate, desensitize, or down-regulate signal transduction or enzymatic activity or the amount of a checkpoint protein. A checkpoint inhibitor may inhibit a checkpoint protein, e.g., by binding, partially or totally blocking, decreasing, preventing, delaying, inactivating, desensitizing, or down-regulating activity of the checkpoint protein. In embodiments, the checkpoint inhibitor is an antibody. In embodiments, the checkpoint inhibitor is an antibody fragment. In embodiments, the checkpoint inhibitor is an antibody variant. In embodiments, the checkpoint inhibitor is a scFv. In embodiments, the checkpoint inhibitor is an anti-CTLA-4 antibody. In embodiments, the checkpoint inhibitor is an anti-PD1 antibody. In embodiments, the checkpoint inhibitor is an anti-PD-L1 antibody. In embodiments, the checkpoint inhibitor is an anti-LAG-3 antibody. In embodiments, the checkpoint inhibitor is an anti-IgG1k antibody. In embodiments, the checkpoint inhibitor is an anti-CD25 antibody. In embodiments, the checkpoint inhibitor is an anti-IL2R antibody. In embodiments, the checkpoint inhibitor forms part of an oncolytic virus. Non-limiting examples of checkpoint inhibitors include ipilimumab, pembrolizumab, nivolumab, talimogene laherparepvec, durvalumab, daclizumab, avelumab, and atezolizumab.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

As defined herein, the term “activation”, “activate”, “activating”, “activator” and the like in reference to a protein-inhibitor interaction means positively affecting (e.g. increasing) the activity or function of the protein relative to the activity or function of the protein in the absence of the activator. In embodiments activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g. a protein associated disease, a cancer (e.g., cancer, inflammatory disease, autoimmune disease, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The term “diagnosis” refers to a relative probability that a disease (e.g. cancer) is present in the subject. Similarly, the term “prognosis” refers to a relative probability that a certain future outcome may occur in the subject with respect to a disease state. For example, in the context of the present invention, prognosis can refer to the likelihood that an individual will develop a disease (e.g. cancer), or the likely severity of the disease (e.g., duration of disease). The terms are not intended to be absolute, as will be appreciated by any one of skill in the field of medical diagnostics.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

In this disclosure, “comprises,” “comprising,” “containing” and “having” and the like can have the meaning ascribed to them in U.S. patent law and can mean “includes,” “including,” and the like. “Consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

### II. Viral Compositions

Provided herein are compositions and methods, that are, inter alia, useful for the treatment of cancer (e.g., ovarian cancer, metastatic ovarian cancer). The compositions provided herein include neural stem cells including chimeric poxviruses. Where the neural stem cell includes a chimeric poxvirus, the neural stem cell includes one or more copies of the genome of the chimeric poxvirus, chimeric poxvirus particles or a combination thereof. The neural stem cell provided herein may be used for targeted delivery of the chimeric poxvirus to the site of a cancer or metastatic cells of such cancer. Upon delivery of the chimeric poxvirus to the cancer site by said neural stem cell the chimeric poxvirus is able to specifically infect the cancer cell, replicate in said cancer cell and upon viral release lyse said cancer cell. The chimeric poxvirus replicates selectively within cancer cells until they lyse and release the chimeric poxvirus to surrounding cancer cells, amplifying its therapeutic effect until normal cells (healthy cells) are reached. In embodiments, the chimeric poxvirus is CF33. CF33 as referred to herein is a chimeric poxvirus encoded by a nucleic acid sequence having a sequence of SEQ ID NO:1. CF17 as referred to herein is a chimeric poxvirus encoded by a nucleic acid sequence having a sequence of SEQ ID NO:2.

### Chimeric Poxviruses:

In embodiments, the chimeric poxvirus is encoded by a nucleic acid sequence having a sequence identity of at least 70% to SEQ ID NO:1. In embodiments, the nucleic acid sequence has a sequence identity of at least 71% to SEQ ID NO:1. In embodiments, the nucleic acid sequence has a sequence identity of at least 72% to SEQ ID NO:1. In embodiments, the nucleic acid sequence has a sequence identity of at least 73% to SEQ ID NO:1. In embodiments, the nucleic acid sequence has a sequence identity of at least 74% to SEQ ID NO:1. In embodiments, the nucleic acid sequence has a sequence identity of at least 75% to SEQ ID NO:1. In embodiments, the nucleic acid sequence has a sequence identity of at least 76% to SEQ ID NO:1. In embodiments, the nucleic acid sequence has a sequence identity of at least 77% to SEQ ID NO:1. In embodiments, the nucleic acid sequence has a sequence identity of at least 78% to SEQ ID NO:1. In embodiments, the nucleic acid sequence has a sequence identity of at least 79% to SEQ ID NO:1. In embodiments, the nucleic acid sequence has a sequence identity of at least 80% to SEQ ID NO:1.

In embodiments, the nucleic acid sequence has a sequence identity of at least 81% to SEQ ID NO:1. In embodiments, the nucleic acid sequence has a sequence identity of at least 82% to SEQ ID NO:1. In embodiments, the nucleic acid sequence has a sequence identity of at least 83% to SEQ ID NO:1. In embodiments, the nucleic acid sequence has a sequence identity of at least 84% to SEQ ID NO:1. In embodiments, the nucleic acid sequence has a sequence identity of at least 85% to SEQ ID NO:1. In embodiments, the nucleic acid sequence has a sequence identity of at least 86% to SEQ ID NO:1. In embodiments, the nucleic acid sequence has a sequence identity of at least 87% to SEQ ID NO:1. In embodiments, the nucleic acid sequence has a sequence identity of at least 88% to SEQ ID NO:1. In embodiments, the nucleic acid sequence has a sequence identity of at least 89% to SEQ ID NO:1. In embodiments, the nucleic acid sequence has a sequence identity of at least 90% to SEQ ID NO:1.

In embodiments, the nucleic acid sequence has a sequence identity of at least 91% to SEQ ID NO:1. In embodiments, the nucleic acid sequence has a sequence identity of at least 92% to SEQ ID NO:1. In embodiments, the nucleic acid sequence has a sequence identity of at least 93% to SEQ ID NO:1. In embodiments, the nucleic acid sequence has a sequence identity of at least 94% to SEQ ID NO:1. In embodiments, the nucleic acid sequence has a sequence identity of at least 95% to SEQ ID NO:1. In embodiments, the nucleic acid sequence has a sequence identity of at least 96% to SEQ ID NO:1. In embodiments, the nucleic acid sequence has a sequence identity of at least 97% to SEQ ID NO:1. In embodiments, the nucleic acid sequence has a sequence identity of at least 98% to SEQ ID NO:1. In embodiments, the nucleic acid sequence has a sequence identity of at least 99% to SEQ ID NO:1.

In embodiments, the nucleic acid sequence has a sequence identity of at least 71% to SEQ ID NO:2. In embodiments, the nucleic acid sequence has a sequence identity of at least 72% to SEQ ID NO:2. In embodiments, the nucleic acid sequence has a sequence identity of at least 73% to SEQ ID NO:2. In embodiments, the nucleic acid sequence has a sequence identity of at least 74% to SEQ ID NO:2. In embodiments, the nucleic acid sequence has a sequence identity of at least 75% to SEQ ID NO:2. In embodiments, the nucleic acid sequence has a sequence identity of at least 76% to SEQ ID NO:2. In embodiments, the nucleic acid sequence has a sequence identity of at least 77% to SEQ ID NO:2. In embodiments, the nucleic acid sequence has a sequence identity of at least 78% to SEQ ID NO:2. In embodiments, the nucleic acid sequence has a sequence identity of at least 79% to SEQ ID NO:2. In embodiments, the nucleic acid sequence has a sequence identity of at least 80% to SEQ ID NO:2.

In embodiments, the nucleic acid sequence has a sequence identity of at least 81% to SEQ ID NO:2. In embodiments, the nucleic acid sequence has a sequence identity of at least 82% to SEQ ID NO:2. In embodiments, the nucleic acid sequence has a sequence identity of at least 83% to SEQ ID NO:2. In embodiments, the nucleic acid sequence has a sequence identity of at least 84% to SEQ ID NO:2. In embodiments, the nucleic acid sequence has a sequence identity of at least 85% to SEQ ID NO:2. In embodiments, the nucleic acid sequence has a sequence identity of at least 86% to SEQ ID NO:2. In embodiments, the nucleic acid sequence has a sequence identity of at least 87% to SEQ ID NO:2. In embodiments, the nucleic acid sequence has a sequence identity of at least 88% to SEQ ID NO:2. In embodiments, the nucleic acid sequence has a sequence identity of at least 89% to SEQ ID NO:2. In embodiments, the nucleic acid sequence has a sequence identity of at least 90% to SEQ ID NO:2.

In embodiments, the nucleic acid sequence has a sequence identity of at least 91% to SEQ ID NO:2. In embodiments, the nucleic acid sequence has a sequence identity of at least 92% to SEQ ID NO:2. In embodiments, the nucleic acid sequence has a sequence identity of at least 93% to SEQ ID NO:2. In embodiments, the nucleic acid sequence has a sequence identity of at least 94% to SEQ ID NO:2. In embodiments, the nucleic acid sequence has a sequence identity of at least 95% to SEQ ID NO:2. In embodiments, the nucleic acid sequence has a sequence identity of at least 96% to SEQ ID NO:2. In embodiments, the nucleic acid sequence has a sequence identity of at least 97% to SEQ ID NO:2. In embodiments, the nucleic acid sequence has a sequence identity of at least 98% to SEQ ID NO:2. In embodiments, the nucleic acid sequence has a sequence identity of at least 99% to SEQ ID NO:2.

In embodiments, the nucleic acid sequence has a sequence identity of 71% to SEQ ID NO:1. In embodiments, the nucleic acid sequence has a sequence identity of 72% to SEQ ID NO:1. In embodiments, the nucleic acid sequence has a sequence identity of 73% to SEQ ID NO:1. In embodiments, the nucleic acid sequence has a sequence identity of 74% to SEQ ID NO:1. In embodiments, the nucleic acid sequence has a sequence identity of 75% to SEQ ID NO:1. In embodiments, the nucleic acid sequence has a sequence identity of 76% to SEQ ID NO:1. In embodiments, the nucleic acid sequence has a sequence identity of 77% to SEQ ID NO:1. In embodiments, the nucleic acid sequence has a sequence identity of 78% to SEQ ID NO:1. In embodiments, the nucleic acid sequence has a sequence identity of 79% to SEQ ID NO:1. In embodiments, the nucleic acid sequence has a sequence identity of 80% to SEQ ID NO:1.

In embodiments, the nucleic acid sequence has a sequence identity of 81% to SEQ ID NO:1. In embodiments, the nucleic acid sequence has a sequence identity of 82% to SEQ ID NO:1. In embodiments, the nucleic acid sequence has a sequence identity of 83% to SEQ ID NO:1. In embodiments, the nucleic acid sequence has a sequence identity of 84% to SEQ ID NO:1. In embodiments, the nucleic acid sequence has a sequence identity of 85% to SEQ ID NO:1. In embodiments, the nucleic acid sequence has a sequence identity of 86% to SEQ ID NO:1. In embodiments, the nucleic acid sequence has a sequence identity of 87% to SEQ ID NO:1. In embodiments, the nucleic acid sequence has a sequence identity of 88% to SEQ ID NO:1. In embodiments, the nucleic acid sequence has a sequence identity of 89% to SEQ ID NO:1. In embodiments, the nucleic acid sequence has a sequence identity of 90% to SEQ ID NO:1.

In embodiments, the nucleic acid sequence has a sequence identity of 91% to SEQ ID NO:1. In embodiments, the nucleic acid sequence has a sequence identity of 92% to SEQ ID NO:1. In embodiments, the nucleic acid sequence has a sequence identity of 93% to SEQ ID NO:1. In embodiments, the nucleic acid sequence has a sequence identity of 94% to SEQ ID NO:1. In embodiments, the nucleic acid sequence has a sequence identity of 95% to SEQ ID NO:1. In embodiments, the nucleic acid sequence has a sequence identity of 96% to SEQ ID NO:1. In embodiments, the nucleic acid sequence has a sequence identity of 97% to SEQ ID NO:1. In embodiments, the nucleic acid sequence has a sequence identity of 98% to SEQ ID NO:1. In embodiments, the nucleic acid sequence has a sequence identity of 99% to SEQ ID NO:1. In embodiments, the nucleic acid sequence is the sequence of SEQ ID NO:1.

In embodiments, the nucleic acid sequence has a sequence identity of 71% to SEQ ID NO:2. In embodiments, the nucleic acid sequence has a sequence identity of 72% to SEQ ID NO:2. In embodiments, the nucleic acid sequence has a sequence identity of 73% to SEQ ID NO:2. In embodiments, the nucleic acid sequence has a sequence identity of 74% to SEQ ID NO:2. In embodiments, the nucleic acid sequence has a sequence identity of 75% to SEQ ID NO:2. In embodiments, the nucleic acid sequence has a sequence identity of 76% to SEQ ID NO:2. In embodiments, the nucleic acid sequence has a sequence identity of 77% to SEQ ID NO:2. In embodiments, the nucleic acid sequence has a sequence identity of 78% to SEQ ID NO:2. In embodiments, the nucleic acid sequence has a sequence identity of 79% to SEQ ID NO:2. In embodiments, the nucleic acid sequence has a sequence identity of 80% to SEQ ID NO:2.

In embodiments, the nucleic acid sequence has a sequence identity of 81% to SEQ ID NO:2. In embodiments, the nucleic acid sequence has a sequence identity of 82% to SEQ ID NO:2. In embodiments, the nucleic acid sequence has a sequence identity of 83% to SEQ ID NO:2. In embodiments, the nucleic acid sequence has a sequence identity of 84% to SEQ ID NO:2. In embodiments, the nucleic acid sequence has a sequence identity of 85% to SEQ ID NO:2. In embodiments, the nucleic acid sequence has a sequence identity of 86% to SEQ ID NO:2. In embodiments, the nucleic acid sequence has a sequence identity of 87% to SEQ ID NO:2. In embodiments, the nucleic acid sequence has a sequence identity of 88% to SEQ ID NO:2. In embodiments, the nucleic acid sequence has a sequence identity of 89% to SEQ ID NO:2. In embodiments, the nucleic acid sequence has a sequence identity of 90% to SEQ ID NO:2.

In embodiments, the nucleic acid sequence has a sequence identity of 91% to SEQ ID NO:2. In embodiments, the nucleic acid sequence has a sequence identity of 92% to SEQ ID NO:2. In embodiments, the nucleic acid sequence has a sequence identity of 93% to SEQ ID NO:2. In embodiments, the nucleic acid sequence has a sequence identity of 94% to SEQ ID NO:2. In embodiments, the nucleic acid sequence has a sequence identity of 95% to SEQ ID NO:2. In embodiments, the nucleic acid sequence has a sequence identity of 96% to SEQ ID NO:2. In embodiments, the nucleic acid sequence has a sequence identity of 97% to SEQ ID NO:2. In embodiments, the nucleic acid sequence has a sequence identity of 98% to SEQ ID NO:2. In embodiments, the nucleic acid sequence has a sequence identity of 99% to SEQ ID NO:2. In embodiments, the nucleic acid sequence is the sequence of SEQ ID NO:1.

In embodiments, the nucleic acid sequence has a sequence identity of about 71% to SEQ ID NO:1. In embodiments, the nucleic acid sequence has a sequence identity of about 72% to SEQ ID NO:1. In embodiments, the nucleic acid sequence has a sequence identity of about 73% to SEQ ID NO:1. In embodiments, the nucleic acid sequence has a sequence identity of about 74% to SEQ ID NO:1. In embodiments, the nucleic acid sequence has a sequence identity of about 75% to SEQ ID NO:1. In embodiments, the nucleic acid sequence has a sequence identity of about 76% to SEQ ID NO:1. In embodiments, the nucleic acid sequence has a sequence identity of about 77% to SEQ ID NO:1. In embodiments, the nucleic acid sequence has a sequence identity of about 78% to SEQ ID NO:1. In embodiments, the nucleic acid sequence has a sequence identity of about 79% to SEQ ID NO:1. In embodiments, the nucleic acid sequence has a sequence identity of about 80% to SEQ ID NO:1.

In embodiments, the nucleic acid sequence has a sequence identity of about 81% to SEQ ID NO:1. In embodiments, the nucleic acid sequence has a sequence identity of about 82% to SEQ ID NO:1. In embodiments, the nucleic acid sequence has a sequence identity of about 83% to SEQ ID NO:1. In embodiments, the nucleic acid sequence has a sequence identity of about 84% to SEQ ID NO:1. In embodiments, the nucleic acid sequence has a sequence identity of about 85% to SEQ ID NO:1. In embodiments, the nucleic acid sequence has a sequence identity of about 86% to SEQ ID NO:1. In embodiments, the nucleic acid sequence has a sequence identity of about 87% to SEQ ID NO:1. In embodiments, the nucleic acid sequence has a sequence identity of about 88% to SEQ ID NO:1. In embodiments, the nucleic acid sequence has a sequence identity of about 89% to SEQ ID NO:1. In embodiments, the nucleic acid sequence has a sequence identity of about 90% to SEQ ID NO:1.

In embodiments, the nucleic acid sequence has a sequence identity of about 91% to SEQ ID NO:1. In embodiments, the nucleic acid sequence has a sequence identity of about 92% to SEQ ID NO:1. In embodiments, the nucleic acid sequence has a sequence identity of about 93% to SEQ ID NO:1. In embodiments, the nucleic acid sequence has a sequence identity of about 94% to SEQ ID NO:1. In embodiments, the nucleic acid sequence has a sequence identity of about 95% to SEQ ID NO:1. In embodiments, the nucleic acid sequence has a sequence identity of about 96% to SEQ ID NO:1. In embodiments, the nucleic acid sequence has a sequence identity of about 97% to SEQ ID NO:1. In embodiments, the nucleic acid sequence has a sequence identity of about 98% to SEQ ID NO:1. In embodiments, the nucleic acid sequence has a sequence identity of about 99% to SEQ ID NO:1.

In embodiments, the nucleic acid sequence has a sequence identity of about 71% to SEQ ID NO:2. In embodiments, the nucleic acid sequence has a sequence identity of about 72% to SEQ ID NO:2. In embodiments, the nucleic acid sequence has a sequence identity of about 73% to SEQ ID NO:2. In embodiments, the nucleic acid sequence has a sequence identity of about 74% to SEQ ID NO:2. In embodiments, the nucleic acid sequence has a sequence identity of about 75% to SEQ ID NO:2. In embodiments, the nucleic acid sequence has a sequence identity of about 76% to SEQ ID NO:2. In embodiments, the nucleic acid sequence has a sequence identity of about 77% to SEQ ID NO:2. In embodiments, the nucleic acid sequence has a sequence identity of about 78% to SEQ ID NO:2. In embodiments, the nucleic acid sequence has a sequence identity of about 79% to SEQ ID NO:2. In embodiments, the nucleic acid sequence has a sequence identity of about 80% to SEQ ID NO:2.

In embodiments, the nucleic acid sequence has a sequence identity of about 81% to SEQ ID NO:2. In embodiments, the nucleic acid sequence has a sequence identity of about 82% to SEQ ID NO:2. In embodiments, the nucleic acid sequence has a sequence identity of about 83% to SEQ ID NO:2. In embodiments, the nucleic acid sequence has a sequence identity of about 84% to SEQ ID NO:2. In embodiments, the nucleic acid sequence has a sequence identity of about 85% to SEQ ID NO:2. In embodiments, the nucleic acid sequence has a sequence identity of about 86% to SEQ ID NO:2. In embodiments, the nucleic acid sequence has a sequence identity of about 87% to SEQ ID NO:2. In embodiments, the nucleic acid sequence has a sequence identity of about 88% to SEQ ID NO:2. In embodiments, the nucleic acid sequence has a sequence identity of about 89% to SEQ ID NO:2. In embodiments, the nucleic acid sequence has a sequence identity of about 90% to SEQ ID NO:2.

In embodiments, the nucleic acid sequence has a sequence identity of about 91% to SEQ ID NO:2. In embodiments, the nucleic acid sequence has a sequence identity of about 92% to SEQ ID NO:2. In embodiments, the nucleic acid sequence has a sequence identity of about 93% to SEQ ID NO:2. In embodiments, the nucleic acid sequence has a sequence identity of about 94% to SEQ ID NO:2. In embodiments, the nucleic acid sequence has a sequence identity of about 95% to SEQ ID NO:2. In embodiments, the nucleic acid sequence has a sequence identity of about 96% to SEQ ID NO:2. In embodiments, the nucleic acid sequence has a sequence identity of about 97% to SEQ ID NO:2. In embodiments, the nucleic acid sequence has a sequence identity of about 98% to SEQ ID NO:2. In embodiments, the nucleic acid sequence has a sequence identity of about 99% to SEQ ID NO:2.

The nucleic acid sequence may have a sequence identity of at least 70%, and the nucleic acid sequence having at least 70% sequence identity may be contiguous. In embodiments, the nucleic acid sequence has a sequence identity of at least 70%, and the nucleic acid sequence having at least 70% sequence identity is a non-contiguous sequence. A “non-contiguous sequence” as provided herein refers to a sequence including one or more sequence fragments having no sequence identity to SEQ ID NO:1 or SEQ ID NO:2. In embodiments, the non-contiguous sequence is a sequence including a first sequence fragment having at least 70% sequence identity to SEQ ID NO:1 or SEQ ID NO:2 connected to a second sequence fragment having at least 70% sequence identity to SEQ ID NO:1 or SEQ ID NO:2 through a sequence fragment having no sequence identity to SEQ ID NO:1 or SEQ ID NO:2. In embodiments, the non-contiguous sequence is a sequence including a plurality of sequence fragments having at least 70% sequence identity to SEQ ID NO:1 or SEQ ID NO:2 connected through a plurality of sequence fragments having no sequence identity to SEQ ID NO:1 or SEQ ID NO:2. In embodiments, the chimeric poxvirus further includes a nucleotide insertion, deletion or mutation.

In embodiments, the nucleic acid fragments are from cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic and vaccinia virus strain AS.

In embodiments, the chimeric poxvirus is an oncolytic virus. An oncolytic virus, as used herein is a virus capable of targeting and eliminating cancer cells. In embodiments, the oncolytic virus targets lung cancer cells. In embodiments, the oncolytic virus targets ovarian cancer cells. In embodiments, the oncolytic virus targets pancreatic cancer cells. In embodiments, the oncolytic virus preferentially targets cancer cells relative to non-cancer cells.

In embodiments, the poxvirus includes a miRNA binding sequence. In embodiments, the poxvirus includes a plurality of miRNA binding sequences. In embodiments, the plurality of miRNA binding sequences are independently different. In embodiments, the plurality of miRNA binding sequences are the same. In embodiments, the miRNA binding sequence is about 22 nucleotides in length. In embodiments, the miRNA binding sequence is at least 22 nucleotides in length. In embodiments, the miRNA binding sequence is 22 nucleotides in length. In embodiments, the miRNA binding sequence is about 22 nucleotides in length. In embodiments, each of the plurality of miRNA binding sequences is at least 22 nucleotides in length. In embodiments, each of the plurality of miRNA binding sequences is about 22 nucleotides in length. In embodiments, each of the plurality of miRNA binding sequences is 22 nucleotides in length.

In an aspect, provided is an isolated nucleic acid encoding a chimeric poxvirus as described herein. In embodiments, the isolated nucleic acid is SEQ ID NO:1. In embodiments, the isolated nucleic acid is SEQ ID NO:2.

### Chimeric Poxviruses Including Transgenes

The chimeric poxviruses as described herein may include transgenes. As used herein, a “transgene” refers to a nucleic acid sequence that originates from outside a given cell, organism or virus. A transgene as provided herein is therefore not native to, or originates within a poxvirus. A transgene as provided may encode a protein or may be a non-coding nucleic acid sequence. Transgenes provided herein may include anti-cancer nucleic acid sequences (e.g., nucleic acid binding sequences and nucleic acid sequences that encode for polypeptides useful for the treatment of cancer) or detectable moiety-encoding nucleic acid sequences. Thus, in embodiments, the chimeric poxvirus described herein includes one or more anti-cancer nucleic acid sequences or a detectable moiety-encoding nucleic acid sequence. In embodiments, the chimeric poxvirus described herein includes one or more anti-cancer nucleic acid sequences and a detectable moiety-encoding nucleic acid sequence.

The chimeric poxviruses provided herein including embodiments thereof may include transgenes. The transgenes included in the chimeric poxvirus provided herein may increase the oncolytic activity of the chimeric poxvirus compared to a chimeric poxvirus lacking said transgene. The transgenes may further increase the capability of the chimeric poxvirus to differentially express/replicate in cancer cells relative to healthy (non-cancer) cells. Where the chimeric poxvirus includes transgenes, the nucleic acid of the chimeric poxvirus includes an anti-cancer nucleic acid sequence, a nucleic acid binding sequence, a detectable moiety-encoding nucleic acid sequence or any combination thereof. Thus, in an aspect is provided a chimeric poxvirus including a nucleic acid sequence having a sequence identity of at least 70% to SEQ ID NO:1 or SEQ ID NO:2, wherein the nucleic acid sequence includes: (i) nucleic acid fragments from at least two poxvirus strains selected from the group consisting of cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, vaccinia virus strain AS, orf virus strain NZ2 and pseudocowpox virus strain TJS; (ii) one or more anti-cancer nucleic acid sequences; or (iii) a detectable moiety-encoding nucleic acid sequence.

As used herein, the terms “anti-cancer nucleic acid sequence” or “anti-cancer nucleic acid sequences” refer to nucleic acid sequences having antineoplastic properties and/or the ability to inhibit the growth or proliferation of a cancer cell and/or provide for selective expression of the chimeric poxvirus provided herein including embodiments thereof in a cancer cell relative to a healthy cell. Anti-cancer nucleic acid sequences may inhibit the progression or slow the progression of cancer temporarily or permanently. Examples of anti-cancer nucleic acid sequences include sequences encoding proteins the expression of which directly or indirectly inhibits cancer cell growth. For example, an anti-cancer nucleic acid sequence as provided herein may encode a protein, which is expressed at a higher level in a cancer cell relative to a healthy cell (e.g., sodium iodide transporter)). In another non-limiting example, an anti-cancer nucleic acid sequence may encode a polypeptide (antibody) capable of de-repressing anti-tumor immune responses (e.g., anti-PD-L1 antibodies or fragments thereof). In embodiments, the anti-cancer nucleic acid sequence includes a nucleic acid sequence capable of increasing expression/replication of a chimeric poxvirus in a cancer cell relative to a healthy cell. Thus, in embodiments, the expression (e.g., transcription, translation) rate of a chimeric poxvirus including the anti-cancer nucleic acid sequence is decreased in a healthy cell relative to a cancer cell. In embodiments, the chimeric poxvirus including the anti-cancer nucleic acid sequence is not expressed at a detectable amount in a healthy cell. In embodiments, the anti-cancer nucleic acid sequence is a nucleic acid binding sequence. In embodiments, the anti-cancer nucleic acid sequence includes a nucleic acid binding sequence.

As used herein, a “nucleic acid binding sequence” refers to a nucleic acid sequence capable of binding (hybridizing) to an at least partially complementary cellular nucleic acid (e.g., DNA, RNA, miRNA), wherein the cellular nucleic acid is present at an increased amount in a healthy cell relative to a cancer cell. The nucleic acid binding sequence provided herein may form part of the nucleic acid comprised by the chimeric poxvirus and may be operably linked to a gene of the chimeric poxvirus. Upon binding of the cellular nucleic acid to the nucleic acid binding sequence the chimeric poxvirus gene may be targeted for degradation (hydrolysis), thereby decreasing expression/replication of the chimeric poxvirus. In embodiments, the nucleic acid binding sequence is a DNA binding sequence. In embodiments, the nucleic acid binding sequence is a RNA binding sequence. In embodiments, the nucleic acid binding sequence is a miRNA binding sequence. Therefore, in embodiments, the anti-cancer nucleic acid sequence is a nucleic acid binding sequence. In embodiments, the anti-cancer nucleic acid sequence is a DNA binding sequence. In embodiments, the anti-cancer nucleic acid sequence is a RNA binding sequence. In embodiments, the anti-cancer nucleic acid sequence is a miRNA binding sequence.

A “detectable moiety-encoding nucleic acid sequence” as used herein refers to a nucleic acid sequence that encodes a composition detectable by spectroscopic, photochemical, biochemical, immunochemical, chemical, or other physical means. Detectable moiety-encoding nucleic acid sequences may encode a fluorescent moiety. Non-limiting examples of fluorescent moieties are mCherry, Emerald, and firefly luciferase.

Anti-cancer nucleic acid sequences (transgenes) may form part of the genome of the chimeric poxvirus provided herein including embodiments thereof. The chimeric poxvirus genome contains genes required for poxvirus expression and replication. Genes that are required for chimeric poxvirus expression and replication are referred to herein as “essential genes.” Genes that are not required for expression and replication of the chimeric poxvirus are referred to herein as “non-essential genes.” Anti-cancer nucleic acid sequences may be incorporated into the chimeric poxvirus genome through insertion into genes or may be operably linked to genes. Upon insertion of the anti-cancer nucleic acid sequence into a chimeric poxvirus gene, the gene (e.g., non-essential gene) or portions thereof may be deleted. In embodiments, the one or more anti-cancer nucleic acid sequences form part of a non-essential gene of the chimeric poxvirus. In embodiments, the one or more anti-cancer nucleic acid sequences are inserted into a non-essential gene of the chimeric poxvirus. In embodiments, the non-essential gene is a thymidine kinase gene. In embodiments, the non-essential gene is a J2R gene. In embodiments, the non-essential gene is a F14.5L gene.

As discussed above, anti-cancer nucleic acid sequences may encode polypeptides useful for the treatment of cancer. In embodiments, the one or more anti-cancer nucleic acid sequences independently encode a PD-L1 inhibitor or a sodium iodide symporter. In embodiments, the PD-L1 inhibitor is an anti-PD-L1 scFv. In embodiments, the anti-PD-L1 scFv includes the sequence of SEQ ID NO:17. In embodiments, the anti-PD-L1 scFv is the sequence of SEQ ID NO:17. In embodiments, the sodium iodide symporter includes the sequence of SEQ ID NO:13. In embodiments, the sodium iodide symporter is the sequence of SEQ ID NO:13.

The terms “sodium iodide symporter”, “NIS”, or “NNIS” as referred to herein include any of the recombinant or naturally-occurring forms of the sodium iodide symporter or variants or homologs thereof that maintain sodium iodide symporter activity (e.g. within at least 50%, 80%, 90%, 95%, 96%, 97%, 98%, 99% or 100% activity compared to sodium iodide symporter). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring sodium iodide symporter. In embodiments, the sodium iodide symporter is substantially identical to the protein identified by the UniProt reference number Q92911 or a variant or homolog having substantial identity thereto. In embodiments, the sodium iodide symporter includes the sequence of SEQ ID NO:13. In embodiments, the sodium iodide symporter is the sequence of SEQ ID NO:13.

Expression of the anti-cancer nucleic acid sequence provided herein may be controlled by a promoter. Therefore, in embodiments, the one or more anti-cancer nucleic acid sequences are each operably linked to a promoter. In embodiments, the promoter is a vaccinia virus early promoter. In embodiments, the promoter is a synthetic early promoter. In embodiments, the synthetic early promoter includes the sequence of SEQ ID NO:19. In embodiments, the synthetic early promoter is the sequence of SEQ ID NO:19. In embodiments, the promoter is a vaccinia virus late promoter. In embodiments, the promoter is a H5 promoter or an 11K promoter. In embodiments, the H5 promoter includes the sequence of SEQ ID NO:18. In embodiments, the H5 promoter is the sequence of SEQ ID NO:18. In embodiments, the 11K promoter includes the sequence of SEQ ID NO:20. In embodiments, the 11K promoter is the sequence of SEQ ID NO:20.

The anti-cancer nucleic acid sequence (nucleic acid binding sequence) provided herein may be incorporated into the chimeric poxvirus genome such that it is placed into functional relationship (operably linked) with a specific poxvirus gene. For example, the anti-cancer nucleic acid sequence (nucleic acid binding sequence) may be operably linked to a poxvirus gene if it affects the transcription or translation of the poxvirus gene. Generally, the anti-cancer nucleic acid sequence (nucleic acid binding sequence) and the poxvirus gene are operably linked when they are contiguous and/or in reading phase. In embodiments, the one or more anti-cancer nucleic acid sequences (one or more nucleic acid binding sequences) are operably linked to an essential gene of the chimeric poxvirus. In embodiments, the one or more anti-cancer nucleic acid sequences (one or more nucleic acid binding sequences) are operably linked to a DNA polymerase gene of the chimeric poxvirus. In embodiments, the one or more anti-cancer nucleic acid sequences (one or more nucleic acid binding sequences) are operably linked to the 3′ end of a DNA polymerase gene of the chimeric poxvirus. In embodiments, the one or more anti-cancer nucleic acid sequences (one or more nucleic acid binding sequences) are operably linked to a uracil DNA glycosylase gene. In embodiments, the one or more anti-cancer nucleic acid sequences (one or more nucleic acid binding sequences) are operably linked to the 3′end of a uracil DNA glycosylase gene.

In embodiments, the one or more anti-cancer nucleic acid sequences (one or more nucleic acid binding sequences) independently encode for a miRNA binding sequence. In embodiments, the miRNA binding sequence is a miR100 binding sequence or a let7c binding sequence. In embodiments, the miRNA binding sequence is a miR100 binding sequence. In embodiments, the miR100 binding sequence includes the sequence of SEQ ID NO:9. In embodiments, the miR100 binding sequence is the sequence of SEQ ID NO:9. In embodiments, the miR100 binding sequence includes the sequence of SEQ ID NO:10. In embodiments, the miR100 binding sequence is the sequence of SEQ ID NO:10. In embodiments, the miRNA binding sequence is a let7c binding sequence. In embodiments, the let7c binding sequence includes the sequence of SEQ ID NO:11. In embodiments, the let7c binding sequence is the sequence of SEQ ID NO:11.

In embodiments, the one or more anti-cancer nucleic acid sequences are a first anti-cancer nucleic acid sequence and a second anti-cancer nucleic acid sequence. As provided herein the first anti-cancer nucleic acid sequence may be a first nucleic acid binding sequence and the second anti-cancer nucleic acid sequence may be a second nucleic acid binding sequence.

In embodiments, the first anti-cancer nucleic acid sequence encodes a sodium iodide symporter and said second anti-cancer nucleic acid sequence (second nucleic acid binding sequence) encodes a miRNA binding sequence. In embodiments, the first anti-cancer nucleic acid sequence forms part of a thymidine kinase gene and the second anti-cancer nucleic acid sequence (second nucleic acid binding sequence) is operably linked to a uracil DNA glycosylase gene. In embodiments, the first anti-cancer nucleic acid sequence forms part of a thymidine kinase gene and the second anti-cancer nucleic acid sequence (second nucleic acid binding sequence) is operably linked to a DNA polymerase gene.

In embodiments, the first anti-cancer nucleic acid sequence encodes a sodium iodide symporter and the second anti-cancer nucleic acid sequence encodes a PD-L1 inhibitor. In embodiments, the first anti-cancer nucleic acid sequence forms part of a thymidine kinase gene and the second anti-cancer nucleic acid sequence forms part of a F14.5L gene.

In embodiments, the nucleic acid sequence includes: (i) nucleic acid fragments from at least two poxvirus strains selected from the group consisting of cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, vaccinia virus strain AS, orf virus strain NZ2 and pseudocowpox virus strain TJS; and (ii) said detectable moiety-encoding nucleic acid sequence. In embodiments, the detectable moiety-encoding nucleic acid sequence encodes a fluorescent moiety. In embodiments, the detectable moiety-encoding nucleic acid sequence forms part of a non-essential gene of the chimeric poxvirus. In embodiments, the non-essential gene is a thymidine kinase gene. In embodiments, the parts of the non-essential gene are deleted.

In embodiments, the detectable moiety-encoding nucleic acid sequence is operably linked to a promoter. In embodiments, the promoter is a vaccinia virus early promoter. In embodiments, the promoter is a synthetic early promoter. In embodiments, the synthetic early promoter includes the sequence of SEQ ID NO:19. In embodiments, the synthetic early promoter is the sequence of SEQ ID NO:19. In embodiments, the promoter is a vaccinia virus late promoter. In embodiments, the promoter is a H5 promoter or an 11K promoter. In embodiments, the H5 promoter includes the sequence of SEQ ID NO:18. In embodiments, the H5 promoter is the sequence of SEQ ID NO:18. In embodiments, the 11K promoter includes the sequence of SEQ ID NO:20. In embodiments, the 11K promoter is the sequence of SEQ ID NO:20.

In one embodiment, the anti-cancer nucleic acid sequence (nucleic acid binding sequence) encodes a miRNA binding sequence having the sequence of SEQ ID NO:10 and is operably linked to the 3′ end of a uracil DNA glycosylase gene.

In one embodiment, the anti-cancer nucleic acid sequence (nucleic acid binding sequence) encodes a miRNA binding sequence having the sequence of SEQ ID NO:9 and is operably linked to the 3′ end of a uracil DNA glycosylase gene.

In one embodiment, the anti-cancer nucleic acid sequence (nucleic acid binding sequence) encodes a miRNA binding sequence having the sequence of SEQ ID NO:11 and is operably linked to the 3′ end of a uracil DNA glycosylase gene.

In one embodiment, the anti-cancer nucleic acid sequence (nucleic acid binding sequence) encodes a miRNA binding sequence having the sequence of SEQ ID NO:9 and is operably linked to the 3′ end of a DNA polymerase gene.

In one embodiment, the anti-cancer nucleic acid sequence (nucleic acid binding sequence) encodes a miRNA binding sequence having the sequence of SEQ ID NO:11 and is operably linked to the 3′ end of a DNA polymerase gene.

In one embodiment, the thymidine kinase gene of the chimeric poxvirus has the sequence of SEQ ID NO:5.

In one embodiment, the anti-cancer nucleic acid sequence encodes a sodium iodide symporter, is operably linked to a synthetic early promoter, and forms part of a thymidine kinase gene, wherein the sodium iodide symporter has the sequence of SEQ ID NO:13 and the synthetic early promoter has the sequence of SEQ ID NO:19.

In one embodiment, the detectable moiety-encoding nucleic acid sequence encodes a fluorescent moiety having the sequence of SEQ ID NO:14, is operably linked to a H5 promoter, and forms part of a thymidine kinase gene, wherein the H5 promoter has the sequence of SEQ ID NO:18.

In one embodiment, the detectable moiety-encoding nucleic acid sequence encodes a fluorescent moiety having the sequence of SEQ ID NO:15, is operably linked to a synthetic early promoter, and forms part of a thymidine kinase gene, wherein the synthetic early promoter has the sequence of SEQ ID NO:19.

In one embodiment, the detectable moiety-encoding nucleic acid sequence encodes a fluorescent moiety having the sequence of SEQ ID NO:15, is operably linked to a H5 promoter, and forms part of a thymidine kinase gene, wherein the H5 promoter has the sequence of SEQ ID NO:18.

In one embodiment, the detectable moiety-encoding nucleic acid sequence encodes a fluorescent moiety having the sequence of SEQ ID NO:15, is operably linked to an 11K promoter, and forms part of a thymidine kinase gene, wherein the 11K promoter has the sequence of SEQ ID NO:20.

In one embodiment, the detectable moiety-encoding nucleic acid sequence encodes a fluorescent moiety having the sequence of SEQ ID NO:16, is operably linked to a H5 promoter, and forms part of a thymidine kinase gene, wherein the H5 promoter has the sequence of SEQ ID NO:18.

In one embodiment, the detectable moiety-encoding nucleic acid sequence encodes a fluorescent moiety having the sequence of SEQ ID NO:16, is operably linked to an 11K promoter, and forms part of a thymidine kinase gene, wherein the 11K promoter has the sequence of SEQ ID NO:20.

In one embodiment, the first anti-cancer nucleic acid sequence encodes a sodium iodide symporter, is operably linked to a synthetic early promoter, and forms part of a thymidine kinase gene; and the second anti-cancer nucleic acid sequence (nucleic acid binding sequence) encodes a miRNA binding sequence having the sequence of SEQ ID NO:10, and is operably linked to the 3′ end of a uracil DNA glycosylase gene, wherein the sodium iodide symporter has the sequence of SEQ ID NO:13 and the synthetic early promoter has the sequence of SEQ ID NO:19.

In one embodiment, the first anti-cancer nucleic acid sequence encodes a sodium iodide symporter, is operably linked to a synthetic early promoter, and forms part of a thymidine kinase gene; and the second anti-cancer nucleic acid sequence (nucleic acid binding sequence) encodes a miRNA binding sequence having the sequence of SEQ ID NO:9, and is operably linked to the 3′ end of a uracil DNA glycosylase gene, wherein the sodium iodide symporter has the sequence of SEQ ID NO:13 and the synthetic early promoter has the sequence of SEQ ID NO:19.

In one embodiment, the first anti-cancer nucleic acid sequence encodes a sodium iodide symporter, is operably linked to a synthetic early promoter, and forms part of a thymidine kinase gene; and the second anti-cancer nucleic acid sequence (nucleic acid binding sequence) encodes a miRNA binding sequence having the sequence of SEQ ID NO:11, and is operably linked to the 3′ end of a uracil DNA glycosylase gene, wherein the sodium iodide symporter has the sequence of SEQ ID NO:13 and the synthetic early promoter has the sequence of SEQ ID NO:19.

In one embodiment, the first anti-cancer nucleic acid sequence encodes a sodium iodide symporter, is operably linked to a synthetic early promoter, and forms part of a thymidine kinase gene; and the second anti-cancer nucleic acid sequence (nucleic acid binding sequence) encodes a miRNA binding sequence having the sequence of SEQ ID NO:9, and is operably linked to the 3′ end of a DNA polymerase gene, wherein the sodium iodide symporter has the sequence of SEQ ID NO:13 and the synthetic early promoter has the sequence of SEQ ID NO:19.

In one embodiment, the first anti-cancer nucleic acid sequence encodes a sodium iodide symporter, is operably linked to a synthetic early promoter, and forms part of a thymidine kinase gene; and the second anti-cancer nucleic acid sequence (nucleic acid binding sequence) encodes a miRNA binding sequence having the sequence of SEQ ID NO:11, and is operably linked to the 3′ end of a DNA polymerase gene, wherein the sodium iodide symporter has the sequence of SEQ ID NO:13 and the synthetic early promoter has the sequence of SEQ ID NO:19.

In one embodiment, the F14.5L gene of the chimeric poxvirus has the sequence of SEQ ID NO:7.

In one embodiment, the anti-cancer nucleic acid sequence encodes an anti-PD-L1 scFv, is operably linked to a H5 promoter, and forms part of a F14.5L gene, wherein the anti-PD-L1 scFv has the sequence of SEQ ID NO:17 and the H5 promoter has the sequence of SEQ ID NO:18.

In one embodiment, the anti-cancer nucleic acid sequence encodes a sodium iodide symporter, is operably linked to a synthetic early promoter, and forms part of a thymidine kinase gene, and the F14.5L gene has the sequence of SEQ ID NO:7, wherein the sodium iodide symporter has the sequence of SEQ ID NO:13 and the synthetic early promoter has the sequence of SEQ ID NO:19.

In one embodiment, the first anti-cancer nucleic acid sequence encodes a sodium iodide symporter, is operably linked to a synthetic early promoter, and forms part of a thymidine kinase gene; and the second anti-cancer nucleic acid sequence encodes an anti-PD-L1 scFv, is operably linked to a H5 promoter and forms part of a F14.5L gene, wherein the sodium iodide symporter has the sequence of SEQ ID NO:13, the synthetic early promoter has the sequence of SEQ ID NO:19, the anti-PD-L1 scFv has the sequence of SEQ ID NO:17, and the H5 promoter has the sequence of SEQ ID NO:18.

In an aspect is provided a composition including a chimeric poxvirus and a neural stem cell (NSC). In embodiments, the chimeric poxvirus forms part of the NSC. Where the chimeric poxvirus forms part of the NSC, the NSC includes the chimeric poxvirus (the genome of the chimeric poxvirus, viral particles of the chimeric poxvirus or a combination thereof). In embodiments, the poxvirus is encoded by a nucleic acid sequence having a sequence identity of at least 70% to SEQ ID NO:1. In embodiments, the nucleic acid sequence includes nucleic acid fragments from at least two poxvirus strains selected from the group consisting of cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, vaccinia virus strain AS, orf virus strain NZ2 and pseudocowpox virus strain TJS. In embodiments, the nucleic acid sequence includes nucleic acid fragments from cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic and vaccinia virus strain AS.

In embodiments, the nucleic acid sequence further includes one or more anti-cancer nucleic acid sequences or a detectable moiety-encoding nucleic acid sequence. In embodiments, the chimeric poxvirus is a replication-competent chimeric poxvirus. In embodiments, the NSC is a Human Leukocyte Antigen (HLA) II-negative NSC. In embodiments, the NSC is an HB1.F3.CD21 cell.

In embodiments, the composition further includes an effective amount of a therapeutic agent. In embodiments, the therapeutic agent is an anti-cancer agent. In embodiments, the therapeutic agent is a PD-L1 inhibitor, a CTLA-4 inhibitor or an OX40 inhibitor. In embodiments, the therapeutic agent is a PD-L1 inhibitor. In embodiments, the therapeutic agent is a CTLA-4 inhibitor. In embodiments, the therapeutic agent is an OX40 inhibitor. In embodiments, the therapeutic agent is an anti-PD-L1 antibody, an anti-CTLA-4 antibody or an anti-OX40 antibody. In embodiments, the therapeutic agent is an anti-PD-L1 antibody. In embodiments, the therapeutic agent is an anti-CTLA-4 antibody. In embodiments, the therapeutic agent is an anti-OX40 antibody.

In embodiments, the composition is effective to treat cancer. In embodiments, the cancer is breast cancer, colon cancer, kidney cancer, leukemia, lung cancer, melanoma, ovarian cancer, prostate cancer, pancreatic cancer, brain cancer, liver cancer, gastric cancer or a sarcoma. In embodiments, the cancer is ovarian cancer. In embodiments, the ovarian cancer is an ovarian metastasis.

### III. Pharmaceutical Compositions

The neural stem cell including the chimeric poxvirus provided herein, including embodiments thereof, may form part of a pharmaceutical composition. Thus, in embodiments, the composition is a pharmaceutical composition. In embodiments, the pharmaceutical composition includes a pharmaceutically acceptable excipient.

The term “preparation” is intended to include the formulation of the active compound (e.g., neural stem cell including a chimeric poxvirus) with encapsulating material as a carrier providing a capsule in which the active component with or without other carriers, is surrounded by a carrier, which is thus in association with it. Similarly, cachets and lozenges are included.

Tablets, powders, capsules, pills, cachets, and lozenges can be used as solid dosage forms suitable for oral administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The compositions provided herein including embodiments thereof may be adminstered orally, gastrointestinally, or rectally. Administration can be in the form of a single bolus dose, or may be, for example, by a continuous perfusion pump. In embodiments, the chimeric poxvirus provided herein is combined with one or more excipients, for example, a disintegrant, a filler, a glidant, or a preservative. In embodiments, the chimeric poxvirus provided herein forms part of a capsule. Suitable capsules include both hard shell capsules or soft-shelled capsules. Any lipid-based or polymer-based colloid may be used to form the capusule. Exemplary polymers useful for colloid preparations include gelatin, plant polysaccharides or their derivatives such as carrageenans and modified forms of starch and cellulose, e.g., hypromellose. Optionally, other ingredients may be added to the gelling agent solution, for example plasticizers such as glycerin and/or sorbitol to decrease the capsule's hardness, coloring agents, preservatives, disintegrants, lubricants and surface treatment.

The compositions can be formulated in a unit dosage form, each dosage containing, for example, from about 0.1-10×10⁶ neural stem cells including chimeric poxvirus. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules.

In some embodiments, tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

### IV. Cell Compositions

In another aspect, is provided a neural stem cell (NSC) including a chimeric poxvirus. In embodiments, the poxvirus is encoded by a nucleic acid sequence having a sequence identity of at least 70% to SEQ ID NO:1. In embodiments, the nucleic acid sequence including nucleic acid fragments from at least two poxvirus strains selected from the group consisting of cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, vaccinia virus strain AS, orf virus strain NZ2 and pseudocowpox virus strain TJS. In embodiments, the nucleic acid sequence including nucleic acid fragments from cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic and vaccinia virus strain AS.

In embodiments, the nucleic acid sequence further including one or more anti-cancer nucleic acid sequences or a detectable moiety-encoding nucleic acid sequence. In embodiments, the chimeric poxvirus is a replication-competent chimeric poxvirus. In embodiments, the NSC is a Human Leukocyte Antigen (HLA) II-negative NSC. In embodiments, the NSC is an HB1.F3.CD21 cell. In embodiments, the NSC forms part of a pharmaceutical composition. In embodiments, the pharmaceutical composition including a pharmaceutically acceptable excipient.

### V. Methods of Treatment

In an another aspect is provided a method of treating cancer in a subject in need thereof, the method including administering to the subject a therapeutically effective amount of a neural stem cell and a chimeric poxvirus as described herein including embodiments thereof, thereby treating cancer in the subject. In embodiments, the cancer is breast cancer, colon cancer, kidney cancer, leukemia, lung cancer, melanoma, ovarian cancer, prostate cancer, pancreatic cancer, brain cancer, liver cancer, gastric cancer or a sarcoma. In embodiments, the cancer is breast cancer. In embodiments, the cancer is colon cancer. In embodiments, the cancer is kidney cancer. In embodiments, the cancer is leukemia. In embodiments, the cancer is lung cancer. In embodiments, the cancer is melanoma. In embodiments, the cancer is ovarian cancer. In embodiments, the cancer is prostate cancer. In embodiments, the cancer is pancreatic cancer. In embodiments, the cancer is brain cancer. In embodiments, the cancer is liver cancer. In embodiments, the cancer is gastric cancer. In embodiments, the cancer is a sarcoma. In embodiments, the cancer is triple-negative breast cancer.

In embodiments, the administering includes administering a neural stem cell including a chimeric poxvirus and a therapeutic agent. In embodiments, the neural stem cell including a chimeric poxvirus and the therapeutic agent are administered at a combined synergistic amount. In embodiments, the neural stem cell including a chimeric poxvirus and the therapeutic agent are administered simultaneously. In embodiments, the neural stem cell including a chimeric poxvirus and the therapeutic agent are administered sequentially.

In embodiments, the neural stem cell is infected with at least 10³ plaque forming units (Pfu)/kg of chimeric poxvirus prior to the administering step. In embodiments, the neural stem cell is infected with at least 10⁴ plaque forming units (Pfu)/kg of chimeric poxvirus prior to the administering step. In embodiments, the neural stem cell is infected with at least 10⁵ plaque forming units (Pfu)/kg of chimeric poxvirus prior to the administering step. In embodiments, the neural stem cell is infected with at least 10⁶ plaque forming units (Pfu)/kg of chimeric poxvirus prior to the administering step. In embodiments, the neural stem cell is infected with at least 10⁷ plaque forming units (Pfu)/kg of chimeric poxvirus prior to the administering step. In embodiments, the neural stem cell is infected with at least 10⁸ plaque forming units (Pfu)/kg of chimeric poxvirus prior to the administering step.

In embodiments, the neural stem cell is infected with 10³ plaque forming units (Pfu)/kg of chimeric poxvirus prior to the administering step. In embodiments, the neural stem cell is infected with 10⁴ plaque forming units (Pfu)/kg of chimeric poxvirus prior to the administering step. In embodiments, the neural stem cell is infected with 10⁵ plaque forming units (Pfu)/kg of chimeric poxvirus prior to the administering step. In embodiments, the neural stem cell is infected with 10⁶ plaque forming units (Pfu)/kg of chimeric poxvirus prior to the administering step. In embodiments, the neural stem cell is infected with 10⁷ plaque forming units (Pfu)/kg of chimeric poxvirus prior to the administering step. In embodiments, the neural stem cell is infected with 10⁸ plaque forming units (Pfu)/kg of chimeric poxvirus prior to the administering step.

In embodiments, the neural stem cell is infected with about 10³ plaque forming units (Pfu)/kg of chimeric poxvirus prior to the administering step. In embodiments, the neural stem cell is infected with 10³ plaque forming units (Pfu)/kg of chimeric poxvirus prior to the administering step. In embodiments, the neural stem cell is infected with about 4×10⁴ plaque forming units (Pfu)/kg of chimeric poxvirus prior to the administering step. In embodiments, the neural stem cell is infected with 4×10⁴ plaque forming units (Pfu)/kg of chimeric poxvirus prior to the administering step. In embodiments, the neural stem cell is infected with about 5×10⁴ plaque forming units (Pfu)/kg of chimeric poxvirus prior to the administering step. In embodiments, the neural stem cell is infected with 5×10⁴ plaque forming units (Pfu)/kg of chimeric poxvirus prior to the administering step.

In embodiments, the neural stem cell is infected with the chimeric poxvirus prior to the administering step. In embodiments, the neural stem cell is infected with the chimeric poxvirus at a multiplicity of infection of 0.5. In embodiments, the neural stem cell is infected with the chimeric poxvirus at a multiplicity of infection of 1. In embodiments, the neural stem cell is infected with the chimeric poxvirus at a multiplicity of infection of 1.5. In embodiments, the neural stem cell is infected with the chimeric poxvirus at a multiplicity of infection of 2. In embodiments, the neural stem cell is infected with the chimeric poxvirus at a multiplicity of infection of 2.5. In embodiments, the neural stem cell is infected with the chimeric poxvirus at a multiplicity of infection of 3. In embodiments, the neural stem cell is infected with the chimeric poxvirus at a multiplicity of infection of 3.5. In embodiments, the neural stem cell is infected with the chimeric poxvirus at a multiplicity of infection of 4. In embodiments, the neural stem cell is infected with the chimeric poxvirus at a multiplicity of infection of 4.5. In embodiments, the neural stem cell is infected with the chimeric poxvirus at a multiplicity of infection of 5. In embodiments, the neural stem cell is infected with the chimeric poxvirus at a multiplicity of infection of 5.5. In embodiments, the neural stem cell is infected with the chimeric poxvirus at a multiplicity of infection of 6. Thus, the chimerci poxvirus forms part of the NSC prior to the administering step. Wherein the chimeric poxvirus forms part of the NSC, the NSC includes the chimeric poxvirus. Therefore, unless otherwise stated, where the NSC provided herein is administered it includes the chimeric poxvirus as provided herein including embodiments thereof.

In embodiments, the neural stem cell including the chimeric poxvirus is administered at a cell number of 1×10⁷ cells. In embodiments, the neural stem cell including the chimeric poxvirus is administered at a cell number of 2×10⁷ cells. In embodiments, the neural stem cell including the chimeric poxvirus is administered at a cell number of 3×10⁷ cells. In embodiments, the neural stem cell including the chimeric poxvirus is administered at a cell number of 4×10⁷ cells. In embodiments, the neural stem cell including the chimeric poxvirus is administered at a cell number of 5×10⁷ cells. In embodiments, the neural stem cell including the chimeric poxvirus is administered at a cell number of 6×10⁷ cells. In embodiments, the neural stem cell including the chimeric poxvirus is administered at a cell number of 7×10⁷ cells. In embodiments, the neural stem cell including the chimeric poxvirus is administered at a cell number of 8×10⁷ cells. In embodiments, the neural stem cell including the chimeric poxvirus is administered at a cell number of 9×10⁷ cells. In embodiments, the neural stem cell including the chimeric poxvirus is administered at a cell number of 1×10⁸ cells. In embodiments, the neural stem cell including the chimeric poxvirus is administered at a cell number of 1.5×10⁸ cells. In embodiments, the neural stem cell including the chimeric poxvirus is administered at a cell number of 1.8×10⁸ cells. In embodiments, the neural stem cell including the chimeric poxvirus is administered at a cell number of 2×10⁸ cells. In embodiments, the neural stem cell including the chimeric poxvirus is administered at a cell number of 2.5×10⁸ cells. In embodiments, the neural stem cell including the chimeric poxvirus is administered at a cell number of 3×10⁸ cells. In embodiments, the neural stem cell including the chimeric poxvirus is administered at a cell number of 3.5×10⁸ cells. In embodiments, the neural stem cell including the chimeric poxvirus is administered at a cell number of 4×10⁸ cells. In embodiments, the neural stem cell including the chimeric poxvirus is administered at a cell number of 4.5×10⁸ cells. In embodiments, the neural stem cell including the chimeric poxvirus is administered at a cell number of 5×10⁸ cells.

In embodiments, the neural stem cell is administered at a cell number of 1×10⁷ cells. In embodiments, the neural stem cell is administered at a cell number of 2×10⁷ cells. In embodiments, the neural stem cell is administered at a cell number of 3×10⁷ cells. In embodiments, the neural stem cell is administered at a cell number of 4×10⁷ cells. In embodiments, the neural stem cell is administered at a cell number of 5×10⁷ cells. In embodiments, the neural stem cell is administered at a cell number of 6×10⁷ cells. In embodiments, the neural stem cell is administered at a cell number of 7×10⁷ cells. In embodiments, the neural stem cell is administered at a cell number of 8×10⁷ cells. In embodiments, the neural stem cell is administered at a cell number of 9×10⁷ cells. In embodiments, the neural stem cell is administered at a cell number of 1×10⁸ cells. In embodiments, the neural stem cell is administered at a cell number of 1.5×10⁸ cells. In embodiments, the neural stem cell is administered at a cell number of 1.8×10⁸ cells. In embodiments, the neural stem cell is administered at a cell number of 2×10⁸ cells. In embodiments, the neural stem cell is administered at a cell number of 2.5×10⁸ cells. In embodiments, the neural stem cell is administered at a cell number of 3×10⁸ cells. In embodiments, the neural stem cell is administered at a cell number of 3.5×10⁸ cells. In embodiments, the neural stem cell is administered at a cell number of 4×10⁸ cells. In embodiments, the neural stem cell is administered at a cell number of 4.5×10⁸ cells. In embodiments, the neural stem cell is administered at a cell number of 5×10⁸ cells.

In embodiments, the neural stem cell including the chimeric poxvirus is administered at a cell number of 1×10⁷ to 1×10⁸ cells. In embodiments, the neural stem cell including the chimeric poxvirus is administered at a cell number of 1.5×10⁷ to 1×10⁸ cells. In embodiments, the neural stem cell including the chimeric poxvirus is administered at a cell number of 2×10⁷ to 1×10⁸ cells. In embodiments, the neural stem cell including the chimeric poxvirus is administered at a cell number of 2.5×10⁷ to 1×10⁸ cells. In embodiments, the neural stem cell including the chimeric poxvirus is administered at a cell number of 3×10⁷ to 1×10⁸ cells. In embodiments, the neural stem cell including the chimeric poxvirus is administered at a cell number of 3.5×10⁷ to 1×10⁸ cells. In embodiments, the neural stem cell including the chimeric poxvirus is administered at a cell number of 4×10⁷ to 1×10⁸ cells. In embodiments, the neural stem cell including the chimeric poxvirus is administered at a cell number of 4.5×10⁷ to 1×10⁸ cells. In embodiments, the neural stem cell including the chimeric poxvirus is administered at a cell number of 5×10⁷ to 1×10⁸ cells. In embodiments, the neural stem cell including the chimeric poxvirus is administered at a cell number of 5.5×10⁷ to 1×10⁸ cells. In embodiments, the neural stem cell including the chimeric poxvirus is administered at a cell number of 6×10⁷ to 1×10⁸ cells. In embodiments, the neural stem cell including the chimeric poxvirus is administered at a cell number of 6.5×10⁷ to 1×10⁸ cells. In embodiments, the neural stem cell including the chimeric poxvirus is administered at a cell number of 7×10⁷ to 1×10⁸ cells. In embodiments, the neural stem cell including the chimeric poxvirus is administered at a cell number of 7.5×10⁷ to 1×10⁸ cells. In embodiments, the neural stem cell including the chimeric poxvirus is administered at a cell number of 8×10⁷ to 1×10⁸ cells. In embodiments, the neural stem cell including the chimeric poxvirus is administered at a cell number of 8.5×10⁷ to 1×10⁸ cells. In embodiments, the neural stem cell including the chimeric poxvirus is administered at a cell number of 9×10⁷ to 1×10⁸ cells. In embodiments, the neural stem cell including the chimeric poxvirus is administered at a cell number of 9.5×10⁷ to 1×10⁸ cells.

In embodiments, the neural stem cell is administered at a cell number of 1×10⁷ to 1×10⁸ cells. In embodiments, the neural stem cell is administered at a cell number of 1.5×10⁷ to 1×10⁸ cells. In embodiments, the neural stem cell is administered at a cell number of 2×10⁷ to 1×10⁸ cells. In embodiments, the neural stem cell is administered at a cell number of 2.5×10⁷ to 1×10⁸ cells. In embodiments, the neural stem cell is administered at a cell number of 3×10⁷ to 1×10⁸ cells. In embodiments, the neural stem cell is administered at a cell number of 3.5×10⁷ to 1×10⁸ cells. In embodiments, the neural stem cell is administered at a cell number of 4×10⁷ to 1×10⁸ cells. In embodiments, the neural stem cell is administered at a cell number of 4.5×10⁷ to 1×10⁸ cells. In embodiments, the neural stem cell is administered at a cell number of 5×10⁷ to 1×10⁸ cells. In embodiments, the neural stem cell is administered at a cell number of 5.5×10⁷ to 1×10⁸ cells. In embodiments, the neural stem cell is administered at a cell number of 6×10⁷ to 1×10⁸ cells. In embodiments, the neural stem cell is administered at a cell number of 6.5×10⁷ to 1×10⁸ cells. In embodiments, the neural stem cell is administered at a cell number of 7×10⁷ to 1×10⁸ cells. In embodiments, the neural stem cell is administered at a cell number of 7.5×10⁷ to 1×10⁸ cells. In embodiments, the neural stem cell is administered at a cell number of 8×10⁷ to 1×10⁸ cells. In embodiments, the neural stem cell is administered at a cell number of 8.5×10⁷ to 1×10⁸ cells. In embodiments, the neural stem cell is administered at a cell number of 9×10⁷ to 1×10⁸ cells. In embodiments, the neural stem cell is administered at a cell number of 9.5×10⁷ to 1×10⁸ cells.

In embodiments, the neural stem cell including the chimeric poxvirus is administered at a cell number of 5.6×10⁶ cells/ml. In embodiments, the neural stem cell including the chimeric poxvirus is administered at a cell number of 1.1×10⁷ cells/ml. In embodiments, the neural stem cell including the chimeric poxvirus is administered at a cell number of 1.7×10⁷ cells/ml. In embodiments, the neural stem cell including the chimeric poxvirus is administered at a cell number of 3.3×10⁷ cells/ml. In embodiments, the neural stem cell including the chimeric poxvirus is administered at a cell number of 5×10⁷ cells/ml. In embodiments, the neural stem cell including the chimeric poxvirus is administered at a cell number of 5×10⁶ cells/ml. In embodiments, the neural stem cell including the chimeric poxvirus is administered at a cell number of 6×10⁶ cells/ml. In embodiments, the neural stem cell including the chimeric poxvirus is administered at a cell number of 7×10⁶ cells/ml. In embodiments, the neural stem cell including the chimeric poxvirus is administered at a cell number of 8×10⁶ cells/ml. In embodiments, the neural stem cell including the chimeric poxvirus is administered at a cell number of 9×10⁶ cells/ml. In embodiments, the neural stem cell including the chimeric poxvirus is administered at a cell number of 1×10⁷ cells/ml. In embodiments, the neural stem cell including the chimeric poxvirus is administered at a cell number of 2×10⁷ cells/ml. In embodiments, the neural stem cell including the chimeric poxvirus is administered at a cell number of 3×10⁷ cells/ml. In embodiments, the neural stem cell including the chimeric poxvirus is administered at a cell number of 4×10⁷ cells/ml. In embodiments, the neural stem cell including the chimeric poxvirus is administered at a cell number of 5×10⁷ cells/ml. In embodiments, the neural stem cell including the chimeric poxvirus is administered at a cell number of 6×10⁷ cells/ml. In embodiments, the neural stem cell including the chimeric poxvirus is administered at a cell number of 7×10⁷ cells/ml. In embodiments, the neural stem cell including the chimeric poxvirus is administered at a cell number of 8×10⁷ cells/ml. In embodiments, the neural stem cell including the chimeric poxvirus is administered at a cell number of 9×10⁷ cells/ml. In embodiments, the neural stem cell including the chimeric poxvirus is administered at a cell number of 1×10⁸ cells/ml.

In embodiments, the neural stem cell including the chimeric poxvirus is administered at a cell number of 1×10⁶ to 1×10⁸ cells/ml. In embodiments, the neural stem cell including the chimeric poxvirus is administered at a cell number of 2×10⁶ to 1×10⁸ cells/ml. In embodiments, the neural stem cell including the chimeric poxvirus is administered at a cell number of 3×10⁶ to 1×10⁸ cells/ml. In embodiments, the neural stem cell including the chimeric poxvirus is administered at a cell number of 4×10⁶ to 1×10⁸ cells/ml. In embodiments, the neural stem cell including the chimeric poxvirus is administered at a cell number of 5×10⁶ to 1×10⁸ cells/ml. In embodiments, the neural stem cell including the chimeric poxvirus is administered at a cell number of 6×10⁶ to 1×10⁸ cells/ml. In embodiments, the neural stem cell including the chimeric poxvirus is administered at a cell number of 7×10⁶ to 1×10⁸ cells/ml. In embodiments, the neural stem cell including the chimeric poxvirus is administered at a cell number of 8×10⁶ to 1×10⁸ cells/ml. In embodiments, the neural stem cell including the chimeric poxvirus is administered at a cell number of 9×10⁶ to 1×10⁸ cells/ml. In embodiments, the neural stem cell including the chimeric poxvirus is administered at a cell number of 1×10⁷ to 1×10⁸ cells/ml.

In embodiments, the neural stem cell including the chimeric poxvirus is administered at a cell number of 2×10⁷ to 1×10⁸ cells/ml. In embodiments, the neural stem cell including the chimeric poxvirus is administered at a cell number of 3×10⁷ to 1×10⁸ cells/ml. In embodiments, the neural stem cell including the chimeric poxvirus is administered at a cell number of 4×10⁷ to 1×10⁸ cells/ml. In embodiments, the neural stem cell including the chimeric poxvirus is administered at a cell number of 5×10⁷ to 1×10⁸ cells/ml. In embodiments, the neural stem cell including the chimeric poxvirus is administered at a cell number of 6×10⁷ to 1×10⁸ cells/ml. In embodiments, the neural stem cell including the chimeric poxvirus is administered at a cell number of 7×10⁷ to 1×10⁸ cells/ml. In embodiments, the neural stem cell including the chimeric poxvirus is administered at a cell number of 8×10⁷ to 1×10⁸ cells/ml. In embodiments, the neural stem cell including the chimeric poxvirus is administered at a cell number of 9×10⁷ to 1×10⁸ cells/ml.

In embodiments, the neural stem cell including the chimeric poxvirus is administered at a cell number of 0.1×10⁶ to 20×10⁶ cells. In embodiments, the neural stem cell including the chimeric poxvirus is administered at a cell number of 0.1×10⁶. In embodiments, the neural stem cell including the chimeric poxvirus is administered at a cell number of 0.5×10⁶. In embodiments, the neural stem cell including the chimeric poxvirus is administered at a cell number of 1×10⁶. In embodiments, the neural stem cell including the chimeric poxvirus is administered at a cell number of 2×10⁶. In embodiments, the neural stem cell including the chimeric poxvirus is administered at a cell number of 3×10⁶. In embodiments, the neural stem cell including the chimeric poxvirus is administered at a cell number of 4×10⁶. In embodiments, the neural stem cell including the chimeric poxvirus is administered at a cell number of 5×10⁶. In embodiments, the neural stem cell including the chimeric poxvirus is administered at a cell number of 6×10⁶. In embodiments, the neural stem cell including the chimeric poxvirus is administered at a cell number of 7×10⁶. In embodiments, the neural stem cell including the chimeric poxvirus is administered at a cell number of 8×10⁶. In embodiments, the neural stem cell including the chimeric poxvirus is administered at a cell number of 9×10⁶. In embodiments, the neural stem cell including the chimeric poxvirus is administered at a cell number of 10×10⁶. In embodiments, the neural stem cell including the chimeric poxvirus is administered at a cell number of 11×10⁶. In embodiments, the neural stem cell including the chimeric poxvirus is administered at a cell number of 12×10⁶. In embodiments, the neural stem cell including the chimeric poxvirus is administered at a cell number of 13×10⁶. In embodiments, the neural stem cell including the chimeric poxvirus is administered at a cell number of 14×10⁶. In embodiments, the neural stem cell including the chimeric poxvirus is administered at a cell number of 15×10⁶. In embodiments, the neural stem cell including the chimeric poxvirus is administered at a cell number of 16×10⁶. In embodiments, the neural stem cell including the chimeric poxvirus is administered at a cell number of 17×10⁶. In embodiments, the neural stem cell including the chimeric poxvirus is administered at a cell number of 18×10⁶. In embodiments, the neural stem cell including the chimeric poxvirus is administered at a cell number of 19×10⁶. In embodiments, the neural stem cell including the chimeric poxvirus is administered at a cell number of 20×10⁶.

According to the methods provided herein, the subject is administered an effective amount of one or more of the agents provided herein. The terms effective amount and effective dosage are used interchangeably. The term effective amount is defined as any amount necessary to produce a desired physiologic response (e.g., treat cancer). Effective amounts and schedules for administering the agent may be determined empirically by one skilled in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary and can be administered in one or more dose administrations daily, for one or several days. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)).

For prophylactic use, a therapeutically effective amount of the neural stem cell including the chimeric poxvirus described herein is administered to a subject prior to or during early onset (e.g., upon initial signs and symptoms of cancer). Therapeutic treatment involves administering to a subject a therapeutically effective amount of the agents described herein after diagnosis or development of disease. Thus, in another aspect, a method of treating a disease (e.g., cancer) in a subject in need thereof is provided.

The terms “subject,” “patient,” “individual,” etc. are not intended to be limiting and can be generally interchanged. That is, an individual described as a “patient” does not necessarily have a given disease, but may be merely seeking medical advice.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition (e.g., cancer). For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

Regardless of how the chimeric poxvirus compositions are formulated, the dosage required will depend on the route of administration, the nature of the formulation, the nature of the subject's condition, e.g., immaturity of the immune system or a gastrointestinal disorder, the subject's size, weight, surface area, age, and sex, other drugs being administered, and the judgment of the attending clinicians. Alternatively or in addition, the dosage can be expressed as Pfu/kg of dry weight.

Administrations can be single or multiple (e.g., 2- or 3-, 4-, 6-, 8-, 10-, 20-, 50-, 100-, 150-, or more fold). The duration of treatment with any composition provided herein can be any length of time from as short as one day to as long as the life span of the host (e.g., many years). For example, a composition can be administered once a week (for, for example, 4 weeks to many months or years); once a month (for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

The compositions (e.g., a neural stem cell including a chimeric poxvirus) may also be administered in conjunction with other therapeutic agents. In embodiments, the compositions may also be administered in conjunction with anti-cancer agents. Other therapeutic agents will vary according to the particular disorder, but can include, for example, dietary modification, hemodialysis, therapeutic agents such as sodium benzoate, phenylacetate, arginine, or surgical remedies. Concurrent administration of two or more therapeutic agents does not require that the agents be administered at the same time or by the same route, as long as there is an overlap in the time period during which the agents are exerting their therapeutic effect. Simultaneous or sequential administration is contemplated, as is administration on different days or weeks.

The compounds described herein (e.g., a neural stem cell including a chimeric poxvirus) can be used in combination with one another, with other active agents (e.g. anti-cancer agents) known to be useful in treating a disease described herein (e.g. breast cancer, colon cancer, kidney cancer, leukemia, lung cancer, melanoma, ovarian cancer, prostate cancer, pancreatic cancer, brain cancer, liver cancer, gastric cancer or a sarcoma), or with adjunctive agents that may not be effective alone, but may contribute to the efficacy of the active agent.

In embodiments, the method includes administering a therapeutic agent. In embodiments, the therapeutic agent is a checkpoint inhibitor protein. In embodiments, the checkpoint inhibitor protein is B7-1, B7-2, PD-1, PD-L1, PD-L2, CTLA-4, CD40, CD40L, CD47, CD48, CD244, CD80, CD86, CD155, IDO, CDK-12, Galectin-9, LAG-3, TIM-3, VISTA, TIGIT, or SIRPα. In embodiments, the therapeutic agent is a small molecule, shRNA, siRNA, amtibody, minbody, diabody, triabody, nanobody or single domain antibody. In embodiments, the therapeutic agent is ipilimumab. In embodiments, the therapeutic agent is pembrolizumab. In embodiments, the therapeutic agent is nivolumab. In embodiments, the therapeutic agent is talimogene laherparepvec. In embodiments, the therapeutic agent is durvalumab. In embodiments, the therapeutic agent is daclizumab. In embodiments, the therapeutic agent is avelumab. In embodiments, the therapeutic agent is a chimeric poxvirus. In embodiments, the therapeutic agent is atezolizumab. In embodiments, the neural stem cell including the chimeric poxvirus and the therapeutic agent are administered at a combined synergistic amount. In embodiments, the neural stem cell including the chimeric poxvirus and the therapeutic agent are administered simultaneously. In embodiments, the neural stem cell including the chimeric poxvirus and the therapeutic agent are administered sequentially.

In some embodiments, co-administration includes administering one active agent within 0.5, 1, 2, 4, 6, 8, 10, 12, 16, 20, or 24 hours of a second active agent (e.g. anti-cancer agent). Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

In an aspect is provided a method of treating cancer, the method including administering to a subject in need thereof an effective amount of a chimeric poxvirus and a neural stem cell (NSC). For the methods and compositions provided herein the chimeric poxvirus provided herein forms part of a neural stem cell when administered. In embodiments, where a further therapeutic agent is administered, the therapeutic agent may be a chimeric poxvirus. Thus, in an aspect is provided a method of treating cancer, the method including administering to a subject in need thereof an effective amount of (i) a neural stem cell (NSC) including a first chimeric poxvirus, wherein the first chimeric poxvirus forms part of the neural stem cell, and (ii) a second chimeric poxvirus. The second chimeric poxvirus may be any one of the chimeric poxviruses described herein including embodiments, thereof.

In embodiments, the chimeric poxvirus forms part of the NSC. In embodiments, the poxvirus is encoded by a nucleic acid sequence having a sequence identity of at least 70% to SEQ ID NO:1. In embodiments, the nucleic acid sequence including nucleic acid fragments from at least two poxvirus strains selected from the group consisting of cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, vaccinia virus strain AS, orf virus strain NZ2 and pseudocowpox virus strain TJS. In embodiments, the nucleic acid sequence including nucleic acid fragments from cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic and vaccinia virus strain AS. In embodiments, the nucleic acid sequence further includes one or more anti-cancer nucleic acid sequences or a detectable moiety-encoding nucleic acid sequence. In embodiments, the chimeric poxvirus is a replication-competent chimeric poxvirus.

In embodiments, the NSC is a Human Leukocyte Antigen (HLA) II-negative NSC. In embodiments, the NSC is an HB1.F3.CD21 cell.

In embodiments, the method includes further administering an effective amount of a therapeutic agent. In embodiments, the therapeutic agent is an anti-cancer agent. In embodiments, the therapeutic agent is selected from the group consisting of a small molecule, a nucleic acid, a polypeptide and an antibody. In embodiments, the therapeutic agent is a PD-L1 inhibitor, a CTLA-4 inhibitor or an OX40 inhibitor. In embodiments, the therapeutic agent is a PD-L1 inhibitor. In embodiments, the therapeutic agent is a CTLA-4 inhibitor. In embodiments, the therapeutic agent is an OX40 inhibitor. In embodiments, the therapeutic agent is an anti-PD-L1 antibody, an anti-CTLA-4 antibody or an anti-OX40 antibody. In embodiments, the therapeutic agent is an anti-PD-L1 antibody. In embodiments, the therapeutic agent is an anti-CTLA-4 antibody. In embodiments, the therapeutic agent is an anti-OX40 antibody.

In embodiments, the composition is effective to treat cancer. In embodiments, the cancer is breast cancer, colon cancer, kidney cancer, leukemia, lung cancer, melanoma, ovarian cancer, prostate cancer, pancreatic cancer, brain cancer, liver cancer, gastric cancer or a sarcoma. In embodiments, the cancer is ovarian cancer. In embodiments, the cancer is metastatic cancer. In embodiments, the ovarian cancer is an ovarian metastasis.

In another aspect is provided a method of treating cancer, the method including administering to a subject in need thereof an effective amount of a neural stem cell as described herein, including embodiments. In embodiments, the cancer is breast cancer, colon cancer, kidney cancer, leukemia, lung cancer, melanoma, ovarian cancer, prostate cancer, pancreatic cancer, brain cancer, liver cancer, gastric cancer or a sarcoma. In embodiments, the ovarian cancer is an ovarian metastasis

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Neural Stem Cell-Delivered Chimeric Pox Virotherapy for Ovarian Cancer

Ovarian cancer is the most lethal gynecologic malignancy, afflicting approximately 22,000 women per year in the U.S with 14,200 deaths per year in the U.S. Once ovarian cancer has metastasized to the abdominal cavity (stage III), patients have only a 34% 5-year survival rate following standard treatment with surgical debulking and combination chemotherapy. Use of intraperitoneally (IP) delivered combination chemotherapy regimens has improved outcomes; however, these regimens frequently have complications and serious toxic side effects that prevent most patients from completing full treatment cycles. Additionally, most ovarian cancer patients eventually develop chemoresistance, leading to cancer progression and death. Replication-competent oncolytic virotherapy offers a new, highly promising approach for treating ovarian cancer. Once seeded into the tumor, the oncolytic virus can selectively replicate in tumor cells (but not in normal tissue) to destroy tumor cells in situ via direct lysis. Importantly, oncolytic viruses can induce cancer cell death irrespective of radio- or chemoresistance and can also stimulate immune system recognition of cancer cells by exposing tumor antigens upon lysis. Clinical efficacy of this approach has been limited by rapid viral inactivation by the immune system, poor viral penetration of tumors, and an inability of the virus to effectively reach invasive metastatic foci separated by normal tissue.

We now present a novel conditionally replication-competent chimeric orthopoxvirus (CF33) selected specifically for ovarian cancer cell infection, following screening of over 100 chimeric poxviruses. The CF33 virus was engineered to be tumor specific by deleting the virus's thymidine kinase (TK) gene. Tumor tissues have abnormally high TK expression that complements this gene deletion, permitting viral replication, whereas normal tissue does not. We have modified a tumor-tropic, HLA II-negative neural stem cell line (HB1.F3.CD21 NSCs; demonstrating clinical safety in high-grade glioma patients) that efficiently delivers CF33 to ovarian cancer foci in preclinical models^{[96]}. We demonstrate that NSCs provide protection from immune-mediated clearance and neutralization achieve effective viral distribution to peritoneal ovarian tumors. Co-culture in vitro experiments of NSC-delivered CF33 in human OVCAR8 and murine ID8 ovarian cancer cells showed robust infection of >95% of tumor cells in 6 days, even at a low ratio of 1 NSC:1000 tumor cells. In vivo data show selective distribution of NSC-delivered CF33 to human OVCAR8 and ID8 peritoneal metastases in immunodeficient and immunocompetent mice, respectively. Furthermore, IP-delivered CF33-NSCs showed increased viral distribution to tumor sites (assessed by BLI) compared to a matched viral load of free CF33 when multiple treatment rounds were given in the immunocompetent model. Long-term efficacy studies are in progress to establish the clearance kinetics, immune response, viral distribution, and efficacy of NSC-delivered vs. free CF33. Our long-term goal is to demonstrate efficacy and safety of CF33-NSCs for selective tumor killing in patients suffering from stage III ovarian cancer.

This cancer has an exceptionally high mortality rate, largely because the majority of patients present at an advanced stage. Use of intraperitoneal (IP) delivered combination chemotherapy regimens (relative to intravenous delivery) has improved outcomes, however, increased complications and toxic side effects are often brutal, with most patients unable to complete the treatment cycles due to severe abdominal pain, nausea, and vomiting. Therefore, new, more targeted and effective therapeutic approaches for treating recurrent and/or drug-resistant ovarian cancer are urgently needed. These findings motivate our need to establish the best schedule to deliver CF33 NSCs. Together, these preliminary data motivate further research because they suggest that CF33 NSCs can selectively penetrate peritoneal ovarian metastases, produce additional CF33 following IP administration and slow tumor progression.

Key results: 1. NSCs exhibit remarkable tropism to ovarian metastases; 2. CF33 NSCs cells are able to migrate to and kill ovarian cancer cells in vitro; 3. NSCs are able to deliver the CF33 virus to disseminated ovarian metastases in vivo and also slow its progression.

### Example 2

Conditionally replication-competent oncolytic virotherapy offers a new, highly promising approach for treating ovarian cancer. Once seeded into the tumor, the oncolytic virus selectively replicates only in tumor cells (but not in normal tissue) to destroy the cancer in situ via direct lysis.^{[2]} The lysed cells free additional viral particles that continue to infect neighboring tumor cells, amplifying their anti-tumor effect until normal tissue is reached and viral replication ceases.^{[4]} Oncolytic viruses induce cancer cell death regardless of whether the cancer is radio- or chemoresistant^{[5]} and can stimulate immune recognition of cancer cells^{[6]} by exposing tumor antigens upon cell lysis. Although clinical trials to date have demonstrated the safety of oncolytic viruses,^{[7]} their efficacy has been limited by delivery hurdles, such as rapid immune inactivation, poor penetration of tumors, and inability to reach invasive tumor foci that are separated from the main tumor mass by normal tissue.^{[8]}

We propose to overcome these barriers by using a clinically-relevant, safe, tumor-tropic neural stem cell (NSC) line (HB1.F3.CD), which can shield viruses from immune recognition en route to metastatic tumor foci. The NSCs will afford an unprecedented ability to amplify viral payloads in situ selectively in ovarian tumors.^{[4, 6, 9]} This approach will be tested using a novel oncolytic chimeric poxvirus, CF33, which replicates specifically in tumors.^{[2, 8, 9]} This specificity arises because CF33 includes a deletion of the gene encoding thymidine kinase, an enzyme needed for nucleic acid metabolism. Therefore, CF33 replication is dependent on cellular thymidine kinase expression, which is high in proliferating cancer cells but not in normal cells. Thus, this approach provides a safe, tumor-selective, immune-protective delivery method to improve oncolytic virotherapy.

Determine the optimal IP CF33-NSC dose for delivery of maximal viral load. We will determine the CF33-NSC dose that results in maximal delivery of CF33 chimeric orthopoxvirus to peritoneal ovarian metastases in an immunocompetent mouse model. Treatment will be completed with a range of CF33-NSC doses and tumors evaluated using qPCR and IHC to determine the dose that yields the most infectious virus particles in tumors.

Determine the clearance kinetics of CF33 in vivo. Clearance kinetics will be determined after IP injections of both free virus and CF33-NSCs using the experimentally determined dosage provided herein in an immunocompetent mouse model of peritoneal ovarian metastases. Tumors will be excised after treatment and analyzed by qPCR to quantify infectious virus particle clearance. Ninety percent reduction in the BLI signal compared to BLI signal 1 day after treatment will be considered the optimum time interval between repeat treatment rounds to maximize therapeutic effect.

Determine the therapeutic efficacy and anti-cancer immune response of CF33-NSCs versus free CF33 in vivo (+/− checkpoint inhibitors). We will conduct repeat IP administrations of CF33-NSCs or free CF33 in an immunocompetent syngeneic mouse model of peritoneal ovarian metastases, monitor tumor progression with bioluminescence imaging, and measure long-term survival. Here, PD-L1 blockade and CF33-NSCs will be combined as a potential treatment strategy for ovarian cancer. Because of its tumor-selectivity, we expect this therapeutic approach will increase long-term survival and reduce toxicities associated with current chemotherapies. These data will provide the basis for a pre-IND submission and IND-enabling studies. This approach may also be applicable to other high thymidine kinase-expressing cancers, such as breast and lung cancer.^{[10,11]}

Research Strategy

A. Significance

Clinical need. Each day, 60 American women are diagnosed with ovarian cancer, equating to nearly 22,000 women afflicted per year. Despite decades of research efforts, ovarian cancer continues to be the most lethal of gynecologic malignancies,^{[1, 2]} largely because the majority (75%) of patients present at an advanced stage (stage III), with widespread metastatic disease within the peritoneal cavity.^{[12]} The median overall survival for these patients is less than 3 years after standard treatment of intravenous or intraperitoneal (IP) administered combination chemotherapy (e.g., cisplatin and paclitaxel)[3]+/−surgical debulking. Quality of life for patients undergoing chemotherapy is poor and associated with such severe side effects, including abdominal pain, nausea, and vomiting, that most patients are unable to complete treatment.^{[13, 14]} Additionally, most ovarian cancer patients eventually develop chemoresistance and succumb to their disease within a few years. This sobering clinical scenario underscores an urgent critical need for new, more effective therapies that can improve both quality of life and treatment outcomes for patients with drug refractory ovarian cancer.

Oncolytic virotherapy for ovarian cancer. Conditionally replication-competent oncolytic virotherapy is a highly promising approach for treating ovarian cancer. In this novel approach, viruses are modified to preferentially replicate in tumor cells and selectively destroy them in situ via direct lysis.^{[2]} The viral particles freed from lysed tumor cells continue to infect neighboring tumor cells, amplifying their anti-neoplastic effect until normal tissue is reached, causing viral replication to cease^{[4]}. Important for treating cancer, oncolytic viruses have the ability to induce cancer cell death irrespective of radio- or chemoresistance^{[7]} and can stimulate immune recognition of cancer cells^{[6]}. More than 20 oncolytic viruses have been developed so far, and 11 have been tested in pre-clinical human ovarian cancer models^{[2]}. To date, four of these 11 viruses have been tested in 9 phase I/II clinical trials^{[2]}. Most generations of oncolytic viruses currently in human testing were engineered to have highly attenuated activity. Thus, although these clinical trials are still in early stages, they have all established the safety/non-toxicity of this approach^{[7]}, and because toxicities are so low, not a single oncolytic virus trial has established a maximum tolerated dose^{[2]}. However, for the same reason, few trials have produced major responses to therapy. Therefore, the approach taken by most groups engineering new immuno-oncolytic viruses is to make viruses that are much more potent at specifically killing cancer cells. Designing delivery vehicles for these novel viruses could further increase the amount of virus delivered to sites of cancer while minimizing side-effects.

Oncoviral therapies could theoretically offer a “one-shot” cure, with the viral particles amplifying their antineoplastic effect until normal tissue is reached.^{[4]} Unfortunately, the viral particles are susceptible to rapid inactivation and clearance before they are able to infect tumor cells. To realize the potential of oncolytic therapies for treatment of advanced cancer, dramatically improved delivery approaches are needed so that the viruses can persist without depletion or degradation. It is also clear that clinical application of immuno-oncolytic viruses will require repeated administration of the viruses, which results in greater host immune system inactivation. The main strategies to overcome the antiviral immune response have been to co-deliver the virus with immune-suppressive drugs (e.g., cyclophosphamide or anti-CD20) or to physically shield the virus from immune recognition (e.g., with PEGylated liposomes or cell carriers).^{[15-17]}

Tumor-tropic stem cells for oncolytic virus delivery. Drawbacks to current methods include recognition by the immune system, poor penetration and distribution through metastatic tumor foci, and limited amplification of viral payloads in situ.^{[4,6,8]} Combining tumor-specific oncolytic viruses with tumor-tropic cells offers potential advantages over free virus or liposome-coated viral delivery (FIG. 21). The first experiments combining viral particles with cell delivery vehicles attempted to passively bind viruses to the surfaces of tumor-tropic T-cells.^{[18]} However, most virus was neutralized before it reached the tumor, suggesting that carrier cells must be transduced with the virus. Because the parameters that control viral uptake efficiency in various cell types are still being discovered, only certain combinations of cells and viral payloads are currently feasible^{[19]}. The ideal cell carrier for oncolytic virus delivery should be chromosomally stable, support viral infection and amplification, shield the virus from the host immune system while en route to the target, and have an intrinsic tumor-homing ability to deliver and seed virus directly to sites of metastases.

This NSC delivery strategy offers the following advantages over free virus and immune-based cellular delivery vehicles: 1) NSCs express minimal human leukocyte antigen (HLA) class I and no class II molecules, and can shield the virus from the immune recognition en route to tumor sites; 2) NSCs enable improved penetration and distribution of the virus within tumor sites and to invasive metastases; and 3) NSCs afford an unprecedented ability to amplify viral payloads in situ^{[4]}. Thus, NSCs provide a safe, tumor-selective, immune-protective delivery vehicle to improve the efficacy of oncolytic virotherapy.

Therapeutic candidate: Chimeric orthopoxvirus-producing NSCs. We focus on a novel chimeric orthopoxvirus (CF33) engineered for high potency against ovarian cancer. The CF33 virus is designed to have a favorable safety profile by being conditionally replicative in cancer cells, but not in normal somatic cells. This selectivity has been engineered by deleting the virus's thymidine kinase (TK) gene. Tumor tissues express abnormally high levels of TK, which will permit viral replication. In contrast, normal tissue does not express sufficient levels of TK to allow viral replication.^{[12]} We have engineered our HB1.F3.CD21 NSCs to produce CF33^{[21]} (CF33-NSCs). In studies, the efficacy and safety of HB1.F3.CD21 NSCs engineered to express an oncolytic adenovirus (CRAd-Survivin-pk7) in preclinical brain tumor models have been demonstrated.^{[8,22,23]} These studies support initiation of a phase I clinical trial of the CRAd-S-pk7-NSC approach for newly diagnosed glioma patients (NCT03072134). CF33-NSCs described herein therapeutically surpass previous CRAD-S-pk7 NSCs, because compared to adenoviruses, oncolytic poxviruses have greater viral spread, tumor infection, and cytoplasmic productive replication in tumor cells with no host genome integration and recombination.^{[24,25]}

CF33 stimulates the expression of PD-L1, an immune checkpoint protein that blocks recognition by immune effector cells, in both tumor and tumor-associated cells. Therefore, we plan to combine CF33-NSCs with administration of a PD-L1 inhibitor.^{[26]}

In these studies, NSCs are transduced with CF33 virus and frozen into aliquots that are thawed and rinsed just prior to administration per clinical SOPs. CF33-NSCs are then injected IP into mice with peritoneal ovarian metastases. NSCs will selectively target and penetrate tumor metastases, effectively delivering and distributing the CF33 virus. The virus will replicate selectively within tumor cells until they lyse and release virus to surrounding tumor, amplifying its therapeutic effect until normal tissue is reached. IP-administered CF33-NSCs will selectively penetrate IP ovarian metastases and significantly improve clinical outcomes (i.e., decrease tumor burden and/or increase long-term survival) as compared to the free CF33 virus. The methods and compostions provided herein will significantly impact ovarian cancer; it has the potential to both increase long-term survival for stage III ovarian cancer patients and reduce toxicities associated with current therapies. In addition, the proposed CF33-NSCs will serve as an effective stand-alone and/or adjuvant treatment that increases the therapeutic index of current chemotherapeutic regimens. Furthermore, although the proposed study focuses on ovarian metastases, this CF33-NSC platform will be easily translated to other types of peritoneal carcinomas, regardless of their anatomical origin. Upon further development, this platform could also improve outcomes for other peripheral metastatic tumors that overexpress TK (e.g., breast and lung cancers). NSCs may also be capable of improving peritoneal delivery of other therapeutic viruses (e.g., those expressing anti-inflammatory genes to avoid dialysis-induced peritonitis).^{[27, 28]}

B. Innovation

Mesenchymal stem cells (MSCs) have been widely tested in preclinical studies as delivery vehicles for oncolytic viruses and are being tested as protectors of viral cargo in an ongoing clinical trial.^{[29]} However, there are major drawbacks to the use of MSCs, including that they are composed of very heterogeneous cell populations, have poor ex vivo loading capacities, and lose their tumor-homing properties after 5-6 passages.^{[30]} Moreover, the amount and quality of autologous MSCs that can be isolated depends on patient age and current health status, and it can take several weeks to generate enough cells for treatment.^{[30]} For example, Mader et al. recently demonstrated that it took 2 weeks after isolation to generate enough MSCs needed for treatment,^{[6]} 20% of which showed abnormal karyotypes.^{[30]} This is concerning because confirmed non-tumorigenicity of any stem cell therapy is of paramount importance. Although MSCs may represent a feasible cell-delivery vehicle for smaller phase I trials, overall they represent an inefficient and poorly reproducible strategy that likely will not pass the regulatory hurdles and scale-up required for phase II or III trials.

Here, in a departure from work using patient-derived MSCs, we instead propose to use allogeneic, immortalized HB1.F3.CD21 NSC line^{[31]} to deliver oncolytic orthopoxviruses. This line is chromosomally and functionally stable, and has demonstrated clinical safety, non-immunogenicity, tumortropism, and proof-of-concept for tumor-localized chemotherapy production.^{[32]} Demonstrated safety and stability, as well as the practicality and cost-effectiveness of an ‘off the shelf’ product are major advantages of using an established allogeneic cell line for delivery, in contrast to the difficulties and uncertainties of using isolated primary autologous cells. Specifically, HB1.F3.CD21 NSCs will be used to produce CF33, a novel chimeric orthopoxvirus that selectively replicates in tumor cells that overexpress TK. By using cells as tumor-selective, immune-protective delivery vehicles, target viral payloads will be delivered to invasive micrometastatic tumors^{[4]} that are separated from the main tumor mass by normal tissue.^{[33]} This has not yet been possible. Our combination therapy using NSCs to deliver a novel chimeric orthopoxvirus (CF33-NSCs) has never been applied to any metastatic cancer before. The proposed research is a new and substantive departure from the status quo of using patient-derived MSCs to deliver oncoviral therapies within the peritoneal space. In addition, the use of IP-administered CF33-NSCs is the first application of NSC-oncolytic virotherapy for ovarian cancer in patients. Applicants have been the first to use NSCs to deliver therapeutics within the peritoneal cavity, and Applicants' preliminary studies (see Approach) strongly suggest that this approach will be clinically effective. In addition, production of CF33-NSCs will be more efficient and economical than production of patient-derived MSCs because our NSC platform is established as a Master Cell Bank (MCB) at the City of Hope Biological and Cellular GMP Manufacturing Facility and can be expanded, modified, tested, and banked for an “off-the shelf” product readily available for trials at multiple sites. We have now established cell manufacturing and patient preparation standard operating procedures (SOPs) supporting the feasibility of rapid clinical translation of positive results.

C. Approach

Preliminary Studies

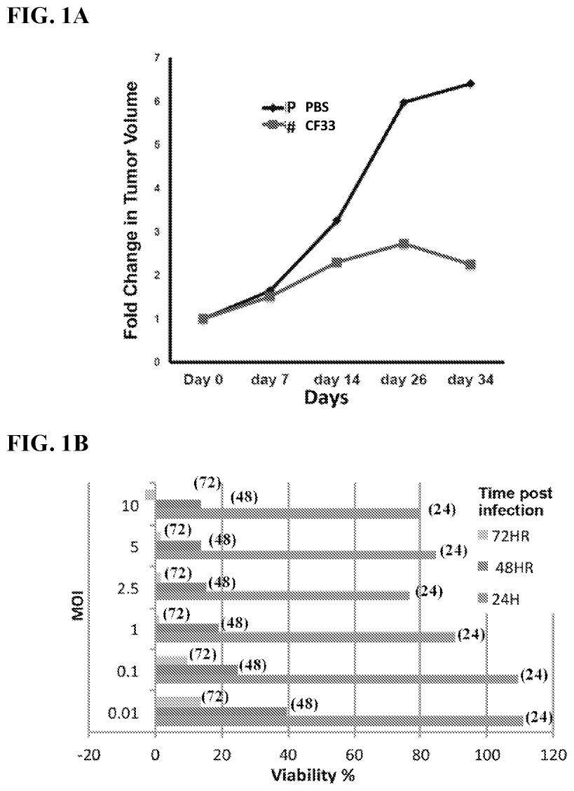

CF33 chimeric orthopoxvirus and CF-33 NSCs. Applicants generated, screened, and evaluated novel oncolytic chimeric poxviruses that were highly selective to ovarian cancer (FIG. 1A). Following screening of more than 100 chimeric poxviruses, several chimeras showed superior cancer cell killing in NCI-60 cell lines and were further evaluated. Human OVCAR8 ovarian cancer cells were infected with various multiplicities of infection (MOIs) and assessed for % survival. Based on results (not shown) chimera 33/TK- (CF33) was chosen as an effective therapeutic agent for further studies, including delivery by the HB1.F3.CD21 NSC line. An advantage of using an immortalized, clonal NSC line for oncoviral delivery is that viral transduction and burst kinetics are highly reproducible. We have determined the optimum viral loading into NSCs to be 3×10⁶ PFU per million NSCs, as this amount of viral loading confers approximately 1 MOI per cell, does not alter NSC differentiation status or tumor-tropism in vitro. Cell-associated viral counts reached a maximum at 24 h postinfection, at which point the NSCs lysed (FIG. 1B).

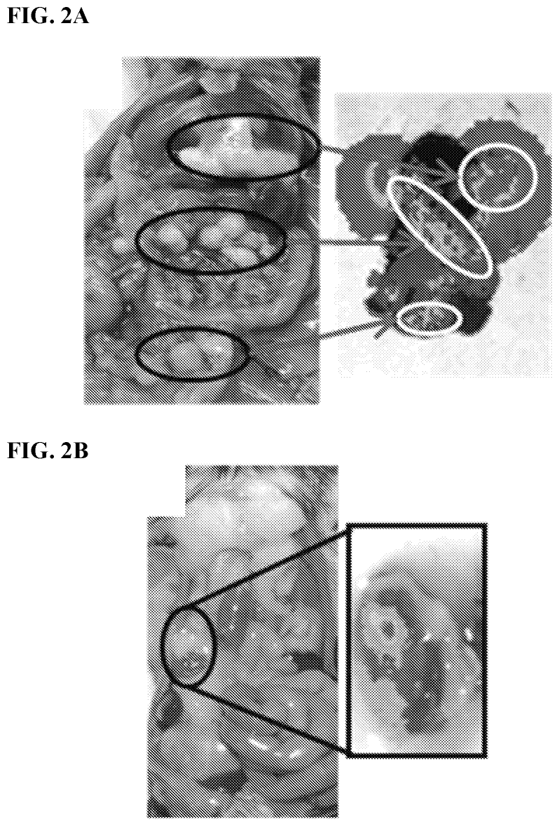

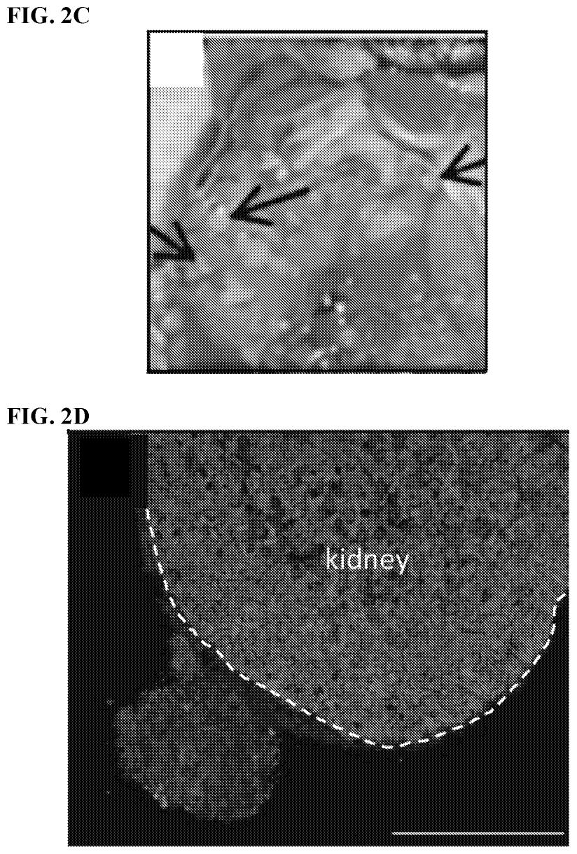

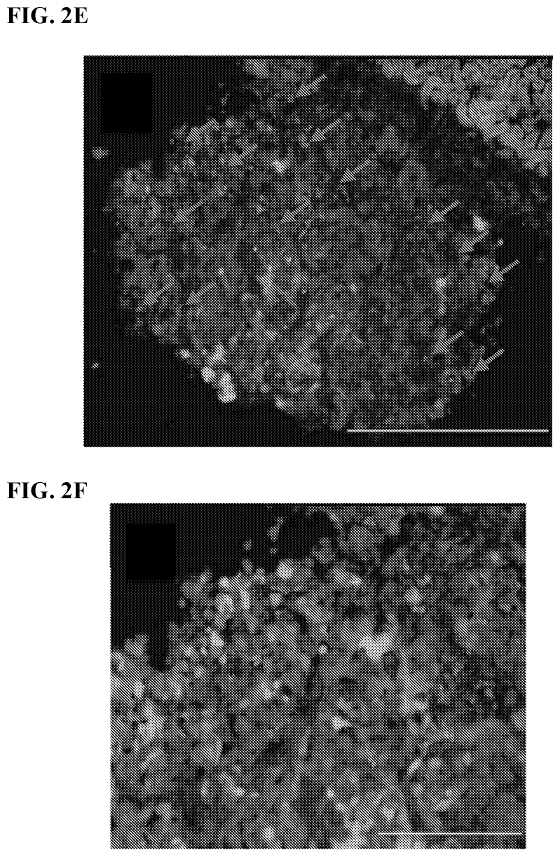

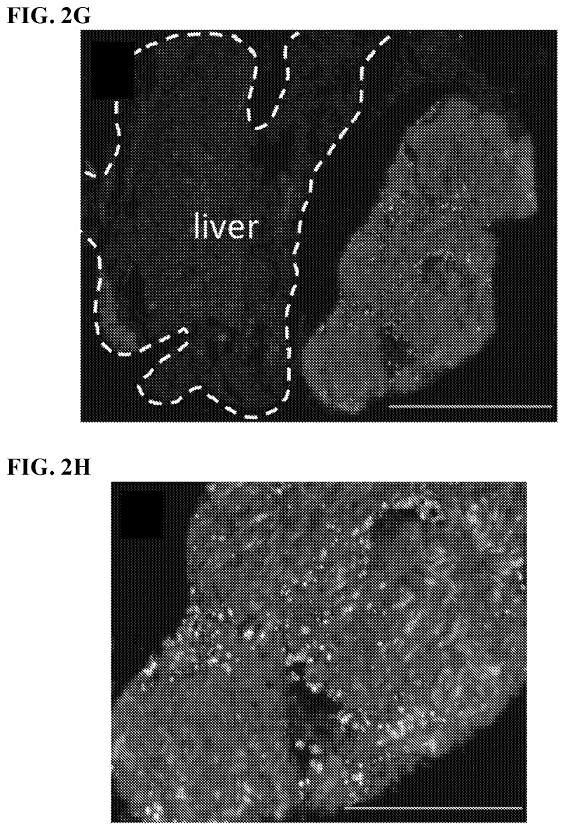

NSC tropism to peritoneal ovarian cancer metastases models. Ovarian cancer cells typically spread from the primary tumor site into the ascites fluid of the peritoneal space, followed by secondary tumor seeding onto the serosal surfaces of abdominal organs.^{[39]} We generated immunodeficient and syngeneic immunocompetent mouse models of ovarian cancer by inoculating ovarian cancer cells into the peritoneal cavity of nu/nu and C57Bl/6 mice, respectively. High-grade serous ovarian cancer lines were selected and fluorescently labeled (human OVCAR8.egfp/ffluc and murine ID8.dt.tomato). We chose serous ovarian cancer because it is the most frequent histotype of cancer in patients diagnosed with stage III peritoneal disease. Tumor cells seeded predominantly to the greater omentum and serosal surfaces of the liver, kidney, intestines, diaphragm, and pancreas. Tumor growth kinetics and distribution were determined post-IP injection of 10×10⁶ tumor cells (FIG. 2A-2H). We saw tropism of CD-NSCs to tumors in both models.^{[41]} It was also demonstrated that the CF33 orthopoxvirus effectively infects both human and mouse ovarian tumor cells in vitro and in vivo. We selected the immunocompetent model for our CF33-NSC dosing and efficacy studies, described herein, as it permits analysis of the contribution of the immune system on the tumor microenvironment in response to treatment.

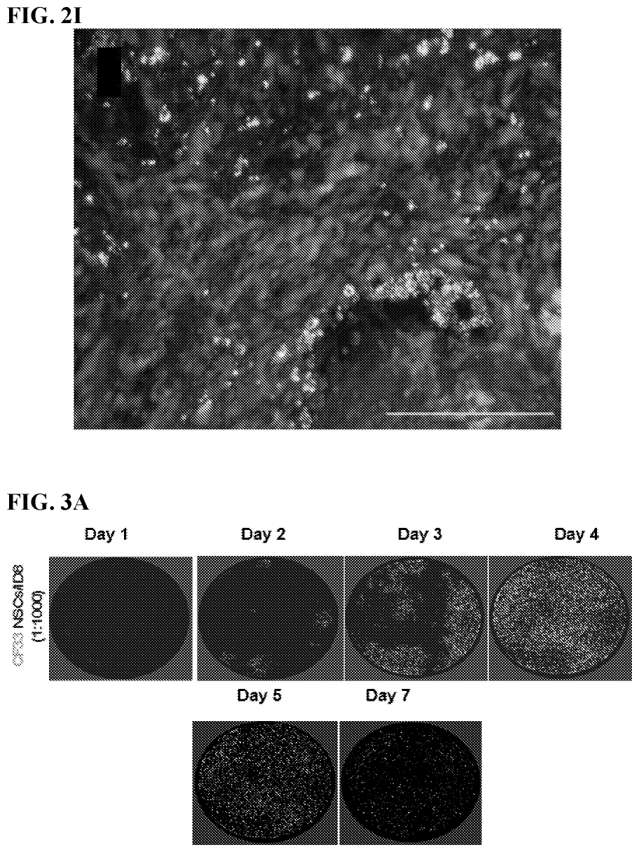

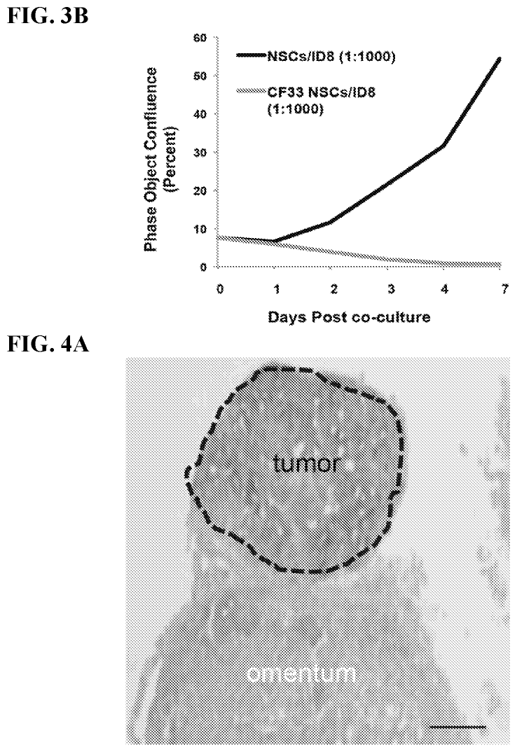

Rapid in vitro infection of ovarian cancer cells with CF33 virus delivered by NSCs. The poor delivery efficiency of standard viral vectors necessitates potent bystander killing effects capable of destroying tumor cells that the administered virus cannot reach. We found that CF33 progeny released from CF33-NSCs produced a considerable local bystander effect against the ID8 ovarian cell cancer line. In vitro co-culture of ID8 cancer cells with CF33-NSCs (tumor cell:NSC ratio of 1000:1) resulted in replication of CF33 in ovarian cancer cells, as shown by amplification of the eGFP signal that progressed until the cells died (FIG. 3A). This was confirmed by analysis of confluency mask of phase images (FIG. 3B) and this indicates the CF33 virus can infect and amplify in neighboring tumor cells.

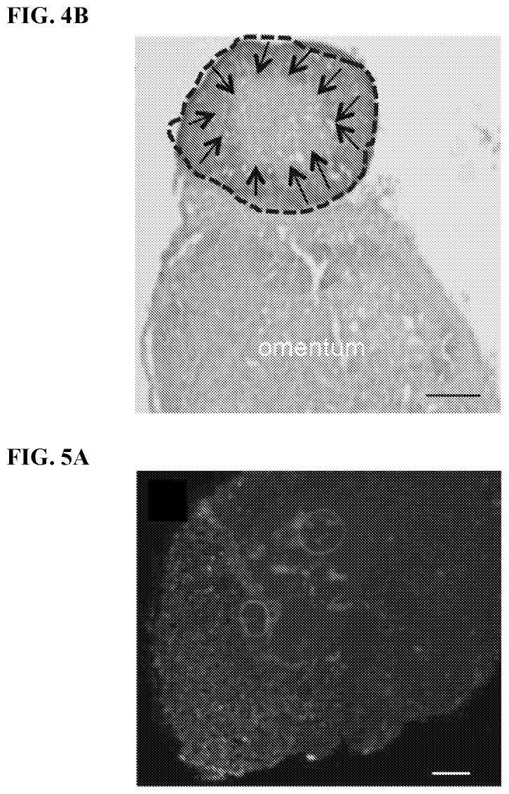

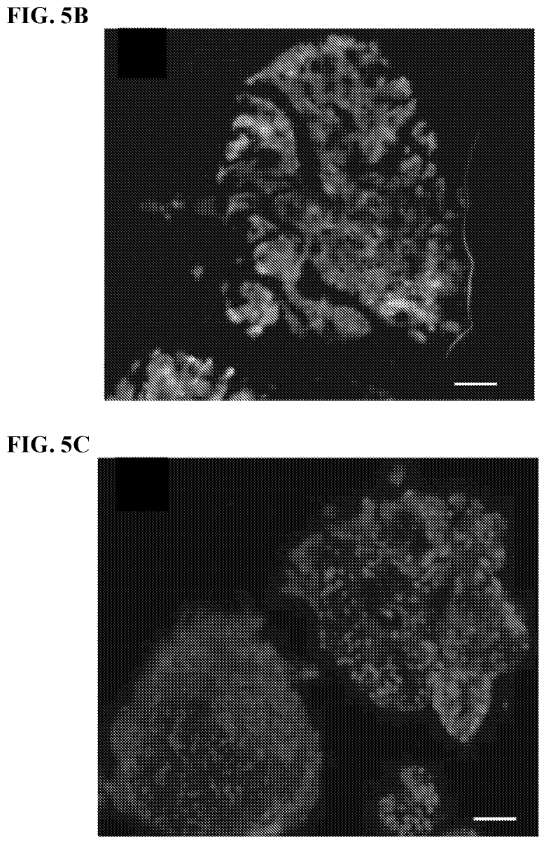

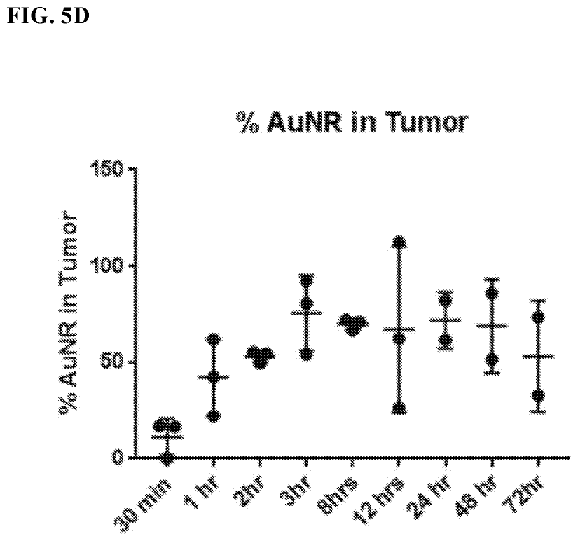

CF33-NSC delivery of virus selectively to ovarian metastases. We have demonstrated the tumor tropic properties of NSCs in preclinical models of invasive and metastatic tumors, including brain tumors,^{[37, 38]} breast carcinoma,^{[39]} metastatic neuroblastoma,^{[40]} and most recently peritoneal ovarian metastasis (NSCs delivered IP).^{[41]} We have also found that viral loading does not impair the tropism of NSCs to tumor metastases within the peritoneal cavity and that NSC-delivered virus spreads into tumors. The initial experiment was in the OVCAR8 nu/nu immunodeficient mouse and NSCs were not dectected in non-tumor tissue. (FIG. 4A, 4B). To confirm transfer of virus to tumor foci in an immunocompetent model, we treated C57Bl/6 mice with established IP ID8 tumors with CF33-NSCs, CF33 (matched viral load), or PBS control (FIG. 5A, 5B, 5C). Tumors were harvested 4 days after NSC administration and fluorescent immunocytochemistry using anti-vaccinia Ab performed on tumor cryosections.

NSCs localize to ovarian tumor foci within 1 hour post IP administration. In studies looking at NSC distribution in ovarian cancer models, we conjugated the NSCs to gold Nanorods (AuNR) for quantification of gold localized to tumor foci with ICP mass spectroscopy. No significant difference was observed in gold delivered by the NSCs at tumor foci between 1 hour and 72 hour post IP NSC administration (FIG. 5D).

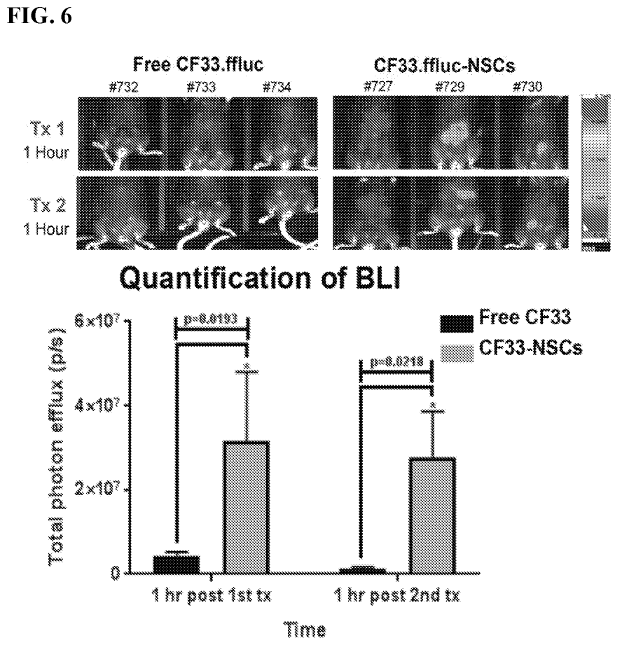

IP Administration of CF33-NSCs resulted in enhanced virus delivery to ovarian cancer metastases as compared with free CF33. To test if IP administered CF33-NSCs could deliver live CF33 to IP ovarian metastases, we conducted a pilot study (n=3 per group). C57Bl/6 with established ID8 IP metastases were treated with a matched viral load of either free CF33 or CF33-NSCs (CF33 labeled with ffluc; viral load 3×10⁶ CF33 PFU in 2×106 NSCs). In vivo bioluminescence imaging (BLI) for virus-specific gene expression (ffluc) was performed one hour after treatment (FIG. 6. Tx 1). Mice that received CF33-NSCs (right) showed significantly stronger signal than did those that received free CF33, after the first round of treatment (p=0193). This suggests improved delivery and distribution of virus to tumor foci. Mice then received a 2nd treatment one week later, and BLI was performed after 1 hour (FIG. 6, Tx 2), showing that the Signal remained significantly higher in mice receiving CF33-NSCs as compared to free CF33 (p=0.0218) which had no signal, likely due to immune inactivation of the free virus. These data support demonstrate that viral delivery by NSCs will provide an advantage over free virus by 1) improving penetration and distribution of virus through tumor foci, and 2) upon repeat treatment rounds, potentially shield virus from neutralization by the immune system en route to the tumor. To better investigate if clearance is mediated by immune clearance, the proposed studies will include weekly sampling of blood to assay immune response [e.g., antibody neutralization and cytokines] after each CF33-NSC or free CF33 administration.

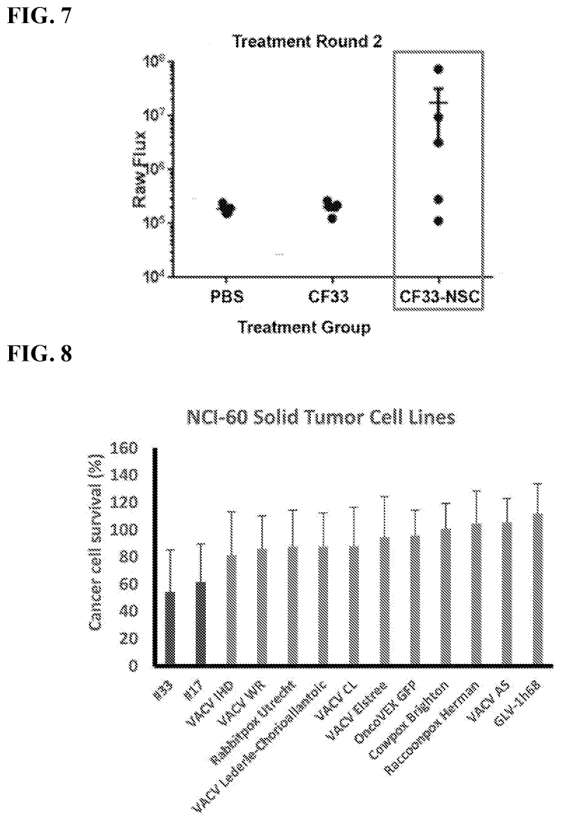

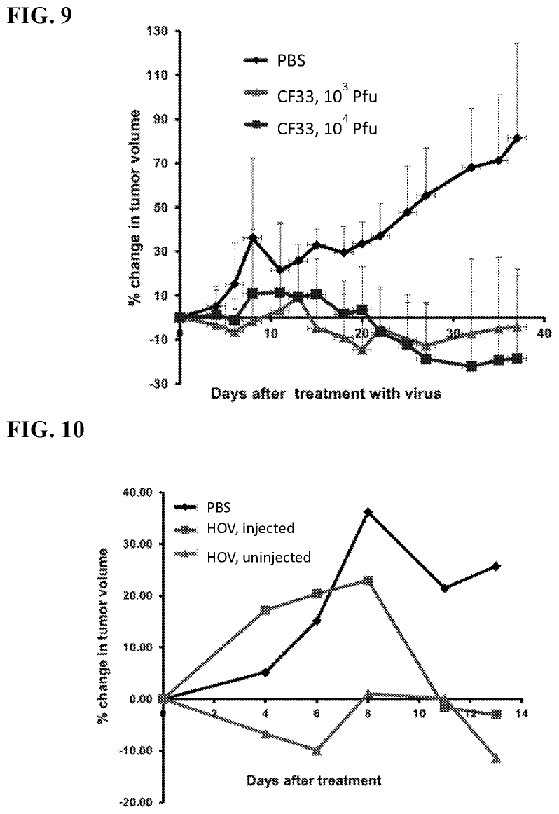

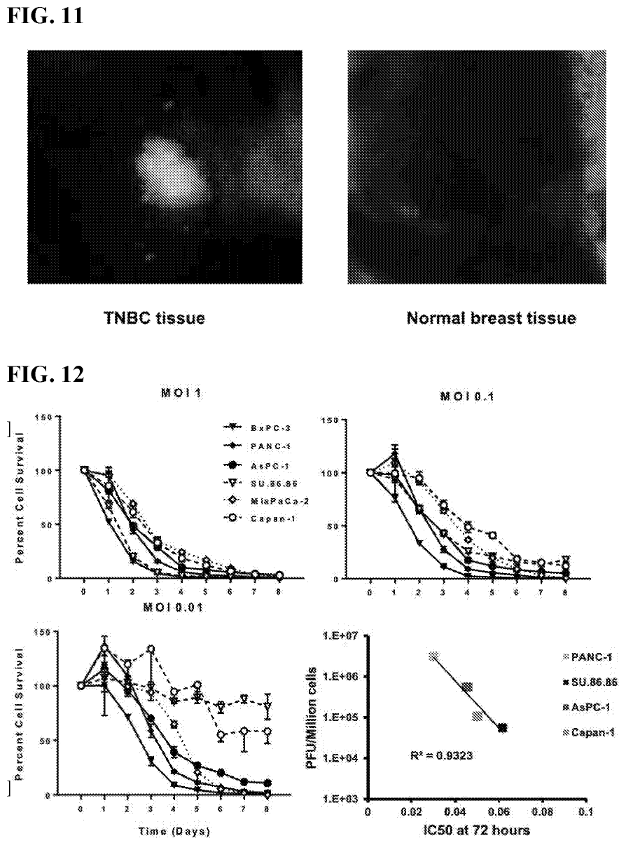

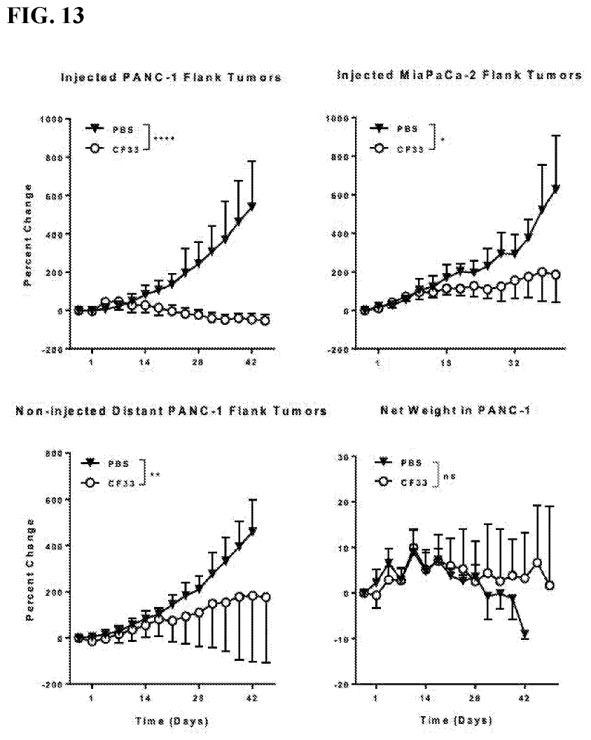

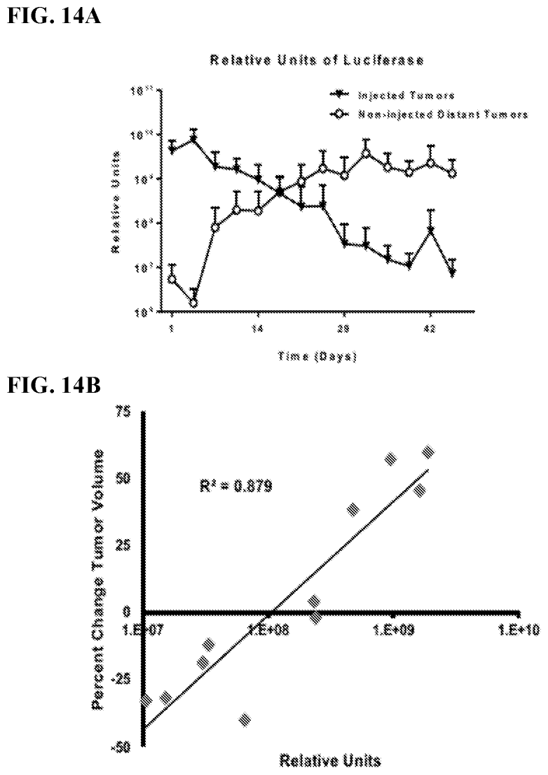

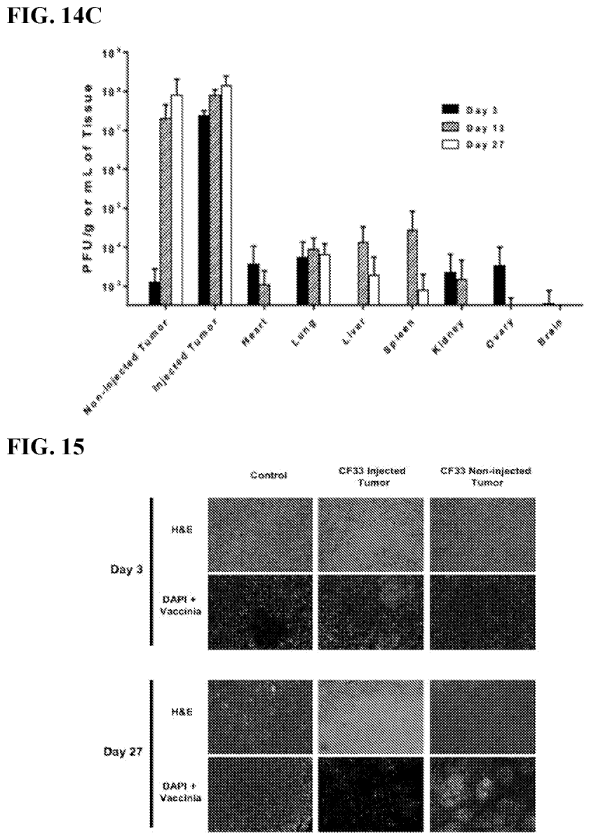

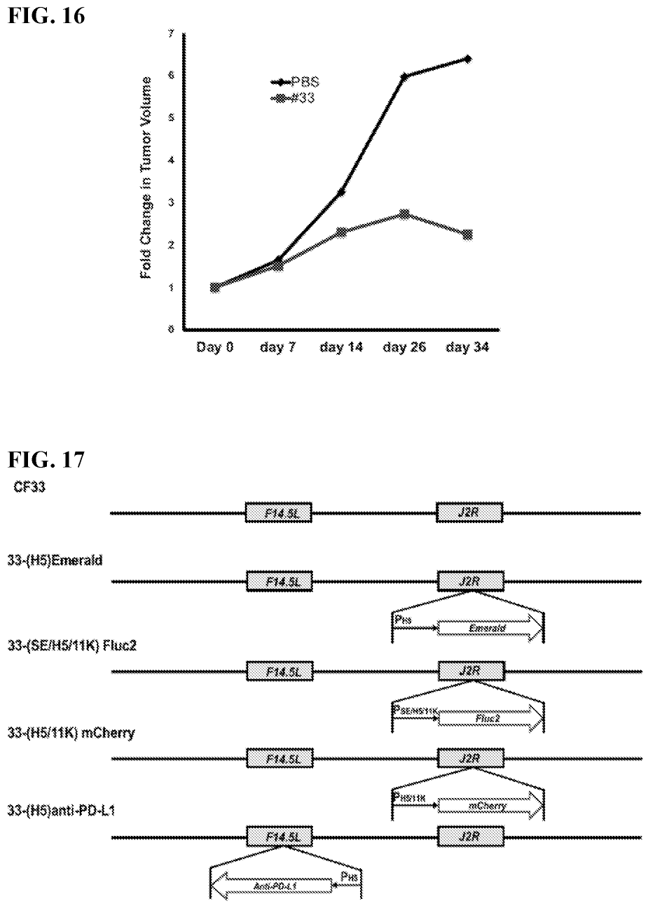

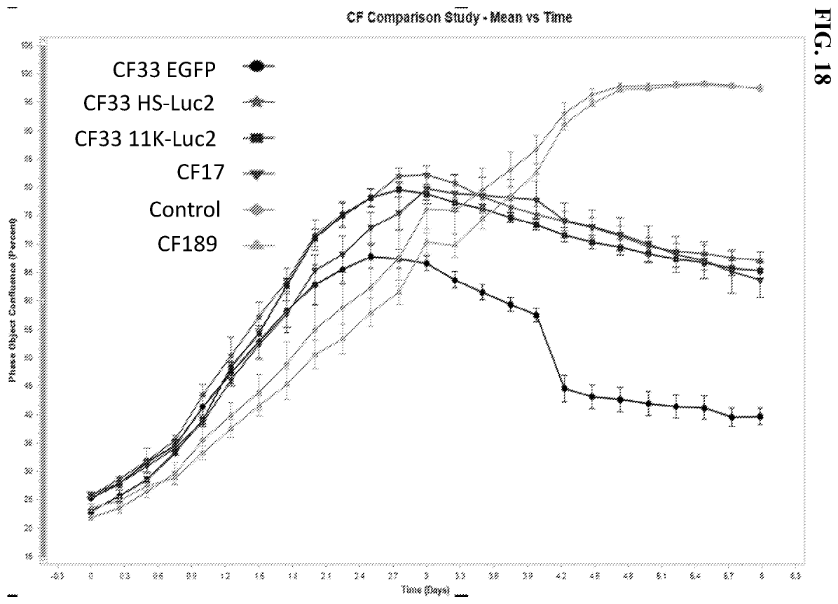

In a similar pilot study, C57Bl/6 immunocompetent mice (n=5 per group) with established ID8 ovarian metastases were treated with either free CF33 or CF33-NSCs (matched viral load, 3×10⁶ PFU/mouse). Mice treated with PBS served as a negative control. In this study, the 2^{nd} treatment was given 3 weeks after the initial treatment. Whole animal BLI was performed 1 day post-treatment for distribution of the virus within the peritoneum. Presence of virus in individual tumors isolated at harvest was confirmed using BLI (FIG. 7). Again, data suggest the immune system immediately inactivated and cleared the free virus, as no viral signal was detected in mice given free CF33 on day 1 post 2nd treatment. In contrast, 3 out of 5 mice that received CF33-NSCs had a detectable signal at day 1. We will more extensively investigate immune clearance of the virus over time, with daily in vivo whole body BLI and sampling of blood for analysis of immune and cytokine profile.

Collectively, these preliminary data support the feasibility of the proposed work, confirming the ability of NSCs to selectively target and deliver the CF33 oncolytic virus to peritoneal ovarian metastases in vivo and our ability to monitor its presence using established techniques.

Determine the Optimal IP NSC Dose for Delivery of Maximal CF33 Viral Load.

Introduction: Our preliminary studies demonstrate 1 hour after 2×10⁶ CF33-NSCs are injected IP, most macroscopic tumors contain at least some NSCs. It is not yet known if administering a greater number of CF33-NSCs, or perhaps administering fewer CF33-NSCs more frequently, would result in better tumor coverage. The dosing strategy that maximizes the initial localization of CF33 NSCs in peritoneal metastases is determined. We will test the working hypothesis that multiple, lower dose injections will be the most efficient strategy to achieve maximal CF33 delivery to IP metastases. In our studies of intravenous NSC administration in various tumor models, it was shown that dividing a single high dose into multiple lower dose administrations result in a greater number of NSCs localized to tumor sites. NSC-mediated delivery of CF33 to IP metastases will be assessed using standard biochemical and immunological assays. Establishing the proportional relationship between numbers of tumor-localized NSCs and the CF33 levels initially delivered to IP metastases is critical for fully designing the viral amplification and clearance studies planned. The dosing strategy determined herein will be used for determining clearance kinetics and therapeutic efficacy and anti-cancer immune response of CF33 vs CF33-NSCs in vivo.

Given these challenges, our approach to maximize viral load at disseminated metastases will be to maximize the number of CF33-NSCs present per tumor. One advantage of NSC-mediated viral delivery is that consistent NSC transduction efficiencies from batch to batch and consistent viral release times from transduced NSCs can be confirmed. Thus, we expect to observe a directly proportional relationship between the number of tumor-localized NSCs and CF33 viral particles. This controlled approach is not possible for other studies that deliver viral payloads using patient-derived MSCs, because transduction efficiencies and burst times vary significantly from patient to patient, isolation to isolation.

Research Design: All mouse studies will use female mice, as ovarian cancer is a female-specific disease Immunocompetent C57Bl/6 8-12 week old female mice will be inoculated IP with ID8 syngeneic ovarian tumor cells expressing Renilla luciferase (ID8RLuc). As per previous growth kinetics studies of this model, we expect that by 3 weeks after inoculation mice will have established a few macroscopic and numerous (>20) microscopic tumors, ensuring an adequate number of metastatic tumors available for analysis. BLI will be used to confirm tumor engraftment. Experiment 1.1: Mice bearing confirmed tumors will be IP injected with DiI-labeled CF33-NSCs (0.1×10⁶, 2×10⁶, or 4×10⁶ cells; 10 mice per dose). Twenty-four hours later, mice will be euthanized to determine NSC presence within metastatic tumors after a single administration. Experiment 1.2 Dil-labeled CF33-NSCs (2×10⁶ cells) will be given as either 1, 2, or 4 separate administrations within an 8-hour period (10 mice per administration group) and tumors will be collected 24 hours after the final administration to determine if a greater number of NSC administrations results in more efficient delivery of CF33. For both experiments 1.1 and 1.2, the location, size, and weight of macroscopic tumors will be recorded (Table 1). Ten tumors per mouse will be processed for qPCR quantification of the NSC-specific gene v-myc, of which there is 1 copy per NSC, to determine the number of NSCs present at each tumor. The number of NSCs at each tumor will be correlated with viral load, as assessed using qPCR for vaccinia gene expression and viral titration assays. The remaining tumors will be sectioned and stained with human specific nestin and antivaccinia antibodies to visualize NSC and viral co-localization.

Biostatistical analysis: The primary outcome of these experiments is the delivery of maximal virus load, which will refer to as virus retention hereafter. Virus retention will be compared among the various NSCs doses (Experiment 1.1) and numbers of injections (Experiment 1.2) (Table 1). Using a chi-square test with a 0.05 one-sided significance level, we will achieve statistical power of at least 85% to detect the difference between a dose with virus retention of 0.7 and a dose with retention of 0.15, when the sample size in each group is 10. This sample size justification also applies to Experiment 1.2 for one administration (virus retention of 0.70) vs. separate administrations (virus retention of 0.15). Multiple comparisons will not be corrected because of the relatively small sample size. Success Criteria: Identification of a CF33-NSC dose level (and number of injections) that increases virus retention at IP metastases by at least 50% compared with 0.1×10⁶ NSCs.

Expected Outcomes: Injecting 2×10⁶ CF33-NSCs in preliminary studies may be wasteful, as less than 10% of NSCs were detected within tumors 1 day after injection. Thus, we expect to find a lower dose, such as of 0.1×10⁶ CF33-NSCs could saturate the number of NSCs localized to IP metastases on day 1. Likewise, we expect that splitting this dose into multiple injections over the course of 8 hours will increase the number of NSCs at the tumor foci. This expectation is based on our data in which a given dose of NSCs administered intravenously was given as a single dose or divided into 2 or 3 doses given multiple times within 24 hours, resulting in an increase of NSCs at the tumor sites of 4-8-fold.

Potential Pitfalls and Alternatives: Although we expect to identify an NSC dose at which the number of tumor-localized NSCs saturates, none of the tested doses may change the number of tumor-localized NSCs. In this unlikely event, we would consider that the dose range used is too high, in which case, experiments will be repeated with lower doses. Although it is expected that 2 administrations will maximize the NSC distribution to IP metastases, 4 administrations may be even more effective. If this is the case, we will repeat the study with multiple administrations until a dosing strategy that results in maximal NSC coverage of IP metastases is identifed as determined by qPCR for v-myc (1 copy per NSC) and/or qPCR for CF33.

In an attempt to optimize the dosing strategy for human tumors, we could use mice reconstituted with human immune systems instead of immunocompetent mice. However, several mouse organs (e.g., bone marrow mesenchymal cells and endothelium) cannot interact effectively with grafted human leukocytes due to species specificity, and the interaction between mouse gut flora and the human immune system may be paradoxical. In addition, grafting is often performed utilizing an identical batch of stem cells in highly inbred animals, which fails to account for human heterogeneity. Limiting factors also include the substantial cost and restricting supply of animals.^{[44]} As an alternative to NSC-mediated CF33 delivery, we may test the efficacy of CF33 packaged in nanoparticles (NPs). However, only a few liposomal and protein-based NPs have been approved for the delivery of cancer drugs, including Doxil and Abraxane, and these NPs are passive targeting delivery agents that rely on the enhanced permeability and retention (EPR) effect for tumor localization.^{[45,46]} Liposomal NPs are limited by particle instability, rapid clearance, and spontaneous membrane fusion with off-target cells.^{[47,48]} Polymer-based NPs suffer from structural heterogeneity, particle instability, slow and non-uniform drug release, and potential immunogenicity.^{[49]} More stable metal-based NPs suffer from a lack of specificity and high toxicity.^{[50]} In addition, most of these NPs are limited by clearance mediated by phagocytes and dendritic cells, including Kupffer cells in the liver. Coating NPs with polyethylene glycol (PEG) can help avoid phagocytes and extend the blood circulation time by creating “stealth” brushes^{[51]}, 18; however, PEGylation can also reduce NP uptake by the targeted cells and is potentially immunogenic.^{[52,52]} Finally, surface functionalization of these NPs is difficult to control and non-uniform.^{[53]}

Determine the Clearance Kinetics of CF33 In Vivo.

Introduction: We expect that after IP injection, CF33-NSCs will home to tumor sites and begin amplifying viral progeny until the NSCs burst and transfer the viral payload to neighboring tumor cells. The virus should continue to amplify throughout the tumor, but many viral particles will also be cleared, eventually leaving minimal active virus within the abdomen. The objective of this study is to establish the time course over which viral amplification and clearance occurs after single and repeated administrations. We will test the working hypothesis that viral clearance will be delayed in CF33-NSC-treated mice as compared to mice receiving free CF33 since our preliminary data show loss of CF33 BLI signal in the free CF33 group after the 2^{nd} treatment. Viral presence within IP metastases at select time points will be determined using standard biochemical and immunological assays. Successful completion of this study will provide fundamental, necessary data demonstrating improved viral persistence and distribution when delivered using an NSC vehicle. The time course of viral persistence identified will inform the repeat dosing intervals used for determining the therapeutic efficacy and anti-cancer immune response of CF33-NSCs versus free CF33 in vivo.

Research Design: Immunocompetent syngeneic 8-12 week old female C57Bl/6 mice will be inoculated IP with ID8 tumor cells (Table 1). As per previous growth kinetics studies of this model, by 3 weeks after inoculation, mice will have established a few macroscopic and numerous (>20) microscopic tumors, ensuring an adequate number of metastatic tumors available for analysis. BLI will be used to confirm tumor engraftment. Experiment 2.1: Mice bearing engrafted tumors will be IP administered the predetermined dosage of CF33-NSCs or free CF33 (matched viral load). At select time points (pretreatment, and 1 h, 3 d, and 7 d after the last treatment) 10 mice will be euthanized to determine viral persistence within metastatic tumors. Experiment 2.2: Two CF33-NSC administrations will be delivered, spaced according to the interval determined in Experiment 1.2. For both experiments, the location, size, and weight of macroscopic tumors will be recorded. The viral load within 10 tumors per mouse will be quantitatively determined using both qPCR (for CF33 gene expression) and viral titration assays. The remaining tumors will be sectioned and stained with anti-vaccinia ab for CF33 to determine the time course of CF33 tumor penetration. BLI data will also be utilized to assess clearance and retention profile of the virus.

Biostatistical analysis: The primary outcome of these experiments is virus retention. since the slower the clearance, the higher the virus retention and the more time for virus to replicate in the tumor tissue. At each time point, virus retention will be compared between the free CF33 and CF33-NSC groups. Using a chi-square test with a 0.05 one-sided significance level, we will achieve statistical power of at least 85% to detect the difference between the virus retention, p1, for the CF33-NSCs (predicted to be 0.65) and p2 for the free CF33 group (predicted to be 0.10) when the sample size in each group is 10. Exploratory repeated measures analysis will be carried out to examine possible trends in retention over time. Multiple comparisons will not be corrected because of the relatively small sample size. Similar analyses will be carried out for clearance kinetics using BLI data.

Expected Outcomes: We expect to observe tumor tropism of CF33 NSCs within the first hour after injection. After a delay of 24 hours, the viral dose initially delivered to the tumors is expected to have amplified within the NSCs to induce lysis and transfer of mature viral particles to neighboring tumor cells. We expect to identify the time window during which viral load peaks and clears (1 week expected based on preliminary data). We also expect to see consistent viral clearance rates upon repeated treatment rounds when CF33 is delivered via NSCs, but accelerated clearance rates when delivered as a free virus. This information will be used for determining therapeutic efficacy and anti-cancer immune response of CF33 versus NSC-CF33 and will guide choice of dose interval frequency. Finally, we expect to see improved CF33 penetration and distribution in CF33-NSCs relative to free virus, given that NSCs will have 24 hours to penetrate biological barriers before they lyse. These barriers limit free viral distribution, which relies on passive viral diffusion. Success Criteria: Determination of the time required for 90% of initially delivered CF33 to clear from IP metastases. The longer the time required for this clearance the more effective we expect treatment to be.

Potential Pitfalls and Alternatives: In the unlikely event that both viral amplification and clearance are unable to be observed over the chosen timescale, time points will be adjusted to allow more/less time. Although we expect the viral load to be relatively consistent among the various metastases (given our observation that NSC tropism to ovarian metastases occurs irrespective of size or location) it is possible that some tumors will attract NSCs more than others or be better able to amplify the virus. Even if this is the case, a peak overall viral load should be identified within the tumors by averaging the load within multiple metastases.

Determine the Therapeutic Efficacy and Anti-Cancer Immune Response of CF33-NSCs Versus Free CF33 In Vivo (with and without Checkpoint Inhibitors).

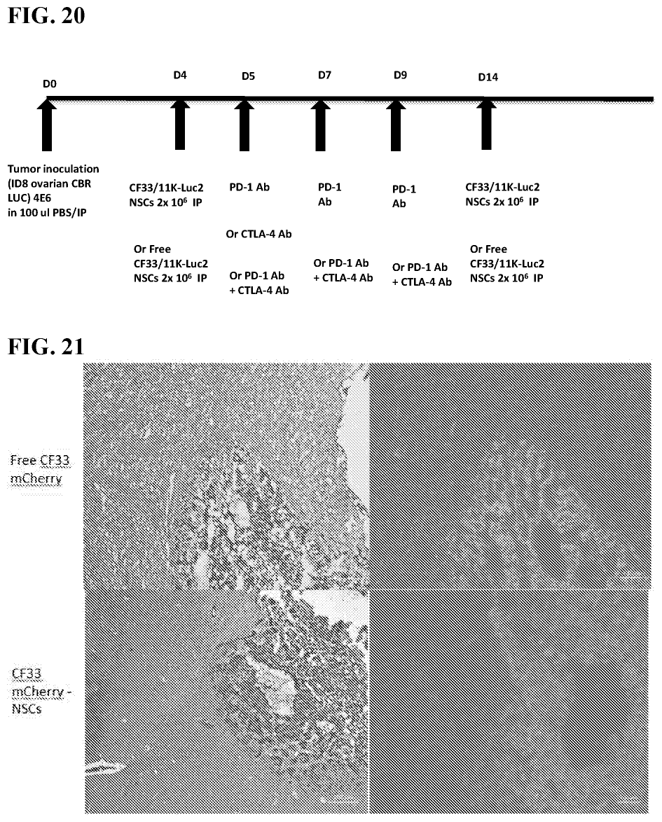

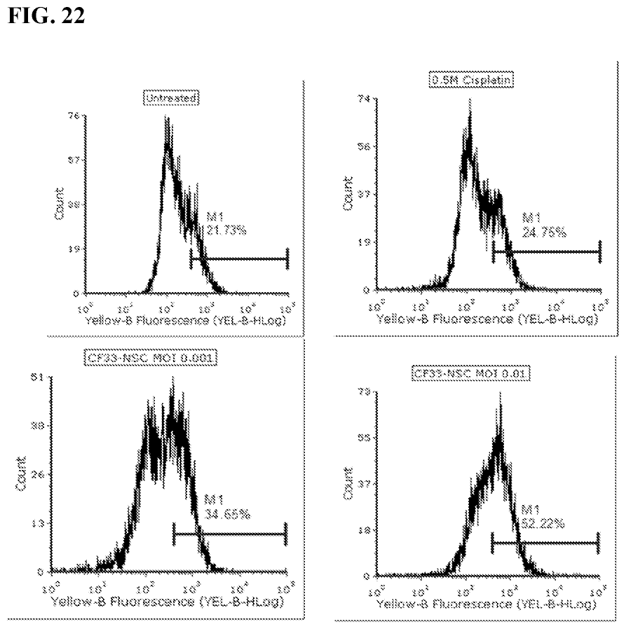

Introduction: We will assess the therapeutic efficacy of CF33-NSCs. The objective of this study is to establish pre-clinical data demonstrating whether CF33-NSC treatment is effective as a stand-alone therapy or if co-administration with a PD-L1 antibody (PD-L1 Ab) might confer additional therapeutic benefit, as it might overcome tumor immune tolerance and enhance immune recognition of cancer cells. We will test whether repeated rounds of CF33-NSC injections alone or with a PD-L1 antibody will significantly improve the survival of tumor-bearing mice as compared to control mice that receive no treatment or free CF33 with or without checkpoint inhibition (FIG. 20). We will use a standard Kaplan-Meier survival comparison to determine the utility of this treatment in immunocompetent syngeneic mice. Changes in the composition/activity of tumor-associated immune cells, such as dendritic cells, macrophages, myeloid-derived suppressor cells, NK, and T lymphocytes, as well as the expression of PD-L1 and PD1 on myeloid cells and T lymphocytes, respectively, will be evaluated using flow cytometry and functional assays. Successful completion of this Aim will contribute new information regarding the potential efficacy of CF33-NSCs for treating ovarian cancer and the extent to which the oncolytic and immune-stimulatory properties of CF33 are responsible for this effect.

Immune checkpoint blockade is inefficient in ‘cold’ tumors, which are poorly infiltrated by immune cells and also have low expression of PD-L1 on their surface. In the absence of available targets, immune checkpoint blockers like anti-PD-L1 (which targets PD-L1 expressed on the surface of cancer cells or on antigen-presenting cells), alone or in combination with anti-CTLA-4, remain therapeutically inefficient. Therapeutic administration of oncolytic viruses into tumors promotes the expression of PD-L1 on the surface of cancer cells and PD-1 and CTLA4 on immune cells. Thus, antiviral immunological events inflame the tumor, making it ‘hot’ and suitable for checkpoint blockade cancer immunotherapies. When checkpoint inhibitors are subsequently administered, they can bind to their respective targets on either cancer or immune cells. In addition, anti-PD-L1 therapy can stimulate an immune response to the tumor to strengthen the effects of the virus (FIG. 25). NK cell: Natural killer cell; PD-1: Programmed death-1; PD-L1: Programmed death ligand-1; CTLA-4: Cytotoxic T-lymphocyte-associated protein 4. Furthermore, oncolytic viruses can sensitize tumors to the therapeutic effects of immune checkpoint inhibition, including anti-PD-L1 therapy. Thus, combined oncolytic virus and checkpoint blockade therapy is shown to be highly effective against cancer (FIG. 26).

Exemplary immune checkpoint inhibitors used herein include without limitation, anti-PD-1, anti-PD-L2, anti-CTLA-4, Anti-TI<−3/LAG3, Anti-OX40, Anti-IDO, Anti-GITR, Anti-4-1BB/CD137]]

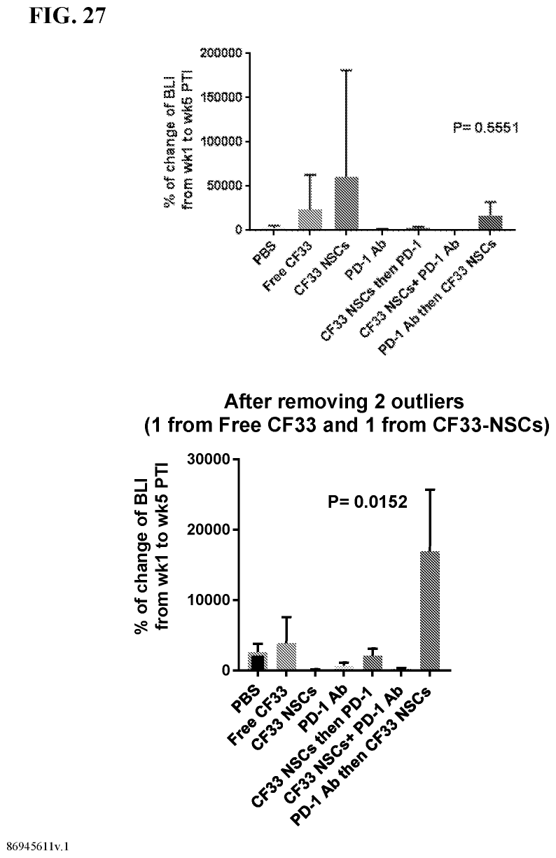

Research Design: Immunocompetent female C57Bl/6 mice (8-12 weeks old; n=80) will be inoculated IP with ID8 tumor cells. Treatment will begin one week later to mimic the clinical scenario of early stage or optimal post-surgical debulking of stage III ovarian cancer. Five treatment groups (16 mice/group) will include: 1) PBS (control); 2) free CF33; 3) CF33-NSCs; 4) CF33-NSCs+PD-L1 Ab (200 ug/mouse i.p.); and 5) PD-L1 Ab only. Groups 2-4 will have a matched viral load determined in Aims 1 and 2. Mice will be randomly assigned to each group. Four mice per group will be euthanized one week after two repeated treatment rounds. The remaining 12 mice will receive two additional treatment rounds and will be followed for long-term survival. Blood will be sampled weekly for assessment of CD4 and CD8 cells, regulatory T cells (Tregs), tumor associated macrophages (TAMs), dendritic cells and proinflammatory (IFNs, IL-1, IL-6, TNFa) vs immunosuppressive (IL-10, TGFb) cytokines, and anti-pox antibodies will be measured using Luminex assays. Whole body BLI will be performed as described above to follow tumor progression and viral distribution (FIG. 27). Coelenterazine IV (100 μl) and D-luciferin potassium salt (150 mg/kg) will be injected IP 10 min before in vivo imaging. After euthanasia or natural death, 10 tumors per mouse and peritoneal organs will be processed to determine viral load by viral titrations performed using dissociated tumor lysates. The metrics for each comparison are: 1) expression of the orthopoxvirus A-type inclusion protein (ATI) gene per mg of tumor and 2) viral titer/mg tumor and flow cytometry. Remaining tissues will be sectioned and stained to detect immune cell markers (including CD4 and CD8; dendritic cell cytokines Th1, Th2 and Th17; and anti-pox antibodies). Blood collected by cardiac puncture and peritoneal fluid aspirated by a syringe will be collected right after euthanasia and assayed by qPCR for these immune cells and viral particles. The immune and cancer cell viability/proliferation will be evaluated using flow cytometry and microscopy after Annexin V and/or BrdU staining. We will assess tumor infiltration by cytotoxic/exhausted CD8⁺ T cells (PD1⁺/CTLA4⁺/LAG3⁺), CD4⁺Foxp3⁺ Tregs, myeloid-derived suppressor cells (CD11b⁺Gr1⁺), and tumor associated macrophages (TAMs; CD11b⁺F4/80⁺). In TAMs and dendritic cells we will assess markers of APC activation, such as MHC class II, CD40, CD80, CD86, IL-12 secretion, and IFN. Cytokines analysis from tumor tissue will be assessed using cytokine microarrays. To verify whether any of the tested treatments generates immune responses against specific tumor antigens, we will rechallenge surviving mice with an unrelated orthotopic cancer model (e.g., MC38) after the initial ID8 challenge. Finally, we will use ID8-OVA cells to verify the generation of tumor antigen-specific T cell-specific immune responses using IFNγ ELISPOT assays after recall stimulation with MHC class I- (OVA₂₅₇₋₂₆₄) or II- (OVA₃₂₃₋₃₃₉) restricted peptides.

Biostatistical analysis: The primary outcome is long-term survival. In particular, we will compare survival proportions between the mice given CF33-NSCs (group 3) and those given free CF33 (group 2) at 120 days post final treatment. Using a chi-square test with a 0.05 one-sided significance level, we will achieve statistical power of at least 80% to detect a survival proportion difference of 0.50 (assuming survival proportions of 0.20 for the free CF33 group and 0.70 for the CF33-NSC group) when the sample size for each group is 12. Similar analyses will be conducted to compare CF33-NSC+PD-L1 Ab (group 4) to no treatment (group 1) and PD-L1 Ab alone (group 5) and CF33-NSC (group 3) treatments. Viral titer comparisons will be explored using linear mixed effects models to account for inter-mouse variability.

TABLE 1
Summary of in vivo studies

| | | |
|---|---|---|
| Study Proposed | Study population/Study groups | Sample size (SS) |

Determine the optimal IP CF33-NSC dose
for delivery of maximal viral load

| | | |
|---|---|---|
| 1.1. | ID8 Model | Total = 20 mice |
| | 2 Treatment groups of Dil labeled FFLUC CF33- | 2 × 10 mice/dose = 20 mice |
| | NSCs | |
| | 1) IP-0.1 × 10⁶ | |
| | 2) IP-2 × 10⁶ | |
| 1.2. | ID8 Model | Total = 20 mice |
| | 2 Treatment groups of Dil labeled FFLUC CF33- | 2 groups |
| | NSCs | 2 × 10 mice/group = 20 mice |
| | 1) IP-2 × 10⁶ | |
| | 2) 4 Tx IP-0.5 × 10⁶ | |

Determine the clearance kinetics of CF33 in vivo

| | | |
|---|---|---|
| 2.1 | ID8 Model | Total = 70 mice |
| | 1 dose Dil labeled FFLUC CF33-NSCs (IP-2 × 10⁶) | 2 studies × 4 groups per study |
| | or free FFLUC CF33 (IP- 3 × 10⁶ PFU, matched | (but with only one |
| | viral load) | pretreatment group since it is |
| | 4 groups per study: | the same for both) × 10 |
| | 1) pretreatment | mice/group |
| | 2) 1 hr after treatment | |
| | 3) 3 d after treatment | |
| | 4) 7 d after treatment | |
| 2.2 | ID8 Model | Total = 70 mice |
| | 2 doses Dil labeled FFLUC CF33-NSCs (IP-2 × 10⁶) | 2 studies × 4 groups per study |
| | or free FFLUC CF33 (IP- 3 × 10⁶ PFU, matched | (but with only one |
| | viral load) spaced according to the interval | pretreatment group since it is |
| | determined in Experiment 2.1 | the same for both) × 10 |
| | 4 groups per study: | mice/group |
| | 1) pretreatment | |
| | 2) 1 hr after 2nd treatment | |
| | 3) 3 d after 2nd treatment | |
| | 4) 7 d after 2nd treatment | |

Determine the therapeutic efficacy and anti-cancer
immune response of CF33-NSCs versus free CF33 in vivo

| | |
|---|---|
| ID8 Model | Total = 80 mice |
| Treatment groups will receive 2 Treatments of | 5 groups per study × 16 |
| the following based on interval determined in | mice/group |
| Experiment 2.2 starting 1 week after tumor | |
| implantation: | |
| 1) PBS (IP) (Control) | |
| 2) Free FFLUC CF33 (IP- 3 × 10⁶ PFU) | |
| 3) FFLUC CF33-NSCs (IP-2 × 10⁶) | |
| 4) αPD-L1 (IP-9″ mg/kg) | |
| 5) FFLUC CF33 -NSCs (IP-2 × 10⁶) + αPD-L1 (IP- | |
| 9″ mg/kg) | |
| 4 mice will be euthanized one week after the | |
| 2nd treatment and the remaining 12 will be left | |
| for long term survival | |

Scientific Rigor: To ensure scientific rigor, CF33-NSCs will be made from a research-grade bank throughout entire whole study. We will determine the optimal CF33-NSC administration protocol by testing several dosing regimens in while determining the optimal IP CF33-NSC dose for delivery of maximal viral load and determining the clearance kinetics of CF33 in vivo. Because the oncolytic activity of CF33-NSCs is based on the contribution of an active immune system, it is critical that we test it in a syngeneic immunocompetent mouse model. The only currently available model meeting these criteria is C57Bl/6 mice engrafted with ID8 cells. The C57Bl/6 mice will be purchased from Jackson Laboratories. Based on our earlier studies, we anticipate that 12 mice per group will be required to evaluate significance. CF33-NSCs were initially tested on human ovarian cancer OVCAR8 cells to show its permissiveness to CF33, thus supporting the feasibility of using CF33 in clinical trials. We have incorporated blinding and randomization to reduce bias, adhere to strict laboratory practices for data collection and analyses, and ensure transparency in reporting results. The order of treatment of individual mice will be randomized using a random number generator to avoid grouping identical protocols in time. Separate investigators will be responsible for assigning treatment protocols, performing treatments, and collecting data. For protocols involving data acquisition, the investigators will be blinded to treatment conditions and will not be made aware of the treatment allocations until all data have been collected and analyzed.

### Example 3

The following studies demonstrate the efficacy of NSC-CF33 and collectively compare antitumor activity of NSCs CF33 (with mCherry, EGFP, or Luciferase), oncolytic viruses CF17 or CF189 for brain (GL261 and U87), breast (4T1 and MCF-7) and ovarian (ID8 and OVCAR8) cancer cell lines. A ratio of 1 NSCs oncolytic virus:1000 cancer cells was used. CF33 is shown to be the most efficacious oncolytic chimeric pox virus followed by CF17 with CF189 the least potent virus.

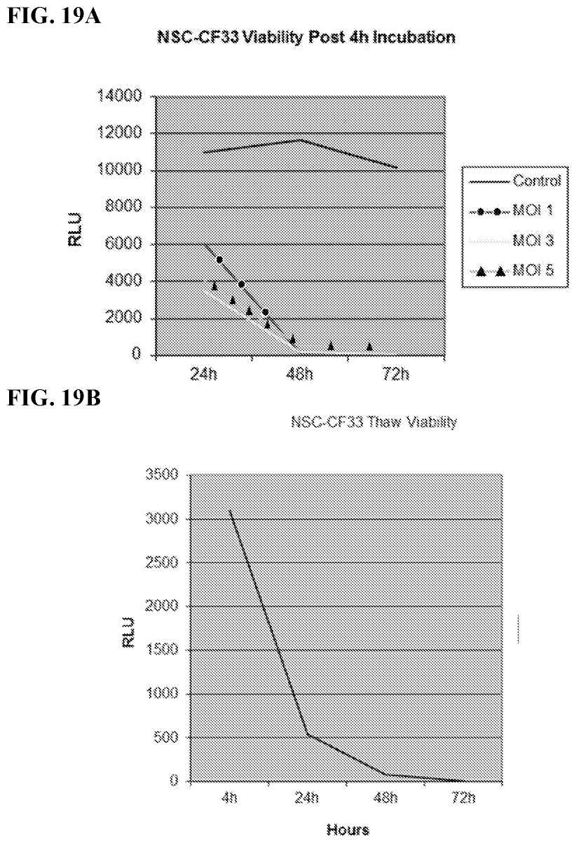

Neural stem cell (NSC) line HB1.F3.CD was tested for viability post incubation with CF33 and the CF33 loaded HB1.F3.CD was again assessed for viability post-thaw. HB1.F3.CD cells were incubated for 4 hours in transfection media with CF33 virus at MOI=3 and cell viability was tested (FIG. 19A). Cells were stored in Cryostor media and frozen. After thawing, the NSC-CF33 thaw viability was tested (FIG. 19B). The NSC-CF33 were then added to tumor cells at a ratio of 1 NSC-CF33:to 1000 cancer cells for in vitro experiments to test mouse and human cancer permissiveness to CF33 when CF33 is carried by NSCs.

Treatment combinations of free CF33/11K-Luc2 or CF33/11K-Luc2 carried by NSCs will be used for CF33-NSCs ovarian cancer studies. The following treatment combinations with two weekly treatments will be used in the studies (Table 2).

TABLE 2
Treatment Combinations

| | Number of animals × cells (NSCs) × |
|---|---|
| Composition administered | viral concentration |
| PBS | 8 mice × 6 PFU |
| PD-1 Ab | 8 mice × 6 PFU |
| CTLA1 (or GITR or ox40 or | 8 mice × 6 PFU |
| IDO etc.) | |
| PD-1 Ab + CTLA4Ab | 8 mice × 6 PFU |
| Free CF33 | 8 mice × 6 PFU |
| CF33-NSCs | 8 mice × (4 × 10⁶ cells) × 3 PFU |
| CF33-NSCS + PD-1 | 8 mice × (4 × 10⁶ cells) × 3 PFU |
| CF33-NCSs + CTLA4 | 8 mice × (4 × 10⁶ cells) × 3 PFU |
| CF33-NSCs + PD-1 + CTLA4 | 8 mice × (4 × 10⁶ cells) × 3 PFU |

The range of MOI for oncolytic viral infection into carrier cells is MOI=1 to 5. The optimized MOI is MOI=3. The dosage range for oncolytic virus-NCS administration is 100,000,000 to 200,000,000 cells. The optimized administration schedule is 150,000,000 cells once a week for two weeks. The clinical protocol for NSC administration in combination with virus is injection of 150,000,000 CF33-NSC once a week for two weeks simultaneously with ICI.

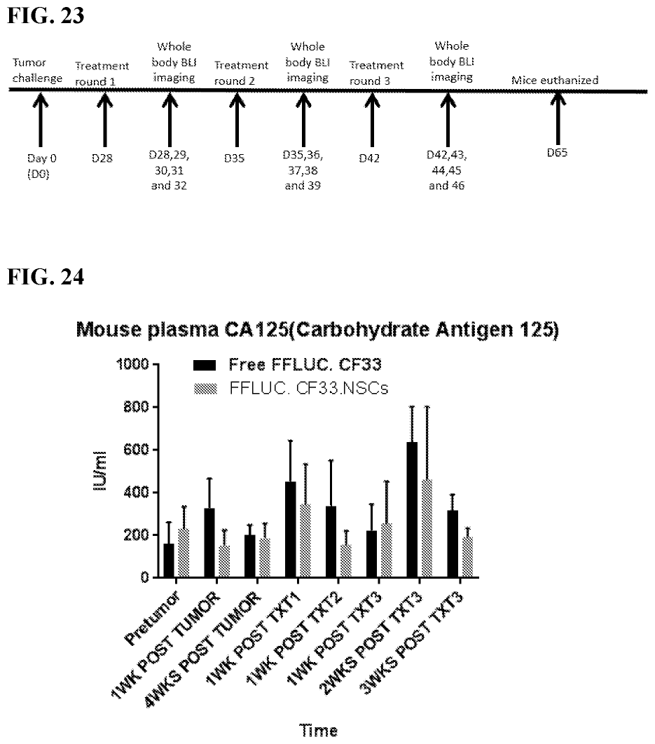

In vivo efficacy of the free CF33 virus versus the NCS-CF33 will be investigated in mouse models for human ovarian cancer (FIG. 23). On day 0, eight fourteen week old female C57BL/6 mice will be IP implanted with 10×10⁶ ID8 cells that express lentivirus-tdTOMATO (ID8 LV tdTOM). Three treatment rounds will be completed on days 28, 35, and 42. The treatment rounds include IP injection of 500 uL of 3.0×10⁶ PFU free CF33 per mouse in a four mice group, and IP injection of 500 uL of 2.0×10⁶ NSC-CF33 in second four mice group. Bioluminescent (BLI) imaging will be used to monitor tumor progression immediately after each treatment round and 1 day, 2 days, 3 days, and 4 days after each treatment round. Mice will be euthanized on day 65 to detect tumor progression. Harvesting will be conducted by collecting the pluck, diaphragm, and peritoneal fluid of the mice.

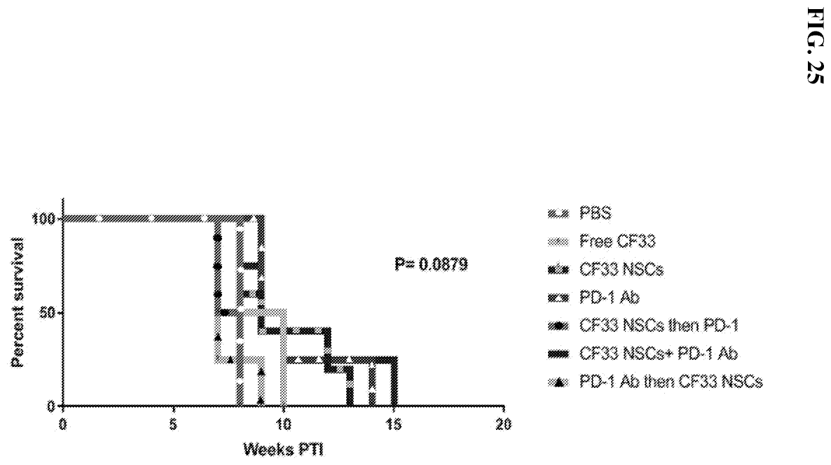

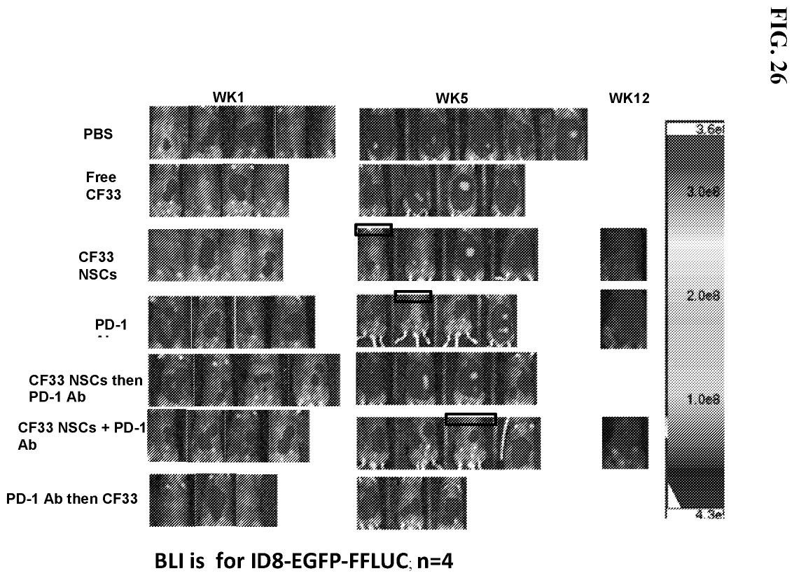

In vivo efficacy of free CF33 versus NCS-CF33 treated mice was assessed by CA 125 levels (cancer antigen 125). Levels of blood CA 125 are used to monitor certain cancers during and after treatment. CA 125 measurements were completed at intervals following tumor-implantation and treatment with either free FFLuc-CF33 or FFLuc-CF33-NSC. Mouse plasma CA 125 levels in mice treated with NSC-CF233 were consistently lower than in mice treated with free CF33, with the exception of one week following Treatment Round 3 (FIG. 24). Mouse plasma CA 125 levels decreased drastically from two weeks post-Treatment Round 3 to three weeks post-Treatment Round 3.

### Example 4: Thaw and Preparation of HB2.F3.CD_CRAD-S-PK7 for Clinical Administration

PURPOSE: To provide detailed instructions for the preparation of HB2.F3.CD_CRAd-S-pk7, Neural Stem Cell loaded with CRAd-S-pk7 adenovirus from a cryopreserved bank for clinical administration.

SCOPE: This procedure applies to the preparation of cells for clinical administration from a cryopreserved vial(s) of HB2.F3.CD_CRAd-S-pk7 Cell Bank. This procedure will be used for the preparation of HB2.F3.CD_CRAd-S-pk7 cells in the Cellular Therapies Production Center (CTPC) or other City of Hope (COH) Cleanroom Facilities for clinical administration. This is intended for use by the Principal Investigator for clinical trials conducted at City of Hope.

Procedure:

Procedural Set Up

Wipe down all working surfaces in the clean room with 70% IPA. Turn on the Biosafety Cabinet and turn on UV for a minimum of 15 minutes. Turn on the centrifuge to 4° C. and set it at 1500 rpm for 5 minutes.

Preparation of PFCNS+2% HSA (Wash Buffer/Product Diluent)

Remove packaging from the 125 mL sterile bottle and clearly label the bottle “PFCNS+2% HAS; initials, date, and time”. Remove outer packaging from the Perfusion Fluid-CNS (PFCNS) box. Using a wipe sprayed with IPA, carefully snap off top each of the 19 vials of PFCNS. Using a 10 mL syringe with needle, transfer 92 mL of PFCNS into the labeled bottle. Using a 10 mL syringe with needle, transfer 8 mL of 25% Human Serum Albumin (HSA) to the labeled bottle.

Thaw & Washing of the Cells

Affix the three labels (Label 1) obtained from QA on three 50 mL conical tubes that will be used to store the wash supernatants. Fill in the expiration date (60 days post preparation) and designation Wash #1, Wash #2, and Wash #3, respectively. Obtain a new 50 mL conical tube and label it as “A”. using a 50 mL serological pipette transfer 30 mL of wash buffer into this tube.

Thaw vials quickly until no ice is visible. Record the time of start and end time below. Immediately, wipe the vial with 70% IPA and place it in the Biologic Safety Cabinet (BSC). Centrifuge (4° C.) the “A” tube for 10 minutes at 1500 rpm. After centrifugation, carefully collect all supernatant into the tube labeled “Wash #1”. Be careful not to disturb the cell pellet. This supernatant will be used for Bactec/Fungal testing.

Resuspend the cell pellet in 40 mL of wash buffer for second wash. Centrifuge (4° C.) for 10 minutes at 1500 rpm. After centrifugation, carefully collect all supernatant into the tube labeled “Wash #2”. Be careful not to disturb the cell pellet.

Obtain a new 15 mL conical tube and label it as “B”. Resuspend the cell pellet in 5 mL of wash buffer and transfer the cells to the new tube labeled “B”. Rinse the “A” tube with an additional 5 mL wash buffer and transfer it into “B” tube. (10 mL total). This cell suspension (Tube B) will be used for the viability and gram stain release tests.

Viability and Cell Count of HB2.F3.CD_CRAd-S-Pk7 Cells:

Based on the reference table below (Table 3), take an aliquot form the tube “B” to count. Manually count viable and dead cells according to SOP-0306 Trypan Blue exclusion using Hemocytometer (minimum count 4 squares of 100 cells). Record values and complete calculations (Table 4).

TABLE 3
Suggested Dilution for Cell Count and Viability
Suggested Dilution for Cell Count and Viability by Trypan Blue Exclusion

| | | | | | |
|---|---|---|---|---|---|
| Cell | Approximate | | Cell | 0.04% Trypan | |
| Dose | Concentration of Cells in | Dilution | Suspension | Blue Volume | Check the |
| Level | suspension (cells/mL) | Factor | Volume (uL) | (uL) | row used |

| | | | | |
|---|---|---|---|---|
| 5.0E+07 | ~5.6E6 | 20 | 20 | 380 |
| 5.0E+08 | ~1.1E7 | 30 | 10 | 290 |
| 1.5E+08 | ~1.7E7 | 40 | 10 | 390 |

TABLE 4
Data Entry Table 1
Viability and Cell Count Calculations

Number of squares counted (A)
Number of viable cells (B)
Number of dead cells (C)
Total Number of cells (B + C) = D
Viability of cells (%) (B/D × 100
Viable Cell Concentration
(cells/mL),
E = (B/A) × Dilution factor × 10⁴
Total number of viable cells
E × 10 mL = F

Gram Stain of Cell Suspension in Wash

Obtain a sterile 2 mL cryovial and affix the Gram Stain label (Label 3). Transfer 400 uL of wash buffer into this vial. Transfer 20 uL from tube “B” to the vial labeled Gram Stain. Place sample in a sterile bag and place on ice.

Final Product Formulation

Centrifuge tube “B” (for the 3^{rd} wash) at 4° C. for 10 minutes at 1500 rpm. After centrifugation, carefully collect the supernatant into the tube labeled “Wash #3”. This supernatant will be used for Endotoxin testing.

Using a 2 mL pipette, transfer 2 mL of PFCNS+2% HSA (diluent) to gently resuspend the cell pellet. Draw up the entire content of the tube to measure the volume of cell suspension and record Volume of cell suspension measured (mL), G in Table 6. Complete calculations in Table 6 based on values calculated in Table 4. To achieve desired cell concentration for patient dose level (Table 5), add volume of diluent as calculated to tube B. Transfer entire product to the 4 mL cryovial.

TABLE 5

| Patient | | Cell |
|---|---|---|
| Dose | Clinical | Concentration |
| Level | Dose | (cells/mL) |
| 1 | 5.00E+07 | 1.67E+07 |
| 2 | 1.00E+08 | 3.33E+07 |
| | 1.50E+08 | 5.00E+07 |

TABLE 6
Data Entry Table 2
Data Entry Table 2

Total number of viable cells from Data Entry Table 1, F
Volume of cell suspension measured (mL), G
Cell Concentration of patient dose level, H
Final volume required to achieve cell concentration for
patient dose level (F/H) = I
Volume of diluent to be added I − G = J

Bactec and Fungal Sample

Obtain a sterile 4 mL vial and transfer 3 mL from the tube labeled Wash #1 to this tube. Place sample in sterile bag and place on ice. This sample will be used for product prep for STAT testing.

Endotoxin Determination

Obtain a sterile 2 mL cryovial and label Endotoxin. Transfer 450 uL of wash buffer into this vial. Transfer 50 uL from Wash #3 to the vial labled Endotoxin. Place sample in a sterile bag and place on ice. This sample will be used for product prep for STAT testing.

### REFERENCES

1. Jemal, A., et al., Cancer statistics, 2008. CA Cancer J Clin, 2008. 58(2): p. 71-96.
2. Li, S., et al., Oncolytic virotherapy for ovarian cancer. Oncolytic Virother, 2012. 1: p. 1-21.
3. Cannistra, S. A., Cancer of the Ovary. New England Journal of Medicine, 2004. 351(24): p. 2519-2529.
4. Kim, J., et al., Stem Cell-Based Cell Carrier for Targeted Oncolytic Virotherapy: Translational Opportunity and Open Questions. Viruses, 2015. 7(12): p. 6200-17.
5. Ding, J., Oncolytic virus as a cancer stem cell killer: progress and challenges. Stem Cell Investigation, 2014. 1(12).
6. Mader, E. K., et al., Mesenchymal stem cell carriers protect oncolytic measles viruses from antibody neutralization in an orthotopic ovarian cancer therapy model. Clin Cancer Res, 2009. 15(23): p. 7246-55.
7. Hartkopf, A. D., et al., Oncolytic Viruses to Treat Ovarian Cancer Patients—a Review of Results From Clinical Trials. Geburtshilfe Frauenheilkd, 2012. 72(2): p. 132-136.
8. Ahmed, A. U., et al., A preclinical evaluation of neural stem cell-based cell carrier for targeted antiglioma oncolytic virotherapy. J Natl Cancer Inst, 2013. 105(13): p. 968-77.
9. Heise, C., et al., Efficacy of a replication-selective adenovirus against ovarian carcinomatosis is dependent on tumor burden, viral replication and p53 status. Gene Ther, 2000. 7(22): p. 1925-9.
10. Lv, Y.-G., et al., The role of survivin in diagnosis, prognosis and treatment of breast cancer. Journal of Thoracic Disease, 2010. 2(2): p. 100-110.
11. Falleni, M., et al., Survivin gene expression in early-stage non-small cell lung cancer. J Pathol, 2003. 200(5): p. 620-6.
12. Lv, S., et al., The Impact of Survivin on Prognosis and Clinicopathology of Glioma Patients: A Systematic Meta-Analysis. Molecular Neurobiology, 2015. 51(3): p. 1462-1467.
13. Armstrong, D. K., et al., Intraperitoneal Cisplatin and Paclitaxel in Ovarian Cancer. New England Journal of Medicine, 2006. 354(1): p. 34-43.
14. Sun, V., et al., Toxicities, complications, and clinical encounters during intraperitoneal chemotherapy in 17 women with ovarian cancer. Eur J Oncol Nurs, 2013. 17(3): p. 375-80.
15. Roulstone, V., et al., Phase I Trial of Cyclophosphamide as an Immune Modulator for Optimizing Oncolytic Reovirus Delivery to Solid Tumors. Clinical cancer research: an official journal of the American Association for Cancer Research, 2015. 21(6): p. 1305-1312.
16. Bucheit, A. D., et al., An oncolytic measles virus engineered to enter cells through the CD20 antigen. Mol Ther, 2003. 7(1): p. 62-72.
17. Eto, Y., et al., Development of PEGylated adenovirus vector with targeting ligand. Int J Pharm, 2008. 354(1-2): p. 3-8.
18. Cole, C., et al., Tumor-targeted, systemic delivery of therapeutic viral vectors using hitchhiking on antigen-specific T cells. Nat Med, 2005. 11(10): p. 1073-81.
19. Power, A. T. and J. C. Bell, Taming the Trojan horse: optimizing dynamic carrier cell/oncolytic virus systems for cancer biotherapy. Gene Ther, 2008. 15(10): p. 772-779.
20. Aboody, K. S., et al., Neural stem cell-mediated enzyme/prodrug therapy for glioma: preclinical studies. Sci Transl Med, 2013. 5(184): p. 184ra59.
21. Otsuka, S. and Bebb, G., The CXCR4/SDF-1 chemokine receptor axis: a new target therapeutic for non-small cell lung carcinoma. J Thorac Oncol., 2008 December; 3(12):1379-83
22. Bagó, J. R., Alfonso-Pecchio, A., Okolie, O., Dumitru, R., Rinkenbaugh, A., Baldwin, A. S., Miller, C. R., Magness, S. T., Hingtgen, S. D., Therapeutically engineered induced neural stem cells are tumour-homing and inhibit progression of glioblastoma. Nat Commun. 2016 Feb. 2; 7:10593
23. Mansfield, D. C., et al., Oncolytic vaccinia virus as a vector for therapeutic sodium iodide symporter gene therapy in prostate cancer. Gene Ther, 2016. 23(4): p. 357-68.
24. Parato, K. A., et al., The oncolytic poxvirus JX-594 selectively replicates in and destroys cancer cells driven by genetic pathways commonly activated in cancers. Mol Ther, 2012. 20(4): p. 749-58.
25. Matveeva, O. V., et al., Oncolysis by paramyxoviruses: multiple mechanisms contribute to therapeutic efficiency. Mol Ther Oncolytics, 2015. 2.
26. Liu, Z., et al., Rational combination of oncolytic vaccinia virus and PD-L1 blockade works synergistically to enhance therapeutic efficacy. Nat Commun, 2017. 8: p. 14754.
27. Hoff, C. M. a. M., Peter J., Technological Advances in Peritoneal Dialysis Research. International Society for Peritoneal Dialysis, 2006. 26: p. 547-558.
28. Hoff, C. M. and T. R. Shockley, Peritoneal dialysis in the 21st century: the potential of gene therapy. J Am Soc Nephrol, 2002. 13 Suppl 1: p. S117-24.
29. Mader, E. K., et al., Mesenchymal stem cell carriers protect oncolytic measles viruses from antibody neutralization in an orthotopic ovarian cancer therapy model. Clinical cancer research, 2009. 15(23): p. 7246-7255.
30. Mader, E. K., et al., Optimizing patient derived mesenchymal stem cells as virus carriers for a phase I clinical trial in ovarian cancer. J Transl Med, 2013. 11: p. 20.
31. Power, A. T., et al., Carrier cell-based delivery of an oncolytic virus circumvents antiviral immunity. Mol Ther, 2007. 15(1): p. 123-30.
32. Portnow, J., et al., Neural Stem Cell-Based Anticancer Gene Therapy: A First-in-Human Study in Recurrent High-Grade Glioma Patients. Clin Cancer Res, 2017. 23(12): p. 2951-2960.
33. Morshed, R. A., et al. Analysis of glioblastoma tumor coverage by oncolytic virus-loaded neural stem cells using MRI-based tracking and histological reconstruction. Cancer Gene Ther. 2015 January; 22(1):55-61.
34. Metz, M. Z., et al., Neural stem cell-mediated delivery of irinotecan-activating carboxylesterases to glioma: implications for clinical use. Stem Cells Transl Med, 2013. 2(12): p. 983-92.
35. Cao, P., et al., Intraperitoneal Administration of Neural Stem Cell-Nanoparticle Conjugates Targets Chemotherapy to Ovarian Tumors. Bioconjug Chem, 2017. 28(6): p. 1767-1776.
36. Wang, J., et al., Thymidine kinase 1 expression in ovarian serous adenocarcinoma is superior to Ki-67: A new prognostic biomarker. Tumour Biol, 2017. 39(6): p. 1010428317706479.
37. Aboody, K. S., et al., Neural stem cells display extensive tropism for pathology in adult brain: evidence from intracranial gliomas. Proc Natl Acad Sci USA, 2000. 97(23): p. 12846-51.
38. Brown, A. B., et al., Intravascular delivery of neural stem cell lines to target intracranial and extracranial tumors of neural and non-neural origin. Hum Gene Ther, 2003. 14(18): p. 1777-85.
39. Zhao, D., et al., Human neural stem cell tropism to metastatic breast cancer. Stem Cells, 2012. 30(2): p. 314-25.
40. Danks, M. K., et al., Tumor-targeted enzyme/prodrug therapy mediates long-term disease-free survival of mice bearing disseminated neuroblastoma. Cancer Res, 2007. 67(1): p. 22-5.
41. Ruf, B. and U. M. Lauer, Assessment of current virotherapeutic application schemes: “hit hard and early” versus “killing softly”? Molecular Therapy Oncolytics, 2015. 2: p. 15018.
42. Prestwich, R. J., et al., Oncolytic Viruses: Do They Have a Role in Anti-Cancer Therapy? Clinical Medicine. Oncology, 2008. 2: p. 83-96.
43. Aghi, M. and R. L. Martuza, Oncolytic viral therapies—the clinical experience. Oncogene, 2005. 24(52): p. 7802-16.
44. Laudanski, K., et al., Potential Pitfalls of the Humanized Mice in Modeling Sepsis. Int J Inflam, 2018. 2018: p. 6563454.
45. Dawidczyk, C. M., et al., State-of-the-art in design rules for drug delivery platforms: lessons learned from FDA-approved nanomedicines. J Control Release, 2014. 187: p. 133-44.
46. Rink, J. S., et al., Update on current and potential nanoparticle cancer therapies. Curr Opin Oncol, 2013. 25(6): p. 646-51.
47. Pattni, B. S., V. V. Chupin, and V. P. Torchilin, New Developments in Liposomal Drug Delivery. Chem Rev, 2015. 115(19): p. 10938-66.
48. Allen, T. M. and P. R. Cullis, Liposomal drug delivery systems: from concept to clinical applications. Adv Drug Deliv Rev, 2013. 65(1): p. 36-48.
49. Elsabahy, M. and K. L. Wooley, Design of polymeric nanoparticles for biomedical delivery applications. Chem Soc Rev, 2012. 41(7): p. 2545-61.
50. Wang, A. Z., R. Langer, and O. C. Farokhzad, Nanoparticle delivery of cancer drugs. Annu Rev Med, 2012. 63: p. 185-98.
51. Hong, R. L., et al., Direct comparison of liposomal doxorubicin with or without polyethylene glycol coating in C-26 tumor-bearing mice: is surface coating with polyethylene glycol beneficial? Clin Cancer Res, 1999. 5(11): p. 3645-52.
52. Armstrong, J. K., et al., Antibody against poly(ethylene glycol) adversely affects PEG-asparaginase therapy in acute lymphoblastic leukemia patients. Cancer, 2007. 110(1): p. 103-11.
53. Vabbilisetty, P. and X. L. Sun, Liposome surface functionalization based on different anchoring lipids via Staudinger ligation. Org Biomol Chem, 2014. 12(8): p. 1237-44.
54. Ruf, B. and U. M. Lauer, Assessment of current virotherapeutic application schemes: “hit hard and early” versus “killing softly”? Molecular Therapy—Oncolytics, 2015. 2: p. 15018.
55. Parato, K. A., et al., Recent progress in the battle between oncolytic viruses and tumours. Nat Rev Cancer, 2005. 5(12): p. 965-76.
56. Tsai, H. F., et al., Candida glabrata erg1 mutant with increased sensitivity to azoles and to low oxygen tension. Antimicrob Agents Chemother, 2004. 48(7): p. 2483-9.
57. Tsai, F. C., et al., A polarized Ca2+, diacylglycerol and STIM1 signalling system regulates directed cell migration. Nat Cell Biol, 2014. 16(2): p. 133-44.
58. Chen, Y., et al., Pre-existent adenovirus antibody inhibits systemic toxicity and antitumor activity of CN706 in the nude mouse LNCaP xenograft model: implications and proposals for human therapy. Hum Gene Ther, 2000. 11(11): p. 1553-67.
59. Casey, S. C., Tong, L., Li, Y., Do, R., Walz, S., Fitzgerald, K. N., Gouw, A., Baylot, V., Guetegemann, I., Eilers, M., & Felsher, D. W. (2016). MYC regulates the antitumor immune response through CD47 and PD-L1. Science.
60. Duraiswamy, J., Kaluza, K. M., Freeman, G. J., & Coukos, G. (2013). Dual Blockade of PD-1 and CTLA-4 Combined with Tumor Vaccine Effectively Restores T Cell Rejection Function in Tumors. Cancer Res, 73(12), 3591-603.
61. Gaillard, S. L., Secord, A. A., & Monk, B. (2016). The role of immune checkpoint inhibition in the treatment of ovarian cancer. Gynecologic Oncology Research and Practice, 3, 11.
62. Kong, Y.-c.M., & Flynn, J. C. (2014). Opportunistic Autoimmune Disorders Potentiated by Immune-Checkpoint Inhibitors Anti-CTLA-4 and Anti-PD-1. Frontiers in Immunology, 5, 206.
63. Willingham, S. B., Volkmer, J.-P., Gentles, A. J., Sahoo, D., Dalerba, P., Mitra, S. S., Wang, J., Contreras-Trujillo, H., Martin, R., Cohen, J. D., Lovelace, P., Scheeren, F. A., Chao, M. P., Weiskopf, K., Tang, C., Volkmer, A. K., Naik, T. J., Storm, T. A., Mosley, A. R., Edris, B., Schmid, S. M., Sun, C. K., Chua, M.-S., Murillo, O., Rajendran, P., Cha, A. C., Chin, R. K., Kim, D., Adorno, M., Raveh, T., Tseng, D., Jaiswal, S., Enger, P. O., Steinberg, G. K., Li, G., So, S. K., Majeti, R., Harsh, G. R., van de Rijn, M., Teng, N. N. H., Sunwoo, J. B., Alizadeh, A. A., Clarke, M. F., & Weissman, I. L. (2012). The CD47-signal regulatory protein alpha (SIRPa) interaction is a therapeutic target for human solid tumors. Proc Natl Acad Sci USA, 109(17), 6662-7.
64. Chen, L., & Han, X. (2015). Anti-PD-1/PD-L1 therapy of human cancer: past, present, and future. J Clin Invest, 125(9), 3384-91.
65. Sah, N. K., Khan, Z., Khan, G. J., & Bisen, P. S. (2006). Structural, functional and therapeutic biology of survivin. Cancer Lett, 244(2), 164-71.
66. Liguang, Z., Peishu, L., Hongluan, M., Hong, J., Rong, W., Wachtel, M. S., & Frezza, E. E. (2007). Survivin expression in ovarian cancer. Exp Oncol, 29(2), 121-5.
67. Pesonen, S., Kangasniemi, L., & Hemminki, A. (2011). Oncolytic adenoviruses for the treatment of human cancer: focus on translational and clinical data. Mol Pharm, 8(1), 12-28.
68. Shinoura, N., Yoshida, Y., Tsunoda, R., Ohashi, M., Zhang, W., Asai, A., Kirino, T., & Hamada, H. (1999). Highly augmented cytopathic effect of a fiber-mutant E1B-defective adenovirus for gene therapy of gliomas. Cancer Res, 59(14), 3411-6.
69. Vasey, P. A., Shulman, L. N., Campos, S., Davis, J., Gore, M., Johnston, S., Kirn, D. H., O'Neill, V., Siddiqui, N., Seiden, M. V., & Kaye, S. B. (2002). Phase I trial of intraperitoneal injection of the E1B-55-kd-gene-deleted adenovirus ONYX-015 (d11520) given on days 1 through 5 every 3 weeks in patients with recurrent/refractory epithelial ovarian cancer. J Clin Oncol, 20(6), 1562-9.
70. Douglas, J. T., Kim, M., Sumerel, L. A., Carey, D. E., & Curiel, D. T. (2001). Efficient oncolysis by a replicating adenovirus (ad) in vivo is critically dependent on tumor expression of primary ad receptors. Cancer Res, 61(3), 813-7.
71. Ulasov, I. V., Zhu, Z. B., Tyler, M. A., Han, Y., Rivera, A. A., Khramtsov, A., Curiel, D. T., & Lesniak, M. S. (2007). Survivin-driven and fiber-modified oncolytic adenovirus exhibits potent antitumor activity in established intracranial glioma. Hum Gene Ther, 18(7), 589-602.
72. Mittal, A. K., Chaturvedi, N. K., Rohlfsen, R. A., Gupta, P., Joshi, A. D., Hegde, G. V., Bociek, R. G., & Joshi, S. S. (2013). Role of CTLA4 in the Proliferation and Survival of Chronic Lymphocytic Leukemia. PLoS One, 8(8), e70352.
73. Okazaki, T., & Honjo, T. (2007). PD-1 and PD-1 ligands: from discovery to clinical application. Int Immunol, 19(7), 813-24.
74. Zitvogel, L., & Kroemer, G. (2012). Targeting PD-1/PD-L1 interactions for cancer immunotherapy. Oncoimmunology, 1(8), 1223-5.
75. Ansari, M. J. I., Salama, A. D., Chitnis, T., Smith, R. N., Yagita, H., Akiba, H., Yamazaki, T., Azuma, M., Iwai, H., Khoury, S. J., Auchincloss, H., & Sayegh, M. H. (2003). The Programmed Death-1 (PD-1) Pathway Regulates Autoimmune Diabetes in Nonobese Diabetic (NOD) Mice. J Exp Med, 198(1), 63-9.
76. Joshi, M. N., Whitelaw, B. C., Palomar, M. T., Wu, Y., & Carroll, P. V. (2016). Immune checkpoint inhibitor-related hypophysitis and endocrine dysfunction: clinical review. Clin Endocrinol (Oxf), 85(3), 331-9.
77. Nayerossadat, N., Maedeh, T., & Ali, P. A. (2012). Viral and nonviral delivery systems for gene delivery. Advanced Biomedical Research, 1, 27.
78. Nwanegbo, E., Vardas, E., Gao, W., Whittle, H., Sun, H., Rowe, D., Robbins, P. D., & Gambotto, A. (2004). Prevalence of neutralizing antibodies to adenoviral serotypes 5 and 35 in the adult populations of The Gambia, South Africa, and the United States. Clin Diagn Lab Immunol, 11(2), 351-7.
79. Mader, E. K., Butler, G., Dowdy, S. C., Mariani, A., Knutson, K. L., Federspiel, M. J., Russell, S. J., Galanis, E., Dietz, A. B., & Peng, K. W. (2013). Optimizing patient derived mesenchymal stem cells as virus carriers for a phase I clinical trial in ovarian cancer. J Transl Med, 11, 20.
80. Mickelson, E. M., Longton, G., Anasetti, C., Petersdorf, E., Martin, P., Guthrie, L. A., & Hansen, J. A. (1996). Evaluation of the mixed lymphocyte culture (MLC) assay as a method for selecting unrelated donors for marrow transplantation. Tissue Antigens, 47(1), 27-36.
81. Power, A. T., Wang, J., Falls, T. J., Paterson, J. M., Parato, K. A., Lichty, B. D., Stojdl, D. F., Forsyth, P. A., Atkins, H., & Bell, J. C. (2007). Carrier cell-based delivery of an oncolytic virus circumvents antiviral immunity. Mol Ther, 15(1), 123-30.
82. Zhu, Z. B., Makhija, S. K., Lu, B., Wang, M., Kaliberova, L., Liu, B., Rivera, A. A., Nettelbeck, D. M., Mahasreshti, P. J., Leath, C. A., Barker, S., Yamaoto, M., Li, F., Alvarez, R. D., & Curiel, D. T. (2004). Transcriptional targeting of tumors with a novel tumor-specific survivin promoter. Cancer Gene Ther, 11(4), 256-62.
83. Minev, B., Kohrt, H., Kilinc, M., Chen, N., Feng, A., Pessian, M., Geissinger, U., Haefner, E., Tsoneva, D., Bozhilov, K., Sagiv-Barfi, I., Zhao, X., Rajesekaran, N., Levy, R., & Szalay, A. (2014). Combination immunotherapy with oncolytic vaccinia virus and checkpoint inhibitor following local tumor irradiation. Journal for Immunotherapy of Cancer, 2(Suppl 3), P112-P.
84. Zamarin, D., Holmgaard, R. B., Subudhi, S. K., Park, J. S., Mansour, M., Palese, P., Merghoub, T., Wolchok, J. D., & Allison, J. P. (2014). Localized oncolytic virotherapy overcomes systemic tumor resistance to immune checkpoint blockade immunotherapy. Science translational medicine, 6(226), 226ra32-ra32.
85. Leng, S. X., McElhaney, J. E., Walston, J. D., Xie, D., Fedarko, N. S., & Kuchel, G. A. (2008). ELISA AND MULTIPLEX TECHNOLOGIES FOR CYTOKINE MEASUREMENT IN INFLAMMATION AND AGING RESEARCH. The journals of gerontology Series A, Biological sciences and medical sciences, 63(8), 879-84.
86. He, T.-C., Zhou, S., da Costa, L. T., Yu, J., Kinzler, K. W., & Vogelstein, B. (1998). A simplified system for generating recombinant adenoviruses. Proc Natl Acad Sci USA, 95(5), 2509-14.
87. Mizugaki, H., Yamamoto, N., Murakami, H., Kenmotsu, H., Fujiwara, Y., Ishida, Y., Kawakami, T., & Takahashi, T. (2016). Phase I dose-finding study of monotherapy with atezolizumab, an engineered immunoglobulin monoclonal antibody targeting PD-L1, in Japanese patients with advanced solid tumors. Invest New Drugs, 34(5), 596-603.
88. Kojima, Y., Volkmer, J.-P., McKenna, K., Civelek, M., Lusis, A. J., Miller, C. L., Direnzo, D., Nanda, V., Ye, J., Connolly, A. J., Schadt, E. E., Quertermous, T., Betancur, P., Maegdefessel, L., Matic, L. P., Hedin, U., Weissman, I. L., & Leeper, N. J. (2016). CD47-blocking antibodies restore phagocytosis and prevent atherosclerosis. Nature, 536(7614), 86-90.
89. Hulin-Curtis, S. L., Uusi-Kerttula, H., Jones, R., Hanna, L., Chester, J. D., & Parker, A. L. (2016). Evaluation of CD46 re-targeted adenoviral vectors for clinical ovarian cancer intraperitoneal therapy. Cancer Gene Ther, 23(7), 229-34.
90. Cao et al., Intraperitoneal Administration of Neural Stem Cell—Nanoparticle Conjugates Targets Chemotherapy to Ovarian Tumors, Bioconjugate Chem., 2017, 28 (6), pp 1767-1776
91. Mansfield et al., Oncolytic vaccinia virus as a vector for therapeutic sodium iodide symporter gene therapy in prostate cancer, Gene Therapy volume23, pages357-368 (2016)
92. Ahmed, A. U., et al., Neural stem cell-based cell carriers enhance therapeutic efficacy of an oncolytic adenovirus in an orthotopic mouse model of human glioblastoma. Mol Ther, 2011.19(9): p. 1714-26.
93. Wang et al., Enhancing Expression of Functional Human Sodium Iodide Symporter and Somatostatin Receptor in Recombinant Oncolytic Vaccinia Virus for In Vivo Imaging of Tumors, J Nucl Med Feb. 1, 2017 vol. 58 no. 2 221-227.
94. Haddad, et al., Genetically Engineered Vaccinia Viruses As Agents for Cancer Treatment, Imaging, and Transgene Delivery, Front. Oncol., 23 May 2017.
95. Li Z., Oganesyan D., Mooney R., Rong X., Christensen M. J., Shahmanyan D., Perrigue P. M., Benetatos J., Tsaturyan L., Aramburo S., Annala A. J., Lu Y., Najbauer J., Wu X., Barish M. E., Brody D. L., Aboody K. S., Gutova M. L-MYC expression maintains self-renewal and prolongs multipotency of primary human neural stem cells. Stem Cell Reports 7:483-495(2016).
96. Mooney, R., Majid, A. A., Batalla-Covello, J., Machado, D., Lui, X., Gonzaga, J., Tirughana, R., Hammad, M, Dellinger, T. H., Lesniak, M. S., Curiel, D. T. Aboody, K. S. Enhanced Delivery of Oncolytic Adenovirus by Neural Stem Cells for Treatment of Metastatic Ovarian Cancer. Mol Ther Oncolytics 12:79-92(2019).

### P1 EMBODIMENTS

### P1 Embodiment 1

A method of treating cancer, including administrating to a subject a therapeutically effective amount of neural stem cells (NSCs) and a replication-competent oncolytic virus.

### P1 Embodiment 2

The method of P1 embodiment 1, wherein virus is conditionally replication-competent chimeric orthopoxvirus CF33.

### P1 Embodiment 3

The method of P1 embodiment 1, further comprising administering anti-PD-L1, CTLA4 or OX40.

### P1 Embodiment 4

The method of P1 embodiment 1, wherein the cancer is peritoneal cancer selected from the group consisting of ovarian cancer, pancreatic cancer, colorectal cancer, gastric cancer, and liver cancer.

### P1 Embodiment 5

The method of P1 embodiment 1, wherein the cancer is metastatic ovarian cancer.

### P1 Embodiment 6

The method of P1 embodiment 1, wherein the NSCs are

HB1.F3.CD21.

### P1 Embodiment 7

The method of P1 embodiment 1, wherein the administration is by injection.

### P1 Embodiment 8

The method of P1 embodiment 1, wherein the administration is by intraperitoneal (IP) injection.

### P1 Embodiment 9

A pharmaceutical composition for treating cancer comprising a therapeutically effective amount of neural stem cells (NSCs) and a replication-competent oncolytic virus.

### P1 Embodiment 10

The pharmaceutical composition of P1 embodiment 9, wherein virus is conditionally replication-competent chimeric orthopoxvirus CF33.

### P1 Embodiment 11

The pharmaceutical composition of P1 embodiment 9, further comprising anti-PD-L1, CTLA4 or OX40.

### P1 Embodiment 12

The pharmaceutical composition of P1 embodiment 9, wherein the cancer is peritoneal cancer selected from the group consisting of ovarian cancer, pancreatic cancer, colorectal cancer, gastric cancer, and liver cancer.

### P1 Embodiment 13

The pharmaceutical composition of P1 embodiment 9, wherein the cancer is metastatic ovarian cancer

### P1 Embodiment 14

The pharmaceutical composition of P1 embodiment 9, wherein the NSCs are HB1.F3.CD21

### P2 EMBODIMENTS

### P2 Embodiment 1

A method of treating cancer, said method comprising administering to a subject in need thereof an effective amount of a chimeric poxvirus and a neural stem cell (NSC).

### P2 Embodiment 2

The method of P2 embodiment 1, wherein said chimeric poxvirus forms part of said NSC.

### P2 Embodiment 3

The method of P2 embodiment 2, wherein said NSC is administered intraperitonally.

### P2 Embodiment 4

The method of P2 embodiment 2, wherein said NSC is administered at a cell number of 1×10⁸ to 2×10⁸ cells.

### P2 Embodiment 5

The method of P2 embodiment 2, wherein said NSC is administered at a cell number of 1.5×10⁸ cells.

### P2 Embodiment 6

The method of claim 2, wherein said NSC is administered at a cell number of 5×10⁶ to 5×10⁷ cells/mL.

### P2 Embodiment 7

The method of any one of P2 embodiments 1 to 6, wherein said poxvirus is encoded by a nucleic acid sequence having a sequence identity of at least 70% to SEQ ID NO:1.

### P2 Embodiment 8

The method of P2 embodiment 7, wherein said nucleic acid sequence comprises nucleic acid fragments from at least two poxvirus strains selected from the group consisting of cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, vaccinia virus strain AS, orf virus strain NZ2 and pseudocowpox virus strain TJS.

### P2 Embodiment 9

The method of P2 embodiment 7 or 8, wherein said nucleic acid sequence comprises nucleic acid fragments from cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic and vaccinia virus strain AS.

### P2 Embodiment 10

The method of one of P2 embodiments 7-9, wherein said nucleic acid sequence further comprises one or more anti-cancer nucleic acid sequences or a detectable moiety-encoding nucleic acid sequence.

### P2 Embodiment 11

The method of one of P2 embodiments 1-10, wherein said chimeric poxvirus is a replication-competent chimeric poxvirus.

### P2 Embodiment 12

The method of one of P2 embodiments 1-11, wherein said NSC is a Human Leukocyte Antigen (HLA) II-negative NSC.

### P2 Embodiment 13

The method of one of P2 embodiments 1-12, wherein said NSC is an HB1.F3.CD21 cell.

### P2 Embodiment 14

The method of one of P2 embodiments 1-13, the method comprising further administering an effective amount of a therapeutic agent.

### P2 Embodiment 15

The method of P2 embodiment 14, wherein said therapeutic agent is an anti-cancer agent.

### P2 Embodiment 16

The method of P2 embodiment 14 or 15, wherein said therapeutic agent is selected from the group consisting of a small molecule, a nucleic acid, a polypeptide and an antibody.

### P2 Embodiment 17

The method of one of P2 embodiments 14-16, wherein said therapeutic agent is a PD-L1 inhibitor, a CTLA-4 inhibitor or an OX40 inhibitor.

### P2 Embodiment 18

The method of one of P2 embodiments 1-17, wherein said cancer is breast cancer, colon cancer, kidney cancer, leukemia, lung cancer, melanoma, ovarian cancer, prostate cancer, pancreatic cancer, brain cancer, liver cancer, gastric cancer or a sarcoma.

### P2 Embodiment 19

The method of P2 embodiment 18, wherein said ovarian cancer is an ovarian metastasis.

### P2 Embodiment 20

A composition comprising a chimeric poxvirus and a neural stem cell (NSC).

### P2 Embodiment 21

The composition of P2 embodiment 20, wherein said chimeric poxvirus forms part of said NSC.

### P2 Embodiment 22

The composition of P2 embodiment 20 or 21, wherein said poxvirus is encoded by a nucleic acid sequence having a sequence identity of at least 70% to SEQ ID NO:1.

### P2 Embodiment 23

The composition of P2 embodiment 22, wherein said nucleic acid sequence comprises nucleic acid fragments from at least two poxvirus strains selected from the group consisting of cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, vaccinia virus strain AS, orf virus strain NZ2 and pseudocowpox virus strain TJS.

### P2 Embodiment 24

The composition of P2 embodiment 23 or 23, wherein said nucleic acid sequence comprises nucleic acid fragments from cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic and vaccinia virus strain AS.

### P2 Embodiment 25

The composition of one of P2 embodiment 22-24, wherein said nucleic acid sequence further comprises one or more anti-cancer nucleic acid sequences or a detectable moiety-encoding nucleic acid sequence.

### P2 Embodiment 26

The composition of one of P2 embodiments 20-25, wherein said chimeric poxvirus is a replication-competent chimeric poxvirus.

### P2 Embodiment 27

The composition of one of P2 embodiments 20-26, wherein said

NSC is a Human Leukocyte Antigen (HLA) II-negative NSC.

### P2 Embodiment 28

The composition of one of P2 embodiments 20-26, wherein said NSC is an HB1.F3.CD21 cell.

### P2 Embodiment 29

The composition of one of P2 embodiment 20-28, further comprising an effective amount of a therapeutic agent.

### P2 Embodiment 30

The composition of P2 embodiment 29, wherein said therapeutic agent is an anti-cancer agent.

### P2 Embodiment 31

The composition of P2 embodiment 29 or 30, wherein said therapeutic agent is a PD-L1 inhibitor, a CTLA-4 inhibitor or an OX40 inhibitor.

### P2 Embodiment 32

The composition of one of P2 embodiments 20-31, wherein said composition is effective to treat cancer.

### P2 Embodiment 33

The composition of P2 embodiment 32, wherein said cancer is breast cancer, colon cancer, kidney cancer, leukemia, lung cancer, melanoma, ovarian cancer, prostate cancer, pancreatic cancer, brain cancer, liver cancer, gastric cancer or a sarcoma.

### P2 Embodiment 34

The composition of P2 embodiment 33, wherein said ovarian cancer is an ovarian metastasis.

### P2 Embodiment 35

The composition of one of P2 embodiment 20-34, wherein said composition is a pharmaceutical composition.

### P2 Embodiment 36

The composition of P2 embodiment 35, wherein said pharmaceutical composition comprises a pharmaceutically acceptable excipient.

### P2 Embodiment 37

A neural stem cell (NSC) comprising a chimeric poxvirus.

### P2 Embodiment 38

The neural stem cell of P2 embodiment 37, wherein said poxvirus is encoded by a nucleic acid sequence having a sequence identity of at least 70% to SEQ ID NO:1.

### P2 Embodiment 39

The neural stem cell of P2 embodiment 38, wherein said nucleic acid sequence comprises nucleic acid fragments from at least two poxvirus strains selected from the group consisting of cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, vaccinia virus strain AS, orf virus strain NZ2 and pseudocowpox virus strain TJS.

### P2 Embodiment 40

The neural stem cell of P2 embodiment 38 or 39, wherein said nucleic acid sequence comprises nucleic acid fragments from cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic and vaccinia virus strain AS.

### P2 Embodiment 41

The neural stem cell of one of P2 embodiments 38-40, wherein said nucleic acid sequence further comprises one or more anti-cancer nucleic acid sequences or a detectable moiety-encoding nucleic acid sequence.

### P2 Embodiment 42

The neural stem cell of one of P2 embodiments 37-41, wherein said chimeric poxvirus is a replication-competent chimeric poxvirus.

### P2 Embodiment 43

The neural stem cell of one of P2 embodiments 37-42, wherein said NSC is a Human Leukocyte Antigen (HLA) II-negative NSC.

### P2 Embodiment 44

The neural stem cell of one of P2 embodiments 37-43, wherein said NSC is an HB1.F3.CD21 cell

### P2 Embodiment 45

The neural stem cell of one of P2 embodiment 37-38, wherein said NSC forms part of a pharmaceutical composition.

### P2 Embodiment 46

The neural stem cell of P2 embodiment 44, wherein said pharmaceutical composition comprises a pharmaceutically acceptable excipient.

### P2 Embodiment 47

A method of treating cancer, said method comprising administering to a subject in need thereof an effective amount of a neural stem cell of one of embodiments 37-46.

### P2 Embodiment 48

The method of P2 embodiment 47, wherein said cancer is breast cancer, colon cancer, kidney cancer, leukemia, lung cancer, melanoma, ovarian cancer, prostate cancer, pancreatic cancer, brain cancer, liver cancer, gastric cancer or a sarcoma

### P2 Embodiment 49

The method of P2 embodiment 47 or 48, wherein said ovarian cancer is an ovarian metastasis.

What is claimed is:

1. A method of treating cancer, said method comprising administering to a subject in need thereof an effective amount of a chimeric poxvirus and a neural stem cell (NSC).

2. The method of claim 1, wherein said chimeric poxvirus forms part of said NSC.

3. The method of claim 2, wherein said NSC is administered intraperitonally.

4. The method of claim 2, wherein said NSC is administered at a cell number of 1×10⁸ to 2×10⁸ cells.

5. The method of claim 2, wherein said NSC is administered at a cell number of 1.5×10⁸ cells.

6. The method of claim 2, wherein said NSC is administered at a cell number of 5×10⁶ to 5×10⁷ cells/ml.

7. The method of claim 1, wherein said chimeric poxvirus is encoded by a nucleic acid sequence having a sequence identity of at least 70% to SEQ ID NO:1.

8. The method of claim 7, wherein said nucleic acid sequence comprises nucleic acid fragments from at least two poxvirus strains selected from the group consisting of cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, vaccinia virus strain AS, orf virus strain NZ2 and pseudocowpox virus strain TJS.

9. The method of claim 8, wherein said nucleic acid sequence comprises nucleic acid fragments from cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic and vaccinia virus strain AS.

10. The method of claim 7, wherein said nucleic acid sequence further comprises one or more anti-cancer nucleic acid sequences or a detectable moiety-encoding nucleic acid sequence.

11. The method of claim 1, wherein said chimeric poxvirus is a replication-competent chimeric poxvirus.

12. The method of claim 1, wherein said NSC is a Human Leukocyte Antigen (HLA) II-negative NSC.

13. The method of claim 1, wherein said NSC is an HB1.F3.CD21 cell.

14. The method of claim 1, the method comprising further administering an effective amount of a therapeutic agent.

15. The method of claim 14, wherein said therapeutic agent is an anti-cancer agent.

16. The method of claim 14, wherein said therapeutic agent is selected from the group consisting of a small molecule, a nucleic acid, a polypeptide and an antibody.

17. The method of claim 14, wherein said therapeutic agent is a PD-L1 inhibitor, a CTLA-4 inhibitor or an OX40 inhibitor.

18. The method of claim 1, wherein said cancer is breast cancer, colon cancer, kidney cancer, leukemia, lung cancer, melanoma, ovarian cancer, prostate cancer, pancreatic cancer, brain cancer, liver cancer, gastric cancer or a sarcoma.

19. The method of claim 18, wherein said ovarian cancer is an ovarian metastasis.

20. A composition comprising a chimeric poxvirus and a neural stem cell (NSC).

21. The composition of claim 20, wherein said chimeric poxvirus forms part of said NSC.

22. The composition of claim 20, wherein said poxvirus is encoded by a nucleic acid sequence having a sequence identity of at least 70% to SEQ ID NO:1.

23. The composition of claim 22, wherein said nucleic acid sequence comprises nucleic acid fragments from at least two poxvirus strains selected from the group consisting of cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, vaccinia virus strain AS, orf virus strain NZ2 and pseudocowpox virus strain TJS.

24. The composition of claim 22, wherein said nucleic acid sequence comprises nucleic acid fragments from cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic and vaccinia virus strain AS

25. The composition of claim 22, wherein said nucleic acid sequence further comprises one or more anti-cancer nucleic acid sequences or a detectable moiety-encoding nucleic acid sequence.

26. The composition of claim 20, wherein said chimeric poxvirus is a replication-competent chimeric poxvirus.

27. The composition of claim 20, wherein said NSC is a Human Leukocyte Antigen (HLA) II-negative NSC.

28. The composition of claim 20, wherein said NSC is an HB1.F3.CD21 cell.

29. The composition of claim 20, further comprising an effective amount of a therapeutic agent.

30. The composition of claim 29, wherein said therapeutic agent is an anti-cancer agent.

31. The composition of claim 29, wherein said therapeutic agent is a PD-L1 inhibitor, a CTLA-4 inhibitor or an OX40 inhibitor.

32. The composition of claim 20, wherein said composition is effective to treat cancer.

33. The composition of claim 32, wherein said cancer is breast cancer, colon cancer, kidney cancer, leukemia, lung cancer, melanoma, ovarian cancer, prostate cancer, pancreatic cancer, brain cancer, liver cancer, gastric cancer or a sarcoma.

34. The composition of claim 33, wherein said ovarian cancer is an ovarian metastasis.

35. The composition of claim 20, wherein said composition is a pharmaceutical composition.

36. The composition of claim 35, wherein said pharmaceutical composition comprises a pharmaceutically acceptable excipient.

37. A neural stem cell (NSC) comprising a chimeric poxvirus.

38. The neural stem cell of claim 37, wherein said poxvirus is encoded by a nucleic acid sequence having a sequence identity of at least 70% to SEQ ID NO:1.

39. The neural stem cell of claim 38, wherein said nucleic acid sequence comprises nucleic acid fragments from at least two poxvirus strains selected from the group consisting of cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, vaccinia virus strain AS, orf virus strain NZ2 and pseudocowpox virus strain TJS.

40. The neural stem cell of claim 38, wherein said nucleic acid sequence comprises nucleic acid fragments from cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic and vaccinia virus strain AS.

41. The neural stem cell of claim 38, wherein said nucleic acid sequence further comprises one or more anti-cancer nucleic acid sequences or a detectable moiety-encoding nucleic acid sequence.

42. The neural stem cell of claim 37, wherein said chimeric poxvirus is a replication-competent chimeric poxvirus.

43. The neural stem cell of claim 37, wherein said NSC is a Human Leukocyte Antigen (HLA) II-negative NSC.

44. The neural stem cell of claim 37, wherein said NSC is an HB1.F3.CD21 cell.

45. The neural stem cell of claim 37, wherein said NSC forms part of a pharmaceutical composition.

46. The neural stem cell of claim 45, wherein said pharmaceutical composition comprises a pharmaceutically acceptable excipient.

47. A method of treating cancer, said method comprising administering to a subject in need thereof an effective amount of a neural stem cell of claim 37.

48. The method of claim 47, wherein said cancer is breast cancer, colon cancer, kidney cancer, leukemia, lung cancer, melanoma, ovarian cancer, prostate cancer, pancreatic cancer, brain cancer, liver cancer, gastric cancer or a sarcoma.

49. The method of claim 47, wherein said ovarian cancer is an ovarian metastasis.
